(19) United States
(12) Patent Application Publication
(10) Pub. No.: US 2019/0015400 A1
(43) Pub. Date: Jan. 17, 2019

(54) Antifungal Compositions

(71) Applicant: University of Kentucky Research Foundation, Lexington, KY (US)

(72) Inventors: Sylvie Garneau-Tsodikova, Lexington, KY (US); Atefeh Garzan, Lexington, KY (US); Selina Yijia Li Holbrook, Lexington, KY (US); Emily Kristen Dennis, Lexington, KY (US)

(21) Appl. No.: 16/038,171

(22) Filed: Jul. 17, 2018

Related U.S. Application Data
(60) Provisional application No. 62/533,303, filed on Jul. 17, 2017.

(51) Int. Cl. A61K 31/451; A61K 31/45; A61P 31/10; A01N 43/40; A61K 31/7048

(52) U.S. Cl. CPC A61K 31/451; A61K 31/45; A61P 31/10; A61K 31/4196; A61K 31/7048; A61K 2300/00; A01N 43/40

(57) ABSTRACT

Provided herein are antifungal compositions and methods of use thereof. The antifungal compositions include an antifungal agent and an antipsychotic agent or an antihistamine. The methods of use thereof include administering a composition including an antifungal agent and an antipsychotic or an antihistamine to a plant or animal in need thereof.

### RELATED APPLICATIONS

This application claims the benefit of U.S. Provisional Application Ser. No. 62/533,303, filed Jul. 17, 2017, the entire disclosure of which is incorporated herein by this reference.

### TECHNICAL FIELD

The presently-disclosed subject matter generally relates to antifungal compositions. In particular, certain embodiments of the presently-disclosed subject matter relate to compositions having improved antifungal activity and reduced adverse side effects.

### BACKGROUND

Fungal infections are an ever-growing global health problem. Even though fungal infections can affect both the healthy and the sick, they pose a much greater threat to chronically ill and immunocompromised patients. As the number of hospitalized and immunocompromised patients increases, the rise of fungal infections and the associated resistance problems are raising alarm. Candida albicans is a leading cause of fungal infections and accounts for up to 70% of total incidents worldwide. Invasive candidiasis, a bloodstream infection of Candida species, is one of the most common nosocomial fungal diseases with an estimate of 350,000 incidents worldwide every year and a 30-55% mortality rate. From 2000 to 2005, the number of invasive candidiasis in the USA rose by 52% and inevitably much more in less developed countries, such as Brazil and India. In addition to Candida species, some filamentous fungi, such as Aspergillus, also cause life-threatening infections. If left untreated, invasive aspergillosis can result in a 99% mortality rate, and therefore, is another fungal infection that is calling for immediate attention worldwide. For instance, the increasing use of corticosteroid in 4.8 million asthma patients is linked to over 400,000 patients developing chronic pulmonary aspergillosis.

Fungi infect not only humans but also various food sources. From the Irish potato famine in the 19^{th} century to today's spread of Puccina graminis tritici Ug99, which is responsible for stem or black rust diseases on wheat, fungal infections have never been solely a burden to clinical healthcare providers, but also to food production and quality control professionals. By infecting crops and livestock, fungi not only result in severe damage in the food production industry, but can be spread to humans through food and cause diseases.

The challenges faced with fungal infections are evident and urgent, yet, the repertoire of antifungals is limited. Not only is the repertoire limited, the antifungal drugs which are available for the treatment of systemic infections all possess severe limitations as well. For example, the intravenous drug amphotericin B (AmB) was the first antifungal agent approved for systemic use for invasive fungal infections over 50 years ago. However, its dose-limiting toxicity and other adverse effects often result in interruption of treatment courses. Another intravenous antifungal drug family, the echinocandins (e.g., caspofungin (CAS)), was shown to possess lower toxicity and to be better tolerated than AmB in various formulations. The broad-spectrum nature of the echinocandins has made them a better alternative to AmB for treating invasive fungal infections. Nonetheless, echinocandins still possess sever limitations.

The azoles (e.g., fluconazole (FLC), itraconazole (ITC), ketoconazole (KTC), posaconazole (POS), and voriconazole (VOR)) represent another family of antifungals, and are the only class of oral antifungals identified due to their excellent oral bioavailability. By targeting the sterol 14α-demethylase, azoles inhibit the biosynthesis of ergosterol, which is a vital component of the fungal cell membrane. Inhibition of this enzyme causes the methylated sterol side products to accumulate inside the fungal cells, which is toxic to fungi and results in cell death. In addition to excellent bioavailability, the azole antifungals also exhibit fewer adverse effects than AmB. Consequently, the azole antifungals quickly became the most clinically prescribed antifungal class worldwide since their introduction to the market.

However, azoles have their own limitations. Azoles inhibit cytochrome P450 enzymes, which results in undesired interference with the metabolism of numerous other drugs, making it difficult for patients being treated with multiple concurrent medications. Moreover, azoles are often found to be fungistatic towards many fungi, resulting in only temporary inhibition of fungal growth. The lack of fungicidal activity has made it challenging to prevent fungal regrowth and the accompanied development of antimicrobial resistance to azoles. The development of resistance against the azole antifungals are gradually causing these once-effective medications to lose their potency, as more and more fungal clinical isolates are being reported to be resistance to azoles. Furthermore, azoles impose severe toxicity on multiple organs and systems in the human body, such as kidney and digestive system.

Accordingly, there is a need for antifungals and antifungal treatments which overcome the drawbacks associated with existing compounds.

### SUMMARY

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of features.

The presently-disclosed subject matter provides, in some embodiments, a compound selected from the group:

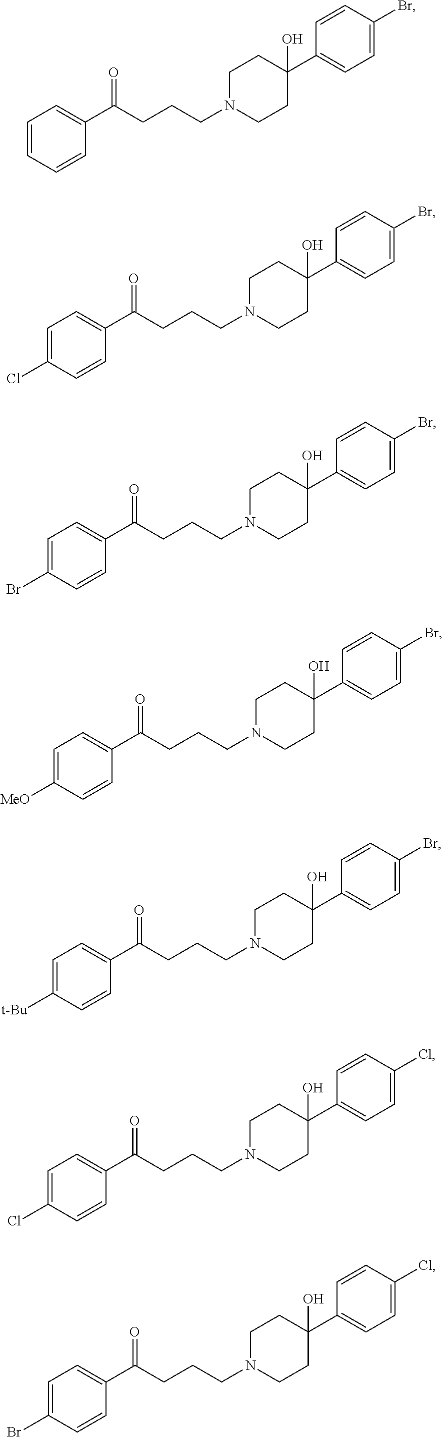

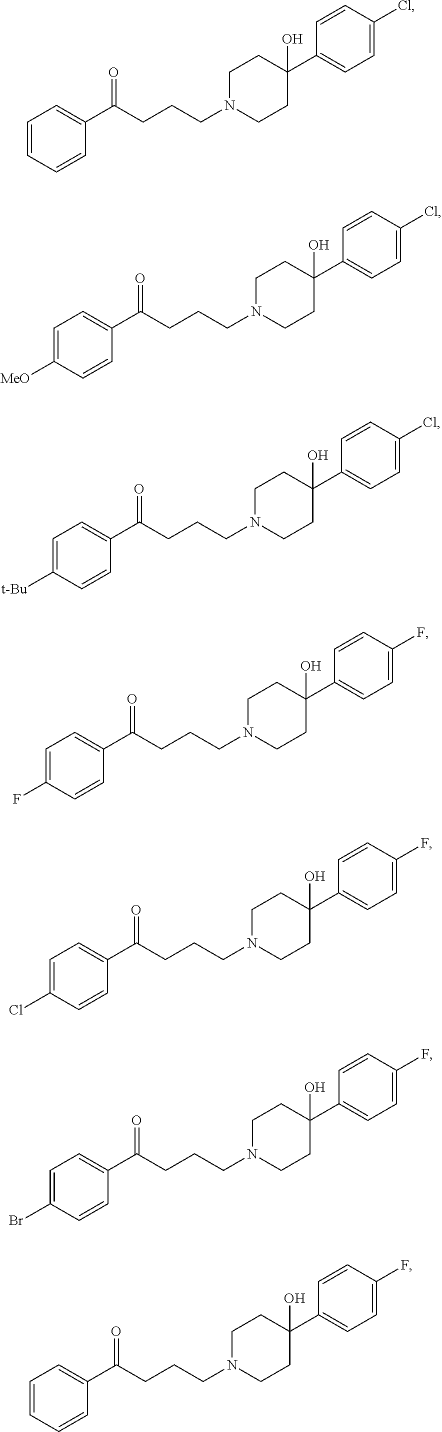

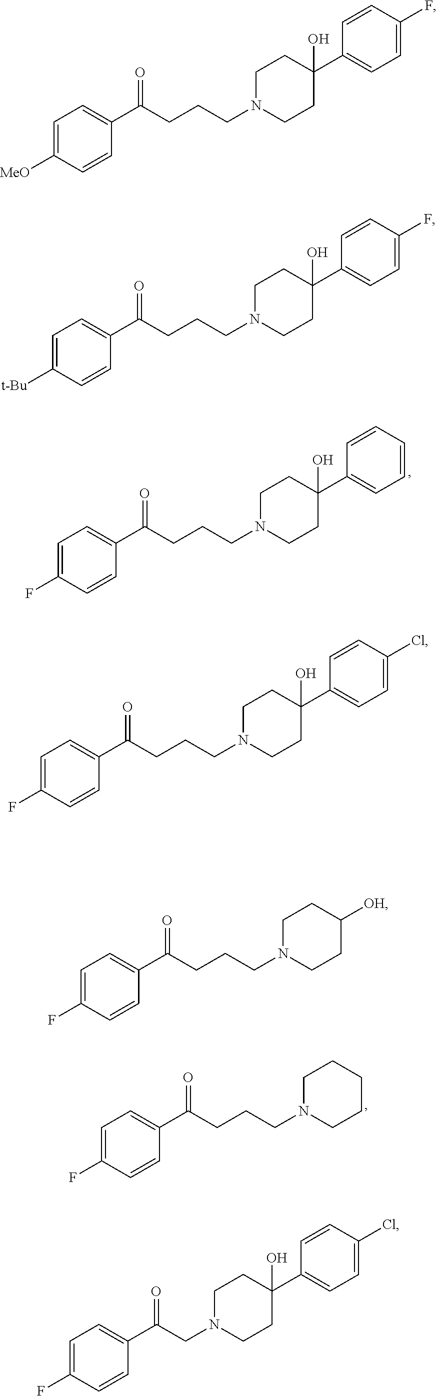

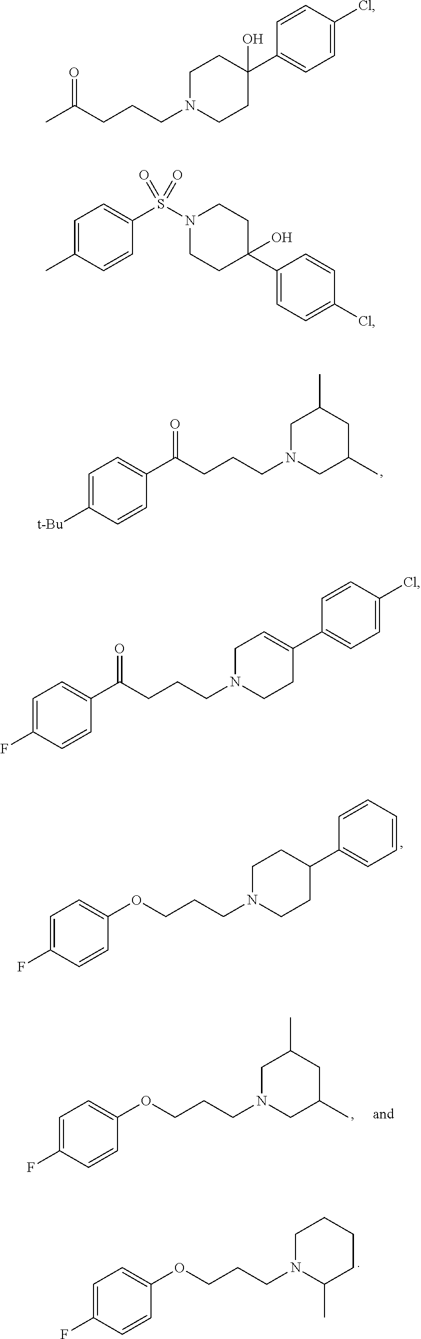

Also provided herein, in some embodiments is a composition including an antifungal agent and a compound selected from

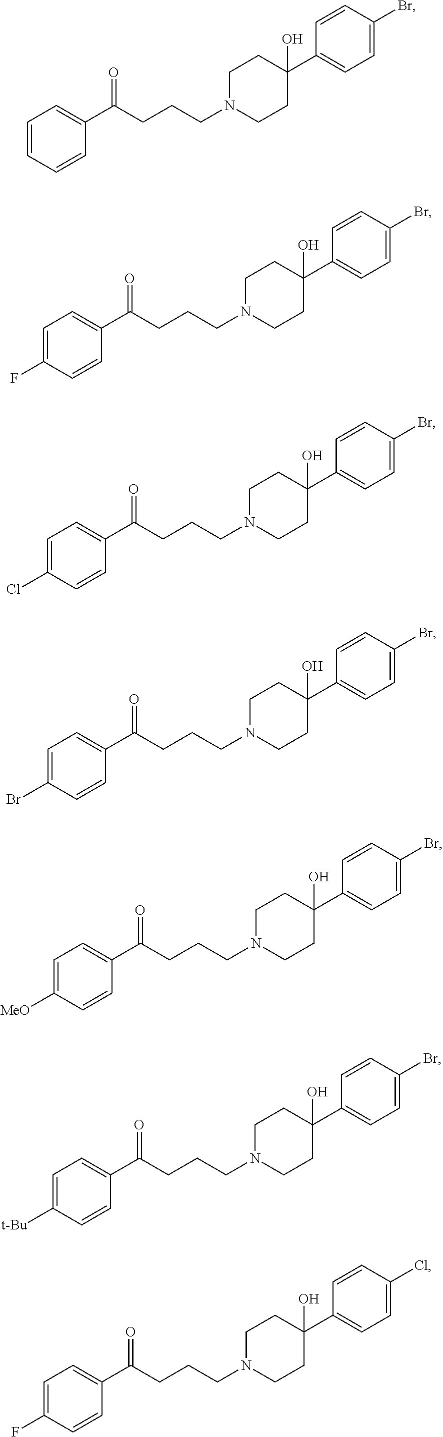

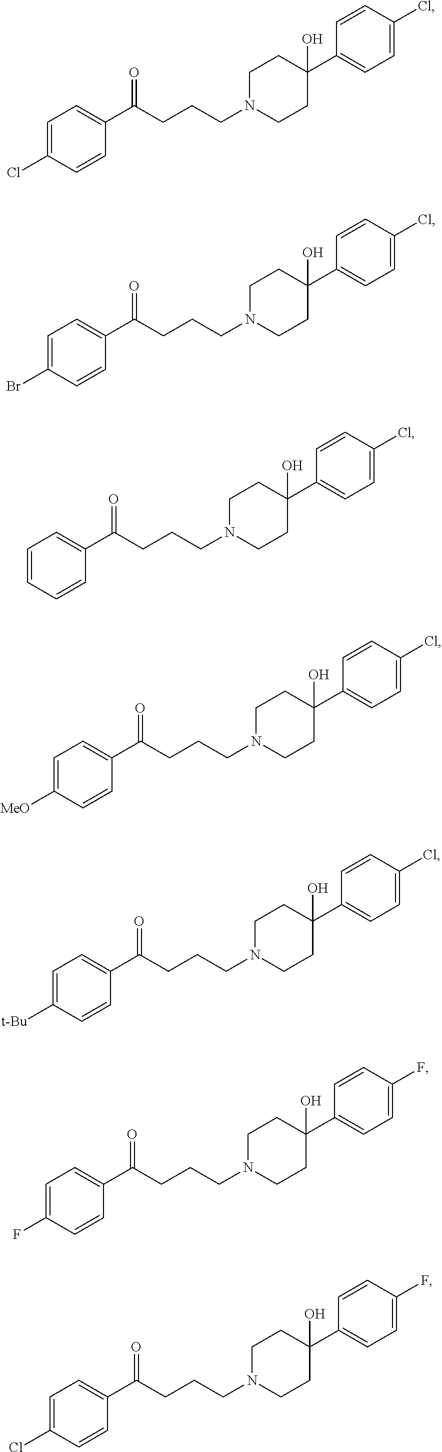

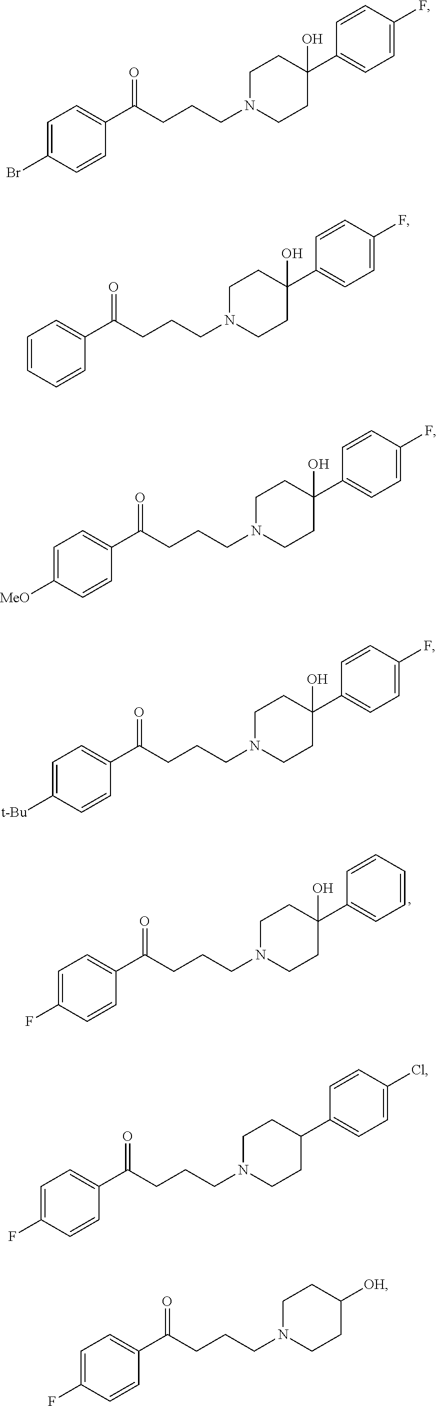

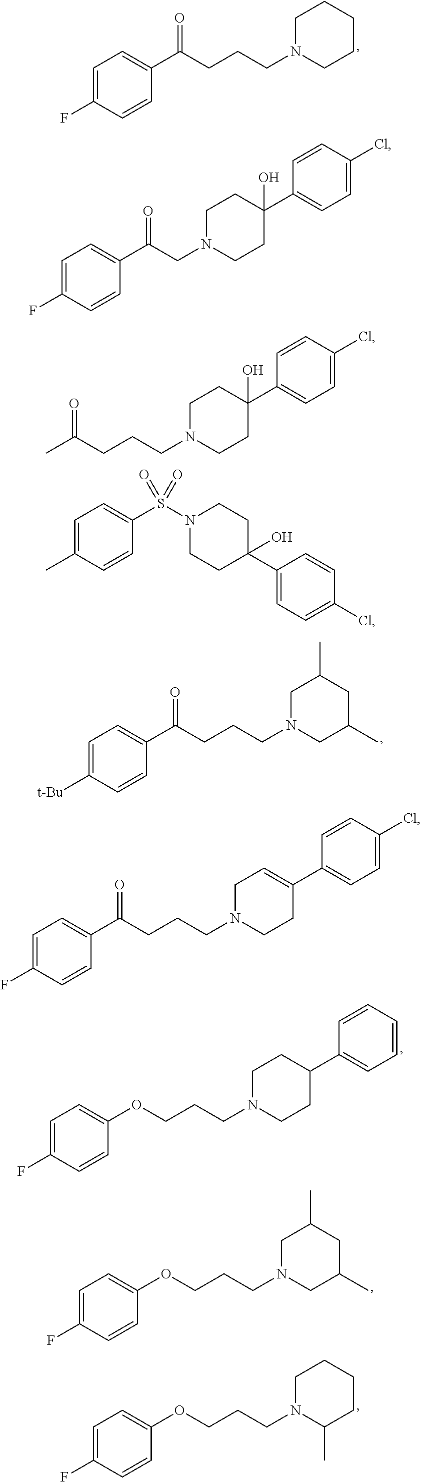

terfenadine (TERF), ebastine (EBA), and derivatives thereof.

In some embodiments, the antifungal agent is an azole antifungal agent. In some embodiments, the antifungal agent is a non-azole antifungal agent. In one embodiment, the antifungal agent is selected from the group consisting of: fluconazole (FLC), itraconazole (ITC), ketoconazole (KTC), posaconazole (POS), and voriconazole (VOR), ketoconazolev(KTC), undecylic acid (undecanoic acid), nystatin (NYS), naftifine (NAF), tolnaftate, amorolfine, butenafine (BTF), miconazole (MCZ), econazole, ciclopirox, oxiconazole, sertaconazole, efinaconazole, clotrimazole (CLO), sulconazole, tioconazole, tavaborole, terbinafine (TER), mancozeb, tricyclazole, carbendazim, hexaconazole, propineb, metalaxyl, benomyl (BEN) (Methyl 1-(butylcarbamoyl)-2-benzimidazolecarbamate), difenoconazole, propiconazole (PCZ), kitazin, tebuconazole (TER), tridemorph (TDM), and metconazole (MET).

In some embodiments, the composition has increased antifungal activity as compared to the antifungal agent alone. In some embodiments, composition has synergistically increased antifungal activity as compared to the antifungal agent alone. In some embodiments, the ratio of antifungal agent to compound is between about 1:1 and 1:1100, between about 1:1 and 1:5, between about 1:1 and 1:10, between about 1:1 and 1:100, between about 1:1 and 1:300, between about 1:1 and 1:500, or between about 1:10 and 1:500. In some embodiments, the effective amount of the antifungal agent in the composition is less than the effective amount of the antifungal agent when used alone.

Further provided herein, in some embodiments, is a method of treating a fungal infection comprising administering a composition including an antifungal agent and an antihistamine or antipsychotic agent to an infected plant. In one embodiment, the infection is caused by a plant pathogen.

Still further provided herein, in some embodiments, is a method of treating a fungal infection comprising administering a composition including an antifungal agent and an antihistamine or antipsychotic agent to a subject in need thereof. In one embodiment, the infection is caused by an animal pathogen. In one embodiment, the infection is caused by a human pathogen.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-B show structures of (A) the common azole antifungals with the triazole pharmacophore highlighted in pink as well as (B) the chemical synthesis of bromperidol series compounds (1-5) involved in this study.

FIGS. 2A-B show time- and dose-dependent antifungal synergy of the combination of (A) POS or (B) VOR and compound 2 at various concentrations against C. albicans ATCC 64124 (strain F). Each sample with resazurin added for visualization of fungal growth is presented underneath the growth curve for each combination.

FIGS. 3A-D show mammalian cytotoxicity evaluation of (A) POS and VOR alone; (B) compound 2 alone; and representative combinations of azoles (POS or VOR) at various concentrations along with compound 2 at (C) 8 μg/mL or (D) 32 μg/mL supplemented in the media against BEAS-2B, HEK-293, and J774A.1 cells. Note: As at 32 μg/mL, compound 2 exert toxicity against J774A.1 (as seen in panel B), this cell line was not tested in panel D.

FIG. 4 shows ¹H NMR spectrum for compound 1 in CDCl₃.

FIG. 5 shows ¹³C NMR spectrum for compound 1 in CDCl₃.

FIG. 6 shows HPLC trace for compound 1. Rt=7.75 min.

FIG. 7 shows ¹H NMR spectrum for compound 2 in CDCl₃.

FIG. 8 shows ¹³C NMR spectrum for compound 2 in CDCl₃.

FIG. 9 shows HPLC trace for compound 2. Rt=7.93 min.

FIG. 10 shows ¹H NMR spectrum for compound 3 in CDCl₃.

FIG. 11 shows ¹³C NMR spectrum for compound 3 in CDCl₃.

FIG. 12 shows HPLC trace for compound 3. Rt=8.54 min.

FIG. 13 shows ¹H NMR spectrum for compound 4 in CDCl₃.

FIG. 14 shows ¹³C NMR spectrum for compound 4 in (CD₃)₂SO.

FIG. 15 shows HPLC trace for compound 4. Rt=8.66 min.

FIG. 16 shows ¹H NMR spectrum for compound 5 in CDCl₃.

FIG. 17 shows ¹³C NMR spectrum for compound 5 in CD₃OD.

FIG. 18 shows HPLC trace for compound 5. Rt=8.22 min.

FIG. 19 shows ¹H NMR spectrum for compound 6 in CDCl₃.

FIG. 20 shows ¹³C NMR spectrum for compound 6 in CDCl₃.

FIG. 21 shows HPLC trace for compound 6. Rt=9.84 min.

FIG. 22 shows ¹H NMR spectrum for compound 7 in CDCl₃.

FIG. 23 shows ¹³C NMR spectrum for compound 7 in CDCl₃.

FIG. 24 shows HPLC trace for compound 7. Rt=7.62 min.

FIG. 25 shows ¹H NMR spectrum for compound 8 in CDCl₃.

FIG. 26 shows ¹³C NMR spectrum for compound 8 in CDCl₃.

FIG. 27 shows HPLC trace for compound 8. Rt=8.34 min.

FIG. 28 shows ¹H NMR spectrum for compound 9 in CDCl₃.

FIG. 29 shows ¹³C NMR spectrum for compound 9 in (CD₃)₂SO.

FIG. 30 shows HPLC trace for compound 9. Rt=8.63 min.

FIG. 31 shows ¹H NMR spectrum for compound 10 in CD₃OD.

FIG. 32 shows ¹³C NMR spectrum for compound 10 in CD₃OD.

FIG. 33 shows HPLC trace for compound 10. Rt=7.60 min.

FIG. 34 shows ¹H NMR spectrum for compound 11 in CDCl₃.

FIG. 35 shows ¹³C NMR spectrum for compound 11 in CDCl₃.

FIG. 36 shows HPLC trace for compound 11. Rt=7.94 min.

FIG. 37 shows ¹H NMR spectrum for compound 12 in CDCl₃.

FIG. 38 shows ¹³C NMR spectrum for compound 12 in CDCl₃.

FIG. 39 shows HPLC trace for compound 12. Rt=9.48 min.

FIG. 40 shows ¹H NMR spectrum for compound 13 in CDCl₃.

FIG. 41 shows ¹³C NMR spectrum for compound 13 in CDCl₃.

FIG. 42 shows HPLC trace for compound 13. Rt=7.06 min.

FIG. 43 shows ¹H NMR spectrum for compound 14 in CDCl₃.

FIG. 44 shows ¹³C NMR spectrum for compound 14 in CDCl₃.

FIG. 45 shows HPLC trace for compound 14. Rt=7.82 min.

FIG. 46 shows ¹H NMR spectrum for compound 15 in CDCl₃.

FIG. 47 shows ¹³C NMR spectrum for compound 15 in CDCl₃.

FIG. 48 shows HPLC trace for compound 15. Rt=7.96 min.

FIG. 49 shows ¹H NMR spectrum for compound 16 in CDCl₃.

FIG. 50 shows ¹³C NMR spectrum for compound 16 in CDCl₃.

FIG. 51 shows HPLC trace for compound 16. Rt=6.88 min.

FIG. 52 shows ¹H NMR spectrum for compound 17 in CDCl₃.

FIG. 53 shows ¹³C NMR spectrum for compound 17 in CDCl₃.

FIG. 54 shows HPLC trace for compound 17. Rt=7.44 min.

FIG. 55 shows ¹H NMR spectrum for compound 18 in CD₃OD.

FIG. 56 shows ¹³C NMR spectrum for compound 18 in CD₃OD.

FIG. 57 shows HPLC trace for compound 18. Rt=9.12 min.

FIG. 58 shows ¹H NMR spectrum for compound 19 in CD₃OD.

FIG. 59 shows HPLC trace for compound 19. Rt=6.44 min.

FIG. 60 shows ¹H NMR spectrum for compound 20 in CDCl₃.

FIG. 61 shows HPLC trace for compound 20. Rt=7.71 min.

FIG. 62 shows ¹H NMR spectrum for compound 21 in CDCl₃.

FIG. 63 shows HPLC trace for compound 21. Rt=4.69 min.

FIG. 64 shows ¹H NMR spectrum for compound 22 in CDCl₃.

FIG. 65 shows HPLC trace for compound 22. Rt=6.99 min.

FIG. 66 shows ¹H NMR spectrum for compound 23 in CDCl₃.

FIG. 67 shows ¹³C NMR spectrum for compound 23 in CDCl₃.

FIG. 68 shows HPLC trace for compound 23. Rt=6.85 min.

FIG. 69 shows ¹H NMR spectrum for compound 24 in CDCl₃.

FIG. 70 shows ¹³C NMR spectrum for compound 24 in CDCl₃.

FIG. 71 shows HPLC trace for compound 24. Rt=6.28 min.

FIG. 72 shows ¹H NMR spectrum for compound 25 in CD₃OD.

FIG. 73 shows ¹³C NMR spectrum for compound 25 in CD₃OD.

FIG. 74 shows HPLC trace for compound 25. Rt=9.89 min.

FIG. 75 shows ¹H NMR spectrum for compound 26 in CDCl₃.

FIG. 76 shows ¹³C NMR spectrum for compound 26 in CDCl₃.

FIG. 77 shows HPLC trace for compound 26. Rt=8.84 min.

FIG. 78 shows ¹H NMR spectrum for compound 27 in CDCl₃.

FIG. 79 shows HPLC trace for compound 27. Rt=8.57 min.

FIG. 80 shows ¹H NMR spectrum for compound 28 in CDCl₃.

FIG. 81 shows ¹³C NMR spectrum for compound 28 in CDCl₃.

FIG. 82 shows HPLC trace for compound 28. Rt=7.71 min.

FIG. 83 shows ¹H NMR spectrum for compound 29 in CDCl₃.

FIG. 84 shows ¹³C NMR spectrum for compound 29 in CDCl₃.

FIG. 85 shows HPLC trace for compound 29. Rt=7.29 min.

FIG. 86 shows ¹H NMR spectrum for compound 30 in CDCl₃.

FIG. 87 shows ¹³C NMR spectrum for compound 30 in CDCl₃.

FIG. 88 shows HPLC trace for compound 30. Rt=7.61 min.

FIGS. 89A-D show structures of (A) azole antifungals clotrimazole (CLO), fluconazole (FLC), ketoconazole (KTC), itraconazole (ITC), miconazole (MCZ), posaconazole (POS), and voriconazole (VRC); (B) haloperidol, an antipsychotic; and antihistamine agents (C) terfenadine (TERF) and (D) ebastine (EBA).

FIGS. 90A-B show representative time-kill curves for POS and EBA against (A) C. albicans ATCC 10231 (strain B) and (B) C. glabrata ATCC 2001 (strain H). Fungal strains were treated with no drug (black circles), EBA (white circle), POS (inverted black triangle), 1×MIC (white triangle), 4×MIC (black square), AMB at 1×MIC (grey square), and NYT at 1×MIC (white square). We further verified the number of CFU/mL at the 24-hour end point by adding resazurin to the cultures. Actively replicating fungal cells metabolize resazurin, which is a blue-purple color, to produce resorufin, which has a pink-orange color. In cultures where there is little to no active cells, the culture remains a blue-purple. Where there is a low number of CFU, the culture appears a red color, and where there are many cells, the culture appears pink to orange. Treated cultures with resazurin added for visualization of fungal growth are shown below the growth curves for each strain (a=sterile control; b=no drug; c=POS; d=EBA; e=EBA and POS combination at 1×MIC; f=EBA and POS combination at 4×MIC, g=AMB at 1×MIC, and NYT at 1×MIC).

FIGS. 91A-H show 96-well plates showing the anti-biofilm activity of the POS and EBA combinations against (A-B) C. albicans ATCC 10231 (strain B), (C-D) C. albicans ATCC 64124 (strain F), and (E-F) C. glabrata ATCC 2001 (strain H). For comparison to fungicidal control, SMIC of the polyenes AMB and NYT are shown against (G) C. albicans ATCC 10231 (strain B) and (H) C. glabrata ATCC 2001 (strain H). SC indicates sterile controls where no fungal cells were added to the wells.

FIGS. 92A-C show representative cytotoxicity assays against 3 mammalian cell lines of (A) POS alone, (B) EBA alone, and (C) POS+3.1 μg/mL EBA. Cells lines included A549 (pink bars), BEAS-2B (blue bars), and HEK-293 (orange bars). The positive control consisted of cells treated with Triton X-100® (TX, 12.5% v/v). The negative control consisted of cells treated with DMSO (no drug).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### Definitions

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the presently-disclosed subject matter, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “effective amount” refers to an amount that is sufficient to achieve the desired result, i.e., antifungal activity.

As used herein, the term “subject” refers to a target of administration.

As used herein, the terms “treatment” or “treating” relate to any treatment of a fungal infection, including but not limited to prophylactic treatment (pre-infection), and therapeutic treatment (post-infection). As such, the terms treatment or treating include, but are not limited to: preventing an infection or the development of an infection; inhibiting the progression of an infection; arresting or preventing the development of an infection; reducing the severity of an infection; ameliorating or relieving symptoms associated with an infection; and causing a regression of an infection or one or more of the symptoms associated with an infection.

As used herein, “synergy,” “synergistically,” “synergism,” and “synergistic effect” can refer to any substantial enhancement, in a composition of at least two compounds, of a measurable effect. In particular, synergism is a well-understood feature in the art, where components of a composition potentiate each other's effect. Synergy is a specific feature of a combination of components, and is above any background level of enhancement that would be due solely to, e.g., additive effects of any random combination of ingredients. The fractional inhibitory concentration index (FICI) is the most well accepted standard in determining synergy in drug combination studies. Synergism is defined as an FICI=0.5. Additive effect, where the total effect is an addition of the individual effect from each component, is defined as 0.5<FICI=4. Antagonism, where two drugs inhibit each other's action, is defined as FICI>4.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Where reference is made to a URL or other such identifier or address, it understood that such identifiers can change and particular information on the internet can come and go, but equivalent information can be found by searching the internet. Reference thereto evidences the availability and public dissemination of such information.

All combinations of method or process steps as used herein can be performed in any order, unless otherwise specified or clearly implied to the contrary by the context in which the referenced combination is made.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds, compositions, and methods useful for treating fungal infections. In some embodiments, the presently-disclosed subject matter includes compositions comprising an antifungal agent and an antipsychotic agent. In one embodiment, for example, the compositions include an antifungal agent, and an antipsychotic agent having a structure according to Formula I:

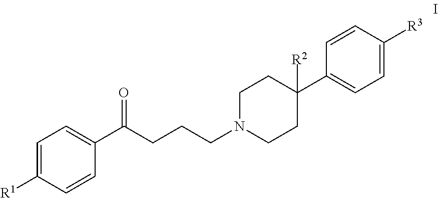

Where R¹ includes, but is not limited to, H, F, Cl, Br, MeO, or t-Bu; R² includes, but is not limited to, H or OH; and R³ includes, but is not limited to, Br, Cl, F, or H. Suitable antipsychotic derivatives according to Formula I include, but are not limited to:

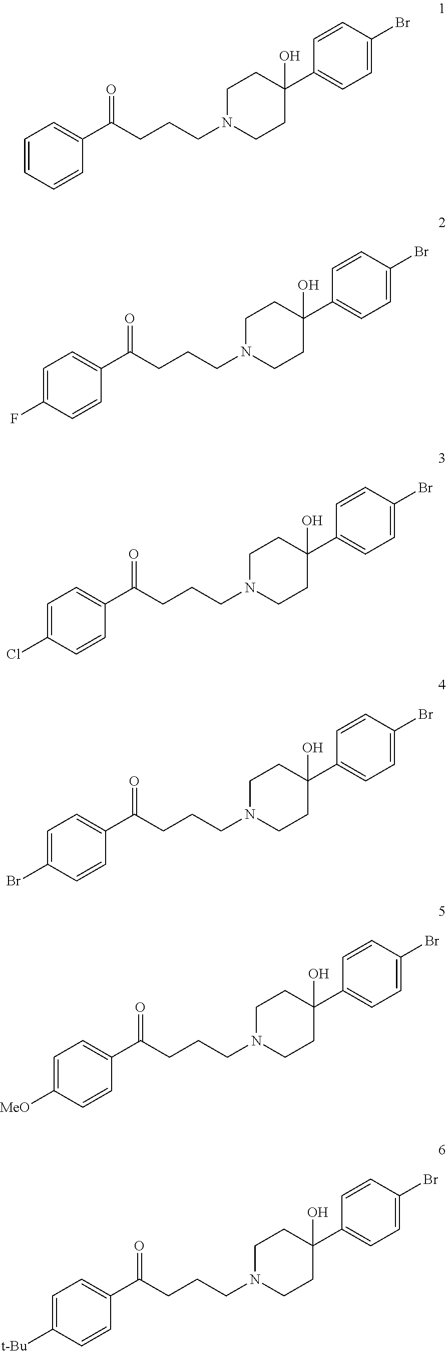 [SMILES: O=C(CCCN1CCC(O)(c2ccc(Br)cc2)CC1)c1ccccc1 ; O=C(CCCN1CCC(O)(c2ccc(Br)cc2)CC1)c1ccc(F)cc1 ; O=C(CCCN1CCC(O)(c2ccc(Br)cc2)CC1)c1ccc(Cl)cc1 ; O=C(CCCN1CCC(O)(c2ccc(Br)cc2)CC1)c1ccc(Br)cc1 ; COc1ccc(C(=O)CCCN2CCC(O)(c3ccc(Br)cc3)CC2)cc1 ; CC(C)(C)c1ccc(C(=O)CCCN2CCC(O)(c3ccc(Br)cc3)CC2)cc1]

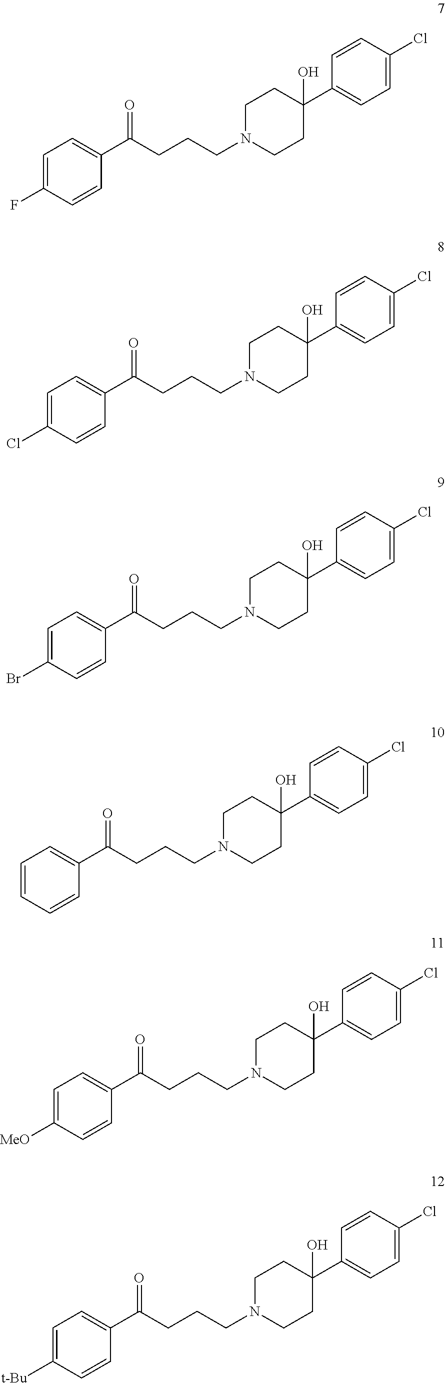 [SMILES: O=C(CCCN1CCC(O)(c2ccc(Cl)cc2)CC1)c1ccc(F)cc1 ; O=C(CCCN1CCC(O)(c2ccc(Cl)cc2)CC1)c1ccc(Cl)cc1 ; O=C(CCCN1CCC(O)(c2ccc(Cl)cc2)CC1)c1ccc(Br)cc1 ; O=C(CCCN1CCC(O)(c2ccc(Cl)cc2)CC1)c1ccccc1 ; COc1ccc(C(=O)CCCN2CCC(O)(c3ccc(Cl)cc3)CC2)cc1 ; CC(C)(C)c1ccc(C(=O)CCCN2CCC(O)(c3ccc(Cl)cc3)CC2)cc1]

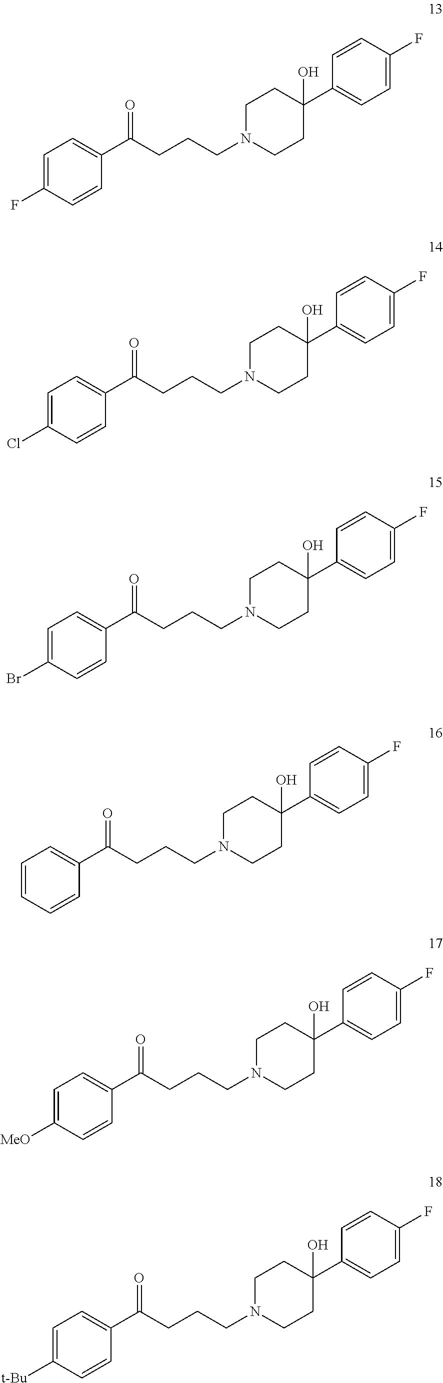 [SMILES: O=C(CCCN1CCC(O)(c2ccc(F)cc2)CC1)c1ccc(F)cc1 ; O=C(CCCN1CCC(O)(c2ccc(F)cc2)CC1)c1ccc(Cl)cc1 ; O=C(CCCN1CCC(O)(c2ccc(F)cc2)CC1)c1ccc(Br)cc1 ; O=C(CCCN1CCC(O)(c2ccc(F)cc2)CC1)c1ccccc1 ; COc1ccc(C(=O)CCCN2CCC(O)(c3ccc(F)cc3)CC2)cc1 ; CC(C)(C)c1ccc(C(=O)CCCN2CCC(O)(c3ccc(F)cc3)CC2)cc1]

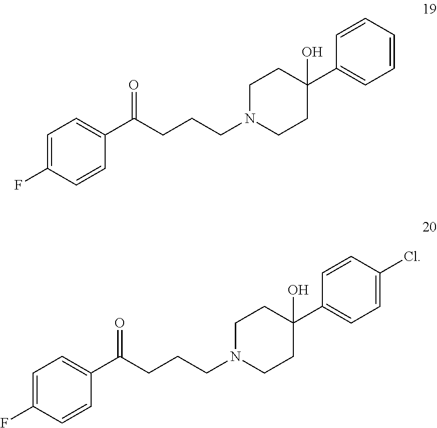 [SMILES: O=C(CCCN1CCC(O)(c2ccccc2)CC1)c1ccc(F)cc1 ; O=C(CCCN1CCC(O)(c2ccc(Cl)cc2)CC1)c1ccc(F)cc1]

Although described above with respect to antipsychotic agents according to Formula I, as will be appreciated by those skilled in the art, the antipsychotic agents are not so limited and may include any other suitable antipsychotic agent or derivative thereof. For example, in some embodiments, other antipsychotic agents which may be combined with the antifungal agent include, but are not limited to:

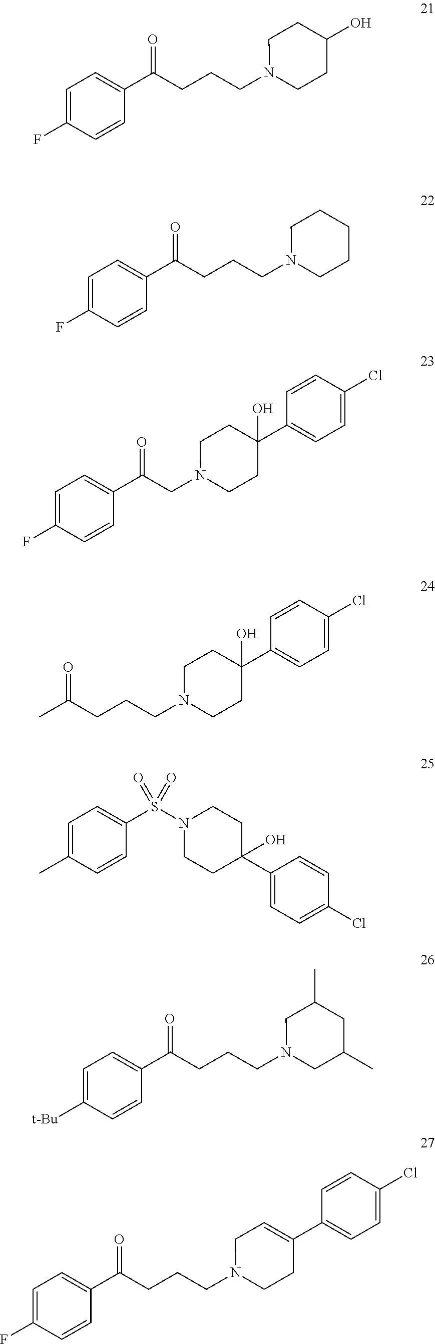

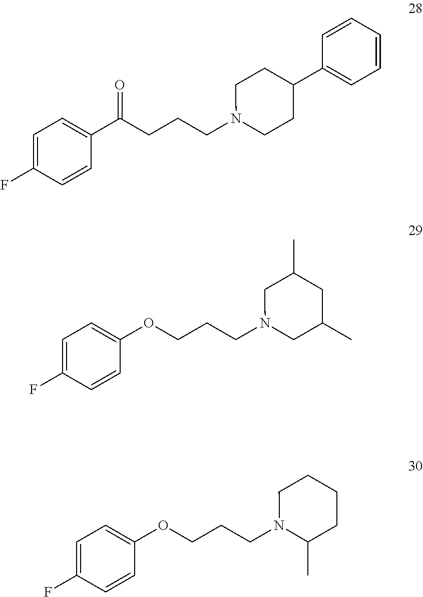 [SMILES: O=C(CCCN1CCC(c2ccccc2)CC1)c1ccc(F)cc1 ; CC1CC(C)CN(CCCOc2ccc(F)cc2)C1 ; CC1CCCCN1CCCOc1ccc(F)cc1]

Additionally or alternatively, the composition may include an antihistamine. For example, in some embodiments, the composition includes an antifungal agent and an antihistamine. In one embodiment, the antihistamine includes terfenadine (TERF), ebastine (EBA), and/or derivatives thereof.

The antihistamine and/or antipsychotic agents disclosed herein may be combined with any suitable antifungal agent. In some embodiments, the antifungal agent is an azole. In some embodiments, the antifungal agent is a non-azole antifungal. For example, suitable antifungal agents include, but are not limited to, one or more of fluconazole (FLC), itraconazole (ITC), ketoconazole (KTC), posaconazole (POS), and voriconazole (VOR), ketoconazolev(KTC), undecylic acid (undecanoic acid), nystatin (NYS), naftifine (NAF), tolnaftate, amorolfine, butenafine (BTF), miconazole (MCZ), econazole, ciclopirox, oxiconazole, sertaconazole, efinaconazole, clotrimazole (CLO), sulconazole, tioconazole, tavaborole, terbinafine (TER), mancozeb, tricyclazole, carbendazim, hexaconazole, propineb, metalaxyl, benomyl (BEN) (Methyl 1-(butylcarbamoyl)-2-benzimidazolecarbamate), difenoconazole, propiconazole (PCZ), kitazin, tebuconazole (TER), tridemorph (TDM), and metconazole (MET). These antifungal agents are also shown below in Table 1.

TABLE 1
Example Antifungal Agents

| Name | Structure |
|---|---|
| Fluconazole (FLC), Itraconazole (ITC), Ketoconazole (KTC), Posaconazole (POS), and Voriconazole (VOR) | A 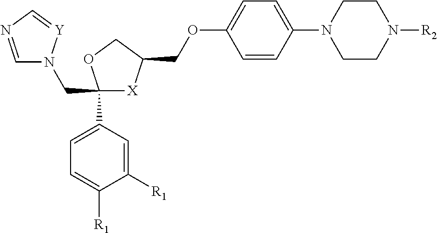 |
| | ITC: R₁ = Cl, X = O, Y = N, |
| | 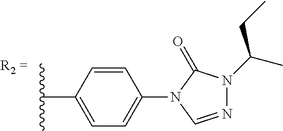 [SMILES: *c1ccc(-n2cnn([C@H](C)CC)c2=O)cc1] |
| | KTC: R₁ = Cl, X = O, Y = CH, R₂ = Ac |
| | POS: R₁ = F, X = [symbol] , Y = N, |
| | 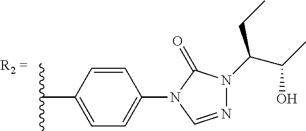 [SMILES: *c1ccc(-n2cnn([C@@H](CC)[C@H](C)O)c2=O)cc1] |
| | 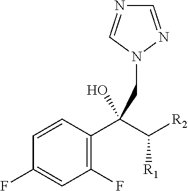 |
| | 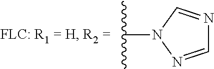 [SMILES: *n1cncn1] |
| | 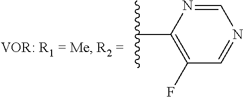 [SMILES: *c1ncncc1F] |
| Ketoconazole (KTC) | 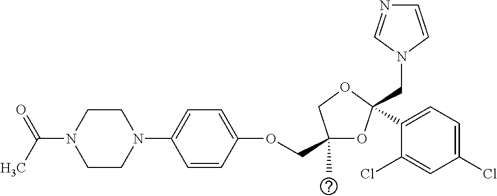 |
| Miconazole (MCZ) | 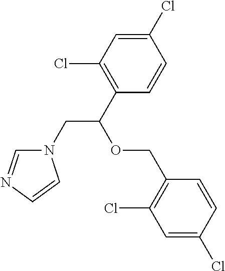 |
| Econazole | 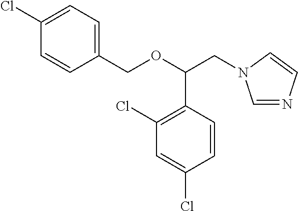 |
| Oxiconazole | 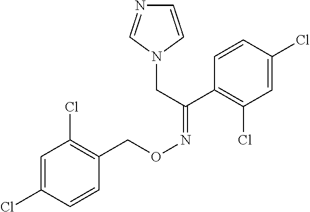 |
| Sertaconazole | 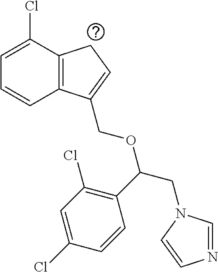 |
| Efinaconazole | 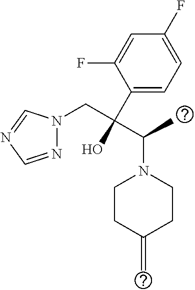 |
| Clotrimazole (CLO) | 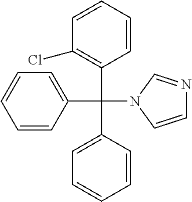 |
| Sulconazole | 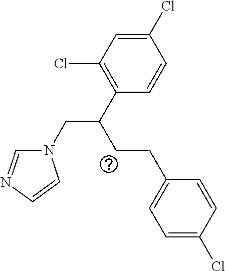 |
| Tioconazole | 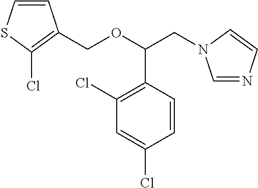 |
| Tricyclazole | 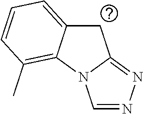 |
| Carbendazim | 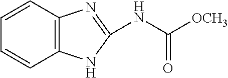 |
| Hexaconazole | 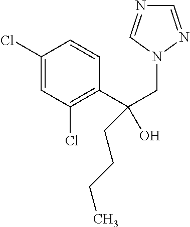 |
| Methyl 1-(butylcarbamoyl)-2- benzimidazolecarbamate (BEN) | 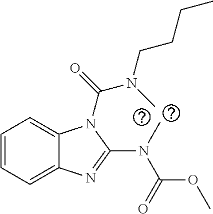 |
| Difenoconazole | 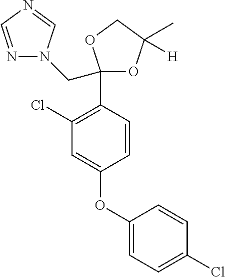 |
| Propiconazole (PCZ) | 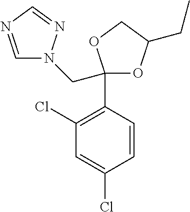 |
| Tebuconazole (TER) | 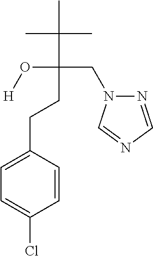 |
| Metconazole (MET) | 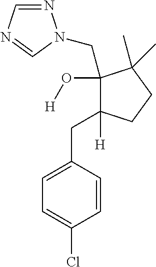 |
| Undecylic acid (undecanoic acid) | 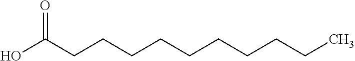 |
| Nystatin (NYS) | 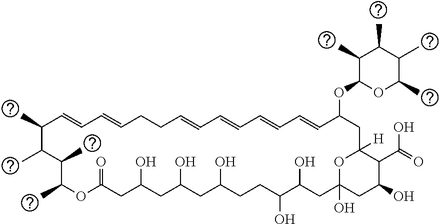 |
| Naftifine (NAF) | 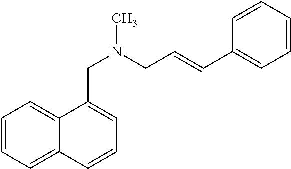 |
| Tolnaftate | 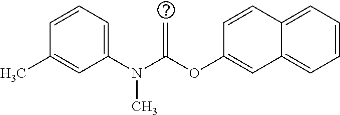 |
| Amorolfine | 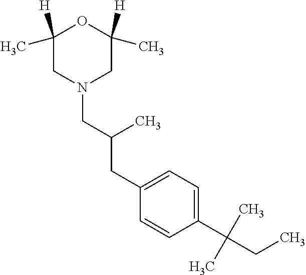 |
| Butenafine (BTF) | 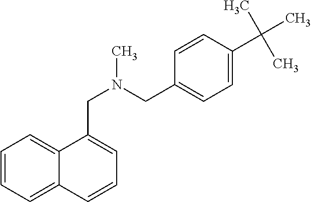 |
| Ciclopirox | 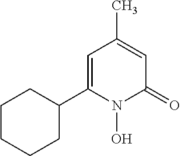 |
| Tavaborole | 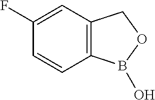 |
| Terbinafine (TER) | 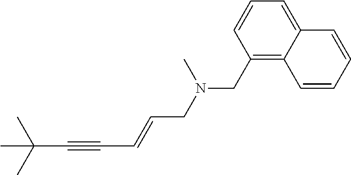 |
| Mancozeb | [symbol] |
| Propineb | 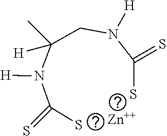 |
| Metalaxyl | 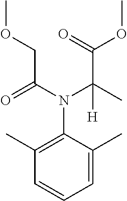 |
| Kitazin | 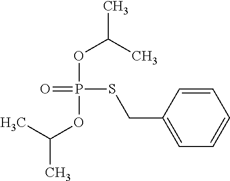 |
| Tridemorph (TDM) | 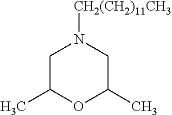 |

[symbol]

indicates data missing or illegible when filed

In some embodiments, the the antipsychotic agents and/or antihistamines alone do not exhibit antifungal activity, but when combined with an antifungal agent they synergistically increase the antifungal properties thereof. As such, in one embodiment, the composition has increased antifungal activity as compared to the antifungal agent alone. In another embodiment, the composition has synergistically increased antifungal activity as compared to the antifungal agent alone. In this regard, the presently-disclosed subject matter is based, in part, on the surprising discovery that certain compounds, including unique compounds identified herein, achieve improved antifungal activity when placed in combination with known antifungal agents. Accordingly, a known antifungal agent can be improved in terms of antifungal efficacy, as well as side-effects, because there is a reduced amount of the antifungal agent needed to achieve an equivalent antifungal activity when placed in combination with compounds disclosed herein.

Thus, the compositions disclosed herein address the inability of certain antifungals to sufficiently inhibit antifungal pathogens, including azole-resistant fungal pathogens, in various fungal infections. Additionally or alternatively, the compositions disclosed herein alleviate side effects, including azole-induced side effects, such as toxicity and drug-drug interactions with concurrently-administered medications, by reducing the amount of azole antifungal required to achieve an equivalent antifungal effect. The compositions also meet the demand for unique and effective antifungal therapies, not only in clinical medicine but also in agriculture, as the incidents of fungal infections keep rising every year and will continue to rise in the future.

In some embodiments of the presently-disclosed subject matter, the compound and the antifungal agent are provided in the composition in a particular ratio. In one embodiment, the particular ratio is selected to enhance or maximize antifungal activity of the composition and/or minimize adverse side effects of the composition while maintaining antifungal activity. In another embodiment, the particular ratio provides an effective amount of the antifungal agent in the composition to provide antifungal activity that is less than the effective amount of the antifungal agent when used alone.

As will be appreciated by those of ordinary skill in the art, the particular range of ratios can vary based on the specific fungal strain and the particular combination of antiviral agent and compound; however, the particular ratio or range for a given situation can be identified in view of the disclosure herein with no more than reasonable routine experimentation. In some embodiments of the presently-disclosed subject matter, the ratio of antifungal agent to compound is between about 1:1 and 1:1100, between about 1:1 and 1:500, between about 1:10 and 1:500, between about 1:1 and 1:300, between about 1:1 and 1:100, between about 1:1 and 1:10, between about 1:1 and 1:5, or any combination, sub-combination, range, or sub-range thereof. For example, in one embodiment, where the antifungal agent is an azole and the compound is an antipsychotic derivative, synergy can be observed with about 1:1 to 1:4 azole:antipsychotic. Other synergistic combinations involved ratios of azole to antipsychotics of about 1:128, 1:512, or 1:1024. With the combination of azoles and compounds that are antihistamine derivatives, synergistic combinations can involve ratios of about 1:10 to 1:340 (azole:antihistamine). Other synergistic combinations involved ratios with more azoles, such as 1:1 to 1:10.

The presently-disclosed subject matter also includes methods of treating a fungal infection. In some embodiments, the method is for the treatment of an infected plant, such as, but not limited to, a plant infected by a plant pathogen. For example, in one embodiment, the method includes administering an effective amount of the composition disclosed herein to a plant in need thereof. In such embodiments, the composition may be provided as a pharmaceutical composition formulated for administration to a plant (e.g., fungicides or agricultural antifungals).

Additionally or alternatively, in some embodiments, the method is for the treatment of an infected animal, such as, but not limited to, an animal infected by an animal pathogen. For example, in one embodiment, the method includes administering an effective amount of the composition disclosed herein to an animal in need thereof. In such embodiments, the composition may be provided as a pharmaceutical composition formulated for administration to an animal subject. In this regard, the composition can include a pharmaceutically-acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

The animal subject of the herein disclosed methods may be a vertebrate, such as a mammal, a fish, a bird, a reptile, or an amphibian. In some embodiments, the animal subject of the herein disclosed methods is a human. Thus, in one embodiment, the method is for the treatment of an infected human. In this regard, the infection can be caused by a human pathogen. Alternatively, in some embodiments, the animal subject of the herein disclosed methods is non-human. Thus, veterinary therapeutic uses are provided in accordance with the presently disclosed subject matter. Accordingly, the presently disclosed subject matter provides for administration to mammals such as humans and non-human primates, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; rabbits, guinea pigs, and rodents. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

Due to the clinical importance of azole antifungals, many research groups have tried to develop new azole antifungal therapies with broader antifungal spectra, reduced undesired drug-drug interactions, and diminished other adverse effects. Besides developing new azole derivatives, exploring various combination therapies that work synergistically with the current azole antifungals has proved fruitful. For instance, various azoles were shown to display synergistic antifungal activity with a series of amphiphilic tobramycin derivatives with azoles inhibiting ergosterol biosynthesis and the tobramycin derivatives proposed to act on the fungal membrane integrity. Sertraline, an antidepressant, was also found to work synergistically with FLC against cryptococcal infections.

The benefits of repurposing existing drugs that have already been approved by the US Food and Drug Administration (FDA) for a new application against fungal infections extend beyond yielding potentially new antifungal therapies. The previous evaluations of the approved drugs from years of clinical studies can also provide valuable information about these drugs, such as their pharmacokinetics, pharmacodynamics, metabolism, and toxicity profiles. In the effort of repurposing existing drugs, several non-antifungal drugs were identified with antifungal activity and were summarized in a previous review article. Haloperidol (trade name Haldol) is an FDA-approved oral antipsycotic that was recently discovered to possess antifungal activity towards a drug-sensitive C. albicans strain (C. albicans SC5314). Bromperidol, a haloperidol derivative also with antipsychotic properties, was reported to kill mycobacteria in a synergistic manner with spectinomycin, further suggesting that this antipsychotic drug may possess antimicrobial properties. Although the cellular antifungal target of haloperidol/bromperidol is still in debate, this antipsychotic drug can potentially be developed into a combination therapy with azoles as a new antifungal strategy.

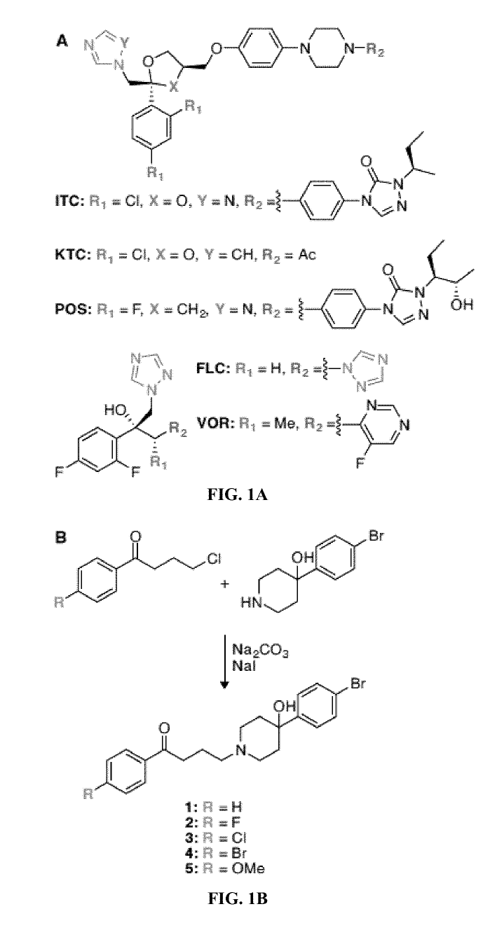

In this Example, the antifungal activity of the combination of common azole antifungals (e.g., fluconazole (FLC), itraconazole (ITC), ketoconazole (KTC), posaconazole (POS), and voriconazole (VOR); FIG. 1A) with bromperidol (2) and its derivatives (1 and 3-5) (FIG. 1B) was evaluated against nine fungal pathogens. The fungal pathogens included seven C. albicans, one non-albicans Candida (C. glabrata), and one filamentous fungus (A. terreus), each of which presents distinct biology and a complex resistance profile.

After assessing the synergistic effect between the azole antifungals and compounds 1-5, time-kill assays were performed to evaluate this antifungal strategy in a time- and dose-dependent manner. Moreover, the combination of bromperidol (2) and selected azole antifungals was evaluated for its ability to disrupt yeast biofilm in a representative C. albicans strain. Finally, mammalian cytotoxicity assay were performed with three selected mammalian cell lines in order to estimate the mammalian cytotoxicity exerted by the combinations of compounds 1-5 and azole antifungals. As a result, this Example demonstrates synergistic combinations of azoles and bromperidol derivatives in the treatment of various fungal infections.

### Results and Discussions

Chemical Synthesis of Bromperidol and its Derivatives

The bromperidol series compounds (1-5) were synthesized by a reaction of 4-(4-bromophenyl)-4-hydroxypiperidine with different 4-chlorobutyrophenone derivatives in the presence of sodium iodide and sodium carbonate (FIG. 1B). The substituents at the R position of the five compounds varied in size and included a hydrogen atom, halogens (e.g., fluorine, chlorine, and bromine), and a methoxy group.

Antifungal Synergy of the Combinations of Azole Antifungals and Bromperidol Series Compounds 1-5 by Checkerboard Assays

Prior to evaluating the combinational antifungal effect of azoles and compounds 1-5, the minimum inhibitory concentration (MIC) values of each drug were determined against a variety of fungal pathogens to examine their innate antifungal effect and better gauge for an appropriate concentration range to use for the following checkerboard assay (TABLE 2, displayed as MICₐₗₒₙₑ). Two representative C. albicans strains (strains B and F) were selected first (TABLE 2). C. albicans ATCC 10231 (strain B) is sensitive to most azoles whereas C. albicans ATCC 64124 (strain F) displays resistance to most azoles tested. The MIC values of the azole antifungals against some fungal strains determined in this Example were in agreement with previously reported values. Furthermore, no antifungal effect was observed for compounds 1-5 when tested alone.

The synergistic antifungal effects of the five azoles and compound 1-5 in combination were then tested by checkerboard assays and calculated the fractional inhibitory concentration index (FICI) against strains B and F (TABLE 2). The FICI cutoff values for determining synergy are: synergistic (SYN) if FICI≤0.5, additive (ADD) if 0.5<FICI≤4, antagonistic (ANT) if FICI>4. In some cases where the FICI was >0.5, however, a significant decrease in the MIC values of at least one of the drugs in the combinations was observed. In such cases, partial synergy (pSYN) as 0.5<FICI≤0.75 was further defined (indicating that both drugs showed reduction in MIC values and one drug showed two-fold reduction in MIC value), and strong additive effect (ADD*) where one drug showed two-fold reduction in MIC value. Defining these two categories was deemed necessary, as with these combinations, a low amount of azoles could still be used in combination to achieve a similar antifungal effect as using high concentrations of azoles alone, which would alleviate azole-induced toxicity and side effects.

One thing to note is that due to the resistant nature of some fungal strains and the insufficient antifungal activity of the tested drugs, the instant inventors were unable to achieve full inhibition of fungal growth with some drugs, therefore, resulting in unbound MIC values such as >32 &g/mL for most azoles and >128, >64, and >32 μg/mL for most bromperidol derivatives. In these cases, the MIC values were considered to be 32 μg/mL for the azoles and 128, 64, and 32 μg/mL for compounds 1-5, respectively, in order to calculate a bound FICI value. However, this approximation would produce overestimated FICI values that are higher than the true FICI values if the real MIC values could be determined. Hence, the amount of synergy observed in this study (both in terms of the FICI value for each combination and in terms of the percentage of combinations with synergy) would be an underestimation of the real potential synergy.

Of the 25 combinations tested against strains B and F in the first round, six combinations (24% of all combinations) were found to be synergistic with FICI values ranging from 0.13 to 0.5 against the azole-resistant strain F. The best combination with the lowest FICI value of 0.13 observed was compound 5 and VOR, which showed decrease of MICₐₗₒₙₑ of azole and compound 5 from >32 and >128 μg/mL to 2 and 8 μg/mL (16-fold reduction in MIC values for both drugs), respectively. The second best combination discovered with an FICI value of 0.16 was with compound 2 and POS, which showed decrease of MICₐₗₒₙₑ of azole and compound 2 from >32 and 128 μg/mL to 4 (8-fold MIC reduction) and 4 μg/mL (32-fold MIC reduction), respectively.

These cases demonstrated that the combinations of azoles and bromperidol derivatives could synergistically inhibit the growth of an azole-resistant C. albicans that otherwise would not have responded to high concentrations of either drugs given in the assay. Amongst the six combinations that display synergy, four of them (compound 1 with POS, compound 2 with POS or VOR, and compound 3 with POS) were also found to display partial synergy (0.5<FICI≤0.75) against strain B. Moreover, partial synergy was also observed against both strains B and F for the following combinations: compound 1 with VOR, compound 2 with ITC or KTC, compound 3 with KTC or VOR, and compound 4 with KTC. For example, for the combination of compound 2 and ITC against strain F (FICI=0.56), inhibition of fungal growth could be achieved with 2 &g/mL of ITC (16-fold MIC reduction) and 64 &g/mL of compound 2 (1-fold MIC reduction). Partial synergy was also detected for compound 4 with VOR against strain F as well as for compound 5 with VOR against strain B. With the FICI values calculated in this study likely to be overestimated as explained above, these combinations with partial synergy also have great potentials for being developed into synergistic antifungal therapies.

Besides the combinations found to exert synergistic or partial synergistic effect between the azole antifungals and our bromperidol derivatives, one combination was also found to display strong additive effect, which is the combination of VOR and compound 5 against strain F. An FICI value of 1.25 indicated that this combination displayed additive effect. However, looking at the MIC values of each drug alone and in combination, a significant decrease (4-fold reduction) was found in the MIC of VOR in combination compared to that of VOR alone even though the MIC value of compound 5 showed no further decrease from 128 &g/mL. This suggested that the addition of compound 5 significantly reduced the amount of VOR required to inhibit fungal growth, alleviating the toxicity and other undesired side effects of azole antifungals. The rest of the combinations (11 out of 25 combinations) displayed weak additive effect against both strains B and F with FICI values ranging from 1.00 to 2.00. No antagonism was found in any combinations tested in this Example.

When all FICI values for the 25 combinations in TABLE 3 were collected and analyzed in a heat map style table, it became clear that all synergistic combinations discovered so far involve either POS or VOR. Of the five combinations involving POS, 100% displayed synergy against the azole-resistant C. albicans strain F, and 60% displayed partial synergy against strain B. Of all the combinations involving VOR, only one combination (that with compound 2) was found to be synergistic against strain F. Meanwhile, four combinations showed partial synergy against strains B (compounds 1-3 and 5) and F (1, 3, and 4). This demonstrated that the combination of azoles and bromperidol derivatives showed better results with the more resistant fungal strain F. Besides POS and VOR, it was found that three combinations, compounds 2-4 with KTC, showed partial synergy against both fungal strains, and one combination, compound 2 with ITC, displayed partial synergy against both fungal strains. All the combinations involving FLC only displayed weak additive effect. This suggested that POS and VOR might be the best candidates for developing combination therapy with compounds 1-5.

When looking at the five synergistic combinations of POS and compounds 1-5 against strain F, smaller R substituents in the compounds appeared to correlate with lower FICI values. The FICI values increased as the size of the R substituent increased from fluorine to bromine in compounds 2-4. This may indicate that the small size of a fluorine atom as the R substituent in the compounds may be optimal for interacting with its cellular target in fungal cells, and increase in the size of the R substituent may cause loss of engagement with the target due to steric hindrance. This postulation could also be observed by looking at the overall number of combinations that show synergistic tendency (synergy or partial synergy) amongst all 25 combinations tested in the first round. Compound 2, with a fluorine substituent, showed the highest number of combinations (four out of five combinations) with synergistic tendencies. This number decreases as the size of the R substituent increases (from fluorine to bromine in compounds 2-4). Compound 5, with a methoxy substituent, showed the best synergistic effect in combination with POS with the lowest FICI value identified in this Example so far. Although methoxy group is the largest R group amongst all five compounds in this Example, the presence of an oxygen atom and the lowest FICI value of 0.13 might suggest either potential hydrogen bonding involved in the interaction of compound 5 with its target or methoxy group, as a strong electron donating group, increased the π-π intraction of the connected phenyl ring with the target protein/enzyme.

With POS and VOR appearing to be the best azoles to develop combinational antifungal therapy with compounds 1-5, the fungal collection was further expanded and the combination of either POS or VOR and compounds 1-5 was tested against seven additional strains in order to better assess the potential synergy against a wide variety of pathogenic fungi with distinct biological features (TABLE 4). Amongst these seven additional fungal strains were five extra C. albicans strains, one non-albicans Candida strain (C. glabrata, strain II), and one filamentous fungus (A. terreus, strain 1). All of these fungi are resistant to POS and VOR, except for A. terreus, which is sensitive to both of these azoles. The checkerboard assay results for POS and VOR in combination with compounds 1-5 against strain B and F from TABLE 2 were also listed in TABLE 4 for easy comparison.

Of the ten combinations listed in TABLE 4, each against nine fungal strains, seven combinations were found to display synergistic interactions. Compound 2 in combination with POS or VOR showed synergy against strains F, G, and I or F and G, respectively. Compound 3 with either POS or VOR exhibited synergistic interactions against strain F. The other compounds, 1, 4, and 5, all demonstrated synergistic interactions when combined with POS against strains F. Compounds 4 and 5 also were found to be synergistic with POS against strains H and I, respectively. Furthermore, all ten combinations exhibited partial synergy against at least one fungal strain, four of which also displayed strong additive effect against a variety of fungal strains (compound 1 with POS or VOR, compound 3 with POS, and compound 5 with VOR).

The best combination appeared to be compound 2 and POS, as this combination exhibited synergistic interactions against three fungal strains and partial synergy against four more out of the nine fungal strains we tested. No combinations were discovered to have antagonistic interactions (FICI>4). Additionally, it was found that more combinations involving POS showed synergistic or partially synergistic effect compared to the combinations involving VOR. For instance, all five combinations involving POS displayed synergy, whereas only two out of five combinations involving VOR tested exhibited synergistic interactions. These data demonstrated better synergy from developing combination antifungal therapy with POS and bromperidol series derivatives.

In addition to the various C. albicans strains, synergy was also observed in the non-albicans Candida, C. glabrata, and the filamentous fungus, A. terreus. Amongst the ten combinations against C. glabrata (strain H), one combination (compound 4 with POS) was found to display strong synergy, four combinations (compounds 1, 2, and 5 with POS as well as compound 2 with VOR) were found to display partial synergy, and one combination (compound 1 with VOR) was found to display strong additive effect. Amongst the ten combinations against A. terreus (strain I), two combinations (compounds 2 or 5 with POS) were found to be synergistic, one combination was found to display partial synergy, and one combination was found to display strong additive effect. Overall, these data further suggested that combining azoles and compounds 1-5 as an antifungal strategy would be effective against a variety of fungal pathogens and benefit patients suffering from different fungal infections.

Time-Dependent Antifungal Activity of the Combination of Azole Antifungals and Bromperidol Series Compounds

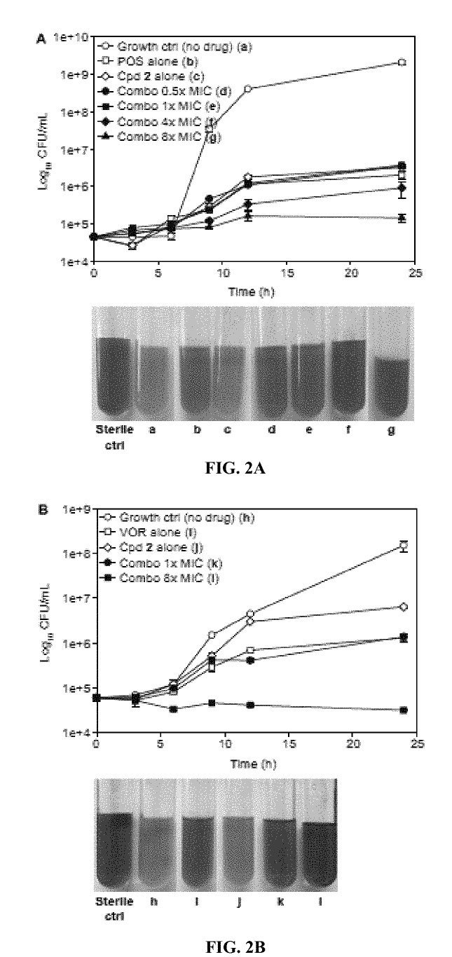

In order to confirm the synergistic interaction between the bromperidol series compounds and azole antifungals, time-kill assays were performed against strain F for two representative combinations (compound 2 and either POS or VOR) that displayed great synergy in previous checkerboard assays (FIGS. 2A-B). In these time-kill assays, the antifungal effect of each drug alone (POS, VOR, or compound 2) as well as the two combinations (POS or VOR with compound 2) were evaluated at various concentrations (0.5×-8×MIC_{combo} of each drug). Overall, the combinations of POS or VOR and compound 2 showed fungistatic effect at 8×MIC_{combo} concentrations. The CFU/mL values did not differentiate amongst various samples until after 6 h. In the first combination tested (POS+compound 2), significant growth was seen in the growth control (increase in CFU/mL by 4 order of magnitude). Meanwhile, the POS or compound 2 alone samples showed slight inhibition of fungal growth similar to that of the 0.5× and 1×MIC_{combo} samples (increase in CFU/mL by about 1 order of magnitude). The combination sample with 4×MIC_{combo} showed stronger inhibition of fungal growth compared to the alone samples as well as the combination samples with less drugs. The combination sample with 8×MIC_{combo} showed complete inhibition of fungal growth and the CFU/mL value for this sample remained around 1×10⁵ CFU/mL throughout 24 h. The growth of the fungus in each sample was further assessed by the addition of resazurin after the 24-h time point, which can be metabolized by live fungal cells and turns the solution from blue to pink. The combination of VOR and compound 2 showed a similar profile to that of the combination of POS and compound 2, except that the combination sample with 8×MIC_{combo} showed slight reduction in CFU/mL by about 1 log₁₀ unit over 24 h.

Biofilm Disruption with the Combination of Representative Azole Antifungals and Bromperidol Series Compounds

Recent discoveries suggest that fungi in biofilms display drastically different biology and antimicrobial susceptibility when compared to free living (planktonic) fungal cells. Biofilm-forming (sessile) fungal cells are protected by extracellular matrices and display increased resistance against a variety of antifungal agents. Using a water soluble metabolic dye, XTT (2,3-bis(2-methoxy-4-nitro-5-sulfo-phenyl)-2H-tetrazolium-5-carboxanilide), the metabolic activity of biofilm (sessile cells) can be measured in checkerboard format assays. In this study, two representative combinations (POS or VOR with compound 2) were selected against strain F, and the antifungal effect that these combinations have on sessile cells was evaluated (TABLE 5). It was reported that the sessile cells of strain F are highly resistant to all drugs tested, including POS, VOR, and compound 2 alone with sessile MIC (SMIC) values of >32, >32, and >128 μg/mL, respectively. When tested in combination, both combinations showed strong additive antifungal effect with FICI values of 1.02. Even though the SMIC value of compound 2 in combination did not decrease by much compared to that alone, the SMIC values of POS or VOR both decrease from >32 to 0.5 μg/mL, which further suggested much stronger resistance of the sessile fungal cells compared to planktonic cells as well as the great antifungal potential of the combination of azoles and bromperidol series compounds.

TABLE 5
Inhibition of biofilm formation with
azoles and compound 2 combinations.

| | | | SMIC alone | | SMIC combo | | | |
|---|---|---|---|---|---|---|---|---|
| | | | (μg/mL) | | (μg/mL) | | | |
| Cpd | Azole | Strain | Azole | Cpd | Azole | Cpd | FICI | Interp. |
| 2 | POS | F | >32 | >128 | 0.5 | 128 | 1.02 | ADD* |
| | VOR | F | >32 | >128 | 0.5 | 128 | 1.02 | ADD* |

Strain F - C. albicans ATCC 64124.
The FICI cutoff values for determining synergy are: synergistic (SYN) if FICI ≤ 0.5, additive (ADD) if 0.5 < FICI ≤ 4, antagonistic (ANT) if FICI > 4.
Since the highest concentration of compound 2 or azole alone did not achieve complete growth inhibition. the MICₐₗₒₙₑ value used in the FICI calculation is the highest concentration tested of compound 2 or azole drugs.
ADD* indicates strong additive effect (one drug showed ≥2-fold reduction in MIC value).

Mammalian Cytotoxicity of the Combination of Representative Azole Antifungals and Bromperidol Series Compounds

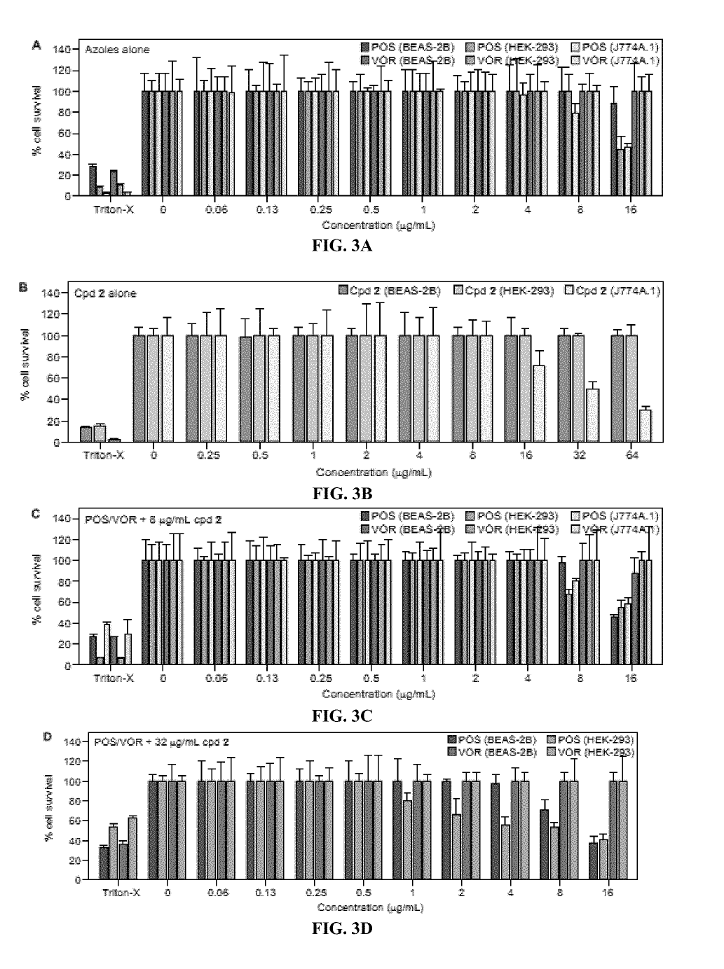

In addition to assessing the time-dependent killing and disruption of fungal biofilm, the mammalian cytotoxicity of azole antifungals and the bromperidol compounds alone and in combination was evaluated (FIGS. 3A-D). In order to gain a better understanding of the toxicity profile towards different mammalian cells, representative azoles (POS and VOR) and compound 2 were evaluated against three different mammalian cell lines, including human bronchial epithelial cells BEAS-2B, human kidney epithelial cells HEK-293, and the murine macrophage J774A.1. Please note that as many xenobiotics stimulate cell growth instead of exerting toxicity at sub-IC₅₀ concentrations, resulting in >100% cell survival in the treatment groups, these >100% cell survival data was considered as no observed toxicity and expressed as 100% cell survival.

When testing the azole antifungals alone (FIG. 3A), no cytotoxic effect was observed up to 4 μg/mL. At 8 μg/mL, 80±9% cell survival was observed with J774A.1 cells, and at 16 μg/mL, around 45±12% and 46±3% cell survival was observed with POS-treated HEK-293 and J774A.1 cells, respectively. No cytotoxic effect was observed in any VOR-treated cell lines at any concentrations tested. With BEAS-2B and HEK-293 cells being more robust cell lines than J774A.1, it was not surprising to see the absence of toxicity in these two cell lines compared to that in J774A.1 cells, for macrophages are often short-lived and fragile within the human body.

The toxicity of compound 2 was then assessed alone as a representative of the bromperidol series derivatives (FIG. 3B). No toxicity was found against BEAS-2B and HEK-293 cells at 64 μg/mL. Compound 2 alone exerted toxicity against J774A.1 cells and showed 72±14%, 50±7% and 30±4% cell survival with 16, 32, and 64 μg/mL compound 2, respectively. These findings suggested that the bromperidol series compounds had much better toxicity profiles against various mammalian cells and that the strategy of using our bromperidol series compounds in combination with azole antifungals can effectively help alleviate azole-induced toxicity by reducing the amount of azole antifungals required in treatment.

Since 8 μg/mL compound 2 was the highest concentration at which no cytotoxicity was observed with all three cell lines, the cytotoxicity assay of POS and VOR was performed against all three cell lines with 8 μg/mL compound 2 supplemented in the media (FIG. 3C). Similar overall cytotoxic effect as in the azoles alone samples was observed. The percent cell survivals were slightly lower at 8 or 16 μg/mL POS in combination compared to that of POS alone. Meanwhile, no toxicity was observed at 16 μg/mL VOR in combination with 8 μg/mL compound 2. As J774A.1 is the most fragile cell line tested, the combination of azoles and a higher concentration of compound 2 (32 μg/mL) was also tested against the two epithelial cell lines (BEAS-2B and HEK-293 cells) (FIG. 3D). Of the combination of POS and compound 2 against BEAS-2B cells, 71±11% and 38±6% cell survival was observed at 8 and 16 μg/mL POS. However, the combination toxicity of POS and 32 μg/mL compound 2 was more prominent against HEK-293 cells where decreased cell survival from 80±8% to 40±6% was observed as the concentration of POS increased from 1 to 16 μg/mL. With 32 μg/mL compound 2, there was still no toxicity noted against either cell lines at any concentration of VOR, which proved the better toxicity profile of VOR compared to that of POS.

Judging from the results from cytotoxicity assays, POS seemed to be toxic to mammalian cells. Thus, developing combinational antifungal therapy that involves less POS and more of the nontoxic bromperidol compounds seemed to be a reasonable approach to alleviate azole-induced toxicity and other related side effects. In addition to the great potential of synergy between POS and bromperidol compounds, VOR also has great potential to be developed into combinational antifungal therapies due to its nontoxic nature.

Haloperidol/bromperidol, originally antipsychotic drugs, act on dopamine D2 receptors, which is a G protein-coupled receptor with p-glycoprotein properties. However, as newly discovered antifungal candidates, their cellular target in fungal cells remained elusive. Although some reports indicated that haloperidol might target the biosynthesis and metabolism of amino acids or fungal morphogenesis and hyphal formation in fungal pathogens, others pointed out that the multidrug-resistant transporter (MDR1), a p-glycoprotein, is more likely to be the antifungal target of this antipsychotic drug. Inhibition of MDR1, an active transporter/efflux pump that contributes to efflux-related azole resistance, can further sensitize fungal pathogens to azole antifungals and prolong their antifungal effect. The bromperidol series compounds presented in this study, due to structural similarity, are also likely to exert their antifungal properties in the same way. This theory could also explain why the bromperidol series compounds possessed no antifungal activity by themselves, but could produce great antifungal synergy in combination with various azoles. A recent study reported synergistic antifungal effect of FLC and VOR in combination with haloperidol as an MDR1 inhibitor against two Malassezia strains, which also demonstrated the feasibility and benefits of developing new antifungal therapies with the combination of haloperidol or its derivatives and azole antifungals.

### CONCLUSIONS

In this Example, the antifungal effect of bromperidol and four of its derivatives in combination with five clinically relevant azole antifungals were evaluated against a wide variety of pathogenic fungi. From our extensive evaluation of the combinational antifungal effect between the two classes of compounds by checkerboard, time-kill, and biofilm disruption assays, a wide range of combinational effects were observed, ranging from synergistic to weak additive effect. A considerable portion of the combinations tested in this study displayed synergy or partial synergy. It was also found that POS displayed synergy in more combinations with bromperidol series compounds than VOR did. However, the cytotoxicity evaluation suggested combination therapy with VOR might have superior mammalian cytotoxicity profiles. As mentioned above, the FICI calculated in this study are likely to be higher than the true FICI values due to the unbound MIC values. Therefore, the potential synergy and the number of combinations showing synergistic effects are also likely to be underestimated. Even though the exact cellular target by which the bromperidol series compounds exert antifungal activity when combined with azole antifungals remains unclear, the results suggested that using these bromperidol derivatives in combination with clinically relevant azoles can synergistically inhibit fungal growth and effectively reduced the amount of azoles required to achieve an equivalent antifungal effect, and therefore, alleviate the toxicity and side effects resulted from administering high concentrations of azole antifungals.

### Example 2

This Example describes the chemistry and synthesis of compounds 1-5 in Example 1.

1. Chemistry

1.1. Materials and Instrumentation for Chemistry.

All reagents were bought from commercial sources and no further purification was performed before usage. TLC analyses were performed on 0.25-mm thick silica gel plates (pre-coated on glass) with fluorescent indicator UV254, and were visualized by UV or charring in a KMnO₄ stain. ¹H and ¹³C NMR spectra were recorded on a 400 MHz NMR spectrometer (VARIAN INOVA) using CDCl₃, CD₃OD, or (CD₃)₂SO as solvents. Chemical shifts were reported in parts per million (ppm) and were referenced to residual solvent peaks. All reactions were carried out under nitrogen gas with all yields reported representing isolated yields. All compounds were characterized by ¹H and ¹³C NMR as well as mass spectrometry. Although compound 2 has been reported in the literature, its characterization has not been published in details. NMR spectra confirm that all compounds are ≥95% pure. Further confirmation of compound purity was obtained by RP-HPLC, which was performed on an Agilent Technologies 1260 Infinity HPLC system by using the following general method 1: Flow rate=1 mL/min; Δ=254 nm; column=Vydac 201SP™ C18, 250×4.6 mm, 90A 5 μm; Eluents: A=H₂O+0.1% TFA, B=MeCN; gradient profile: starting from 5% B, increasing from 5% B to 100% B over 20 min, holding at 100% B from 20-27 min, decreasing from 100% B to 5% B from 27-30 min. The HPLC column was equilibrated with 5% B for 15 min prior to each injection.

1.2. Synthesis and Characterization of Compounds Used in this Study.

General Procedure for the Amination Reaction (e.g., Synthesis of Compound 1).

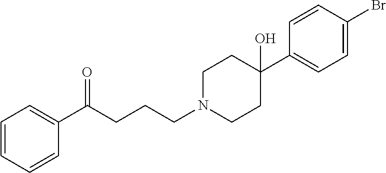 [SMILES: O=C(CCCN1CCC(O)(c2ccc(Br)cc2)CC1)c1ccccc1]

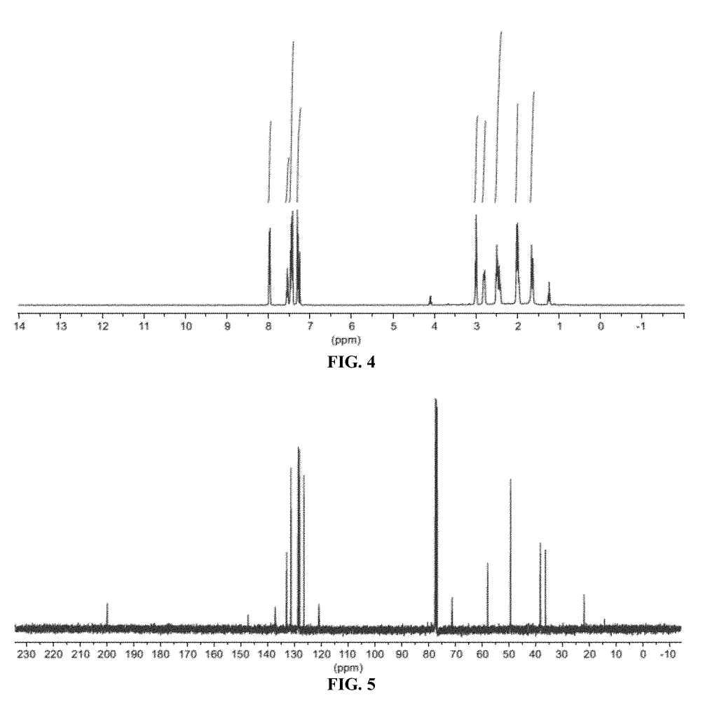

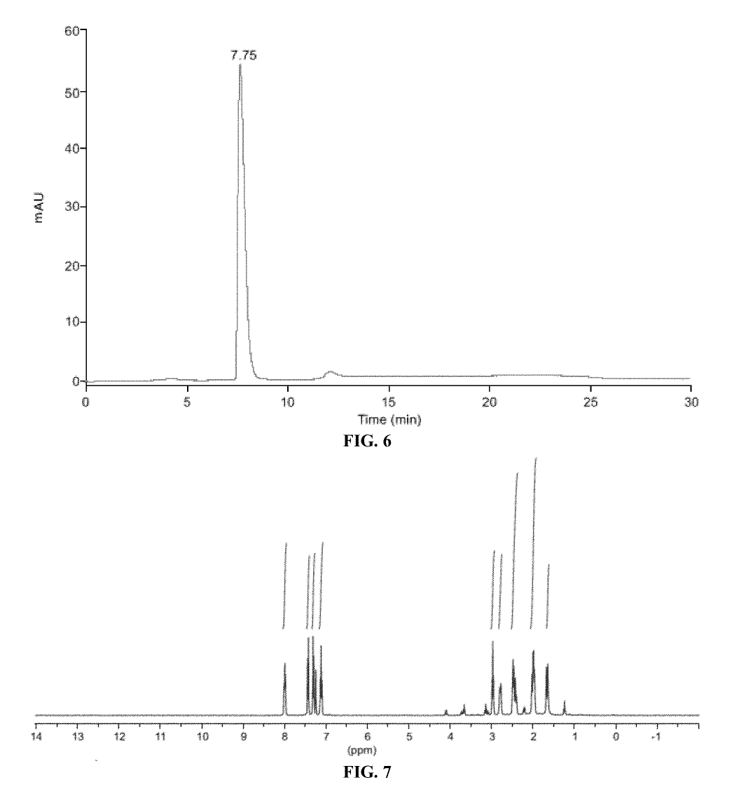

Compound 1 was prepared following a previously published protocol for a similar molecule.¹ Sodium iodide (0.039 g, 0.260 mmol) and sodium carbonate (0.050 g, 0.472 mmol) were added to a stirred mixture of 4-chlorobutyrophenone (0.043 g, 0.236 mmol) and 4-(4-bromophenyl)-4-hydroxypiperidine (0.060 g, 0.236 mmol) in MeCN (2 mL). The reaction mixture was refluxed for 12 h. The mixture was diluted with H₂O, and extracted with CH₂Cl₂ (3×10 mL), dried over MgSO₄, and concentrated under reduced pressure. The residue was purified by column chromatography (SiO₂, MeOH:EtOAc/1:9, R_{f}0.22), to give compound 1 (0.016 g, 17%) as a white solid: ¹H NMR (400 MHz, CDCl₃, FIG. 4) δ 7.97 (d, J=8.0 Hz, 2H), 7.54 (t, J=7.2 Hz, 1H), 7.46 (d, J=7.2 Hz, 2H), 7.42 (d, J=8.0 Hz, 2H), 7.29 (d, J=8.0 Hz, 2H), 3.00 (t, J=6.8 Hz, 2H), 2.81 (m, 2H), 2.50 (t, J=6.8 Hz, 2H), 2.44 (t, J=12.0 Hz, 2H), 2.04-1.96 (m, 2H), 2.00 (p, J=6.8 Hz, 2H), 1.66-1.63 (m, 3H); ¹³C NMR (100 MHz, CDCl₃, FIG. 5) δ 199.9, 147.3, 137.2, 132.9, 131.3, 128.5, 128.1, 126.4, 120.8, 71.1, 57.8, 49.2, 38.2, 36.2, 21.8; LRMS m/z calcd for C₂₁H₂₅BrNO₂[M+H]⁺: 402.1; found 402.7. Purity of the compound was further confirmed by RP-HPLC by using method 1: Rₜ=7.75 min (97% pure; FIG. 6)

Synthesis of Compound 2.

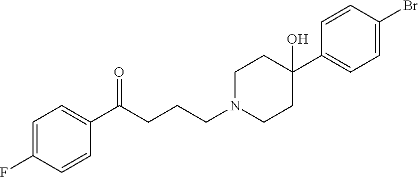 [SMILES: O=C(CCCN1CCC(O)(c2ccc(Br)cc2)CC1)c1ccc(F)cc1]

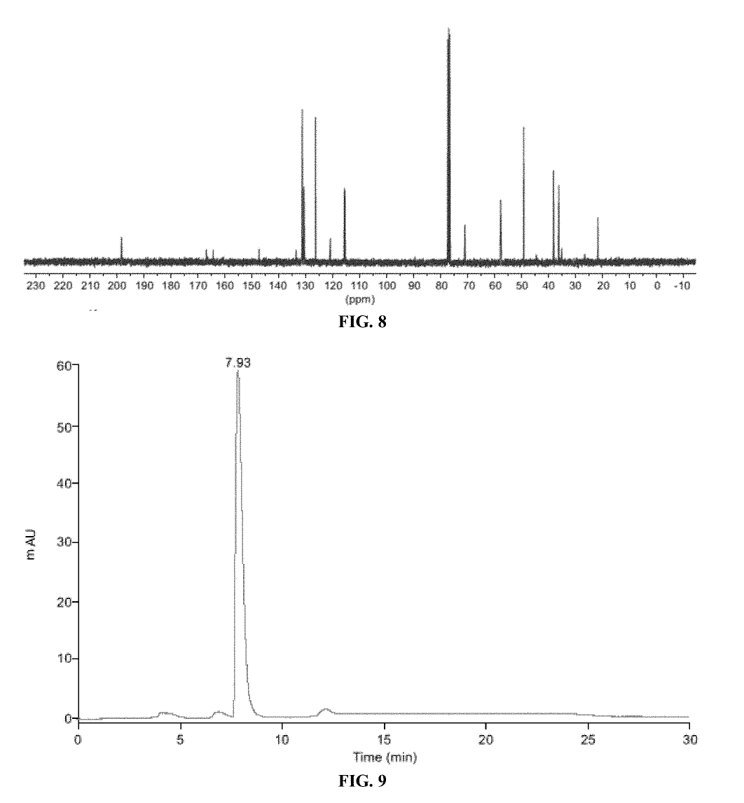

Following the general procedure described for the synthesis of compound 1, sodium iodide (0.039 g, 0.260 mmol), sodium carbonate (0.050 g, 0.472 mmol), 4′-fluoro-4-chlorobutyrophenone (0.047 g, 0.236 mmol), and 4-(4-bromophenyl)-4-hydroxypiperidine (0.060 g, 0.236 mmol) in MeCN (2 mL) were used to afford know compound 2 (also known as bromperidol)² (0.024 g, 24%, R_{f}0.15 in MeOH:EtOAc/1:9) as a white solid: ¹H NMR (400 MHz, CDCl₃, FIG. 7) δ 7.99 (dd, J=8.4, 6.0 Hz, 2H), 7.43 (d, J=8.4 Hz, 2H), 7.30 (d, J=8.4 Hz, 2H), 7.11 (app. t, J=8.4 Hz, 2H), 2.97 (t, J=6.8 Hz, 2H), 2.79 (m, 2H), 2.48 (t, J=6.8 Hz, 2H), 2.42 (t, J=11.2 Hz, 2H), 2.04-1.94 (m, 2H), 1.98 (p, J=6.8 Hz, 2H), 1.67-1.64 (m, 3H); ¹³C NMR (100 MHz, CDCl₃, FIG. 8) δ 198.3, 166.9, 164.3, 147.3, 133.61, 133.58, 131.3, 130.7, 130.6, 126.4, 120.9, 115.7, 115.5, 71.1, 57.8, 49.3, 38.2, 36.2, 21.8; LRMS m/z calcd for _{C21H24BrFNO2} [M+H]⁺: 420.1; found 420.7. Purity of the compound was further confirmed by RP-HPLC by using method 1: Rₜ=7.93 min (95% pure; FIG. 9).

Synthesis of Compound 3.

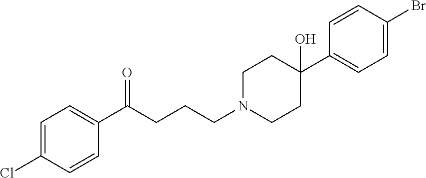 [SMILES: O=C(CCCN1CCC(O)(c2ccc(Br)cc2)CC1)c1ccc(Cl)cc1]

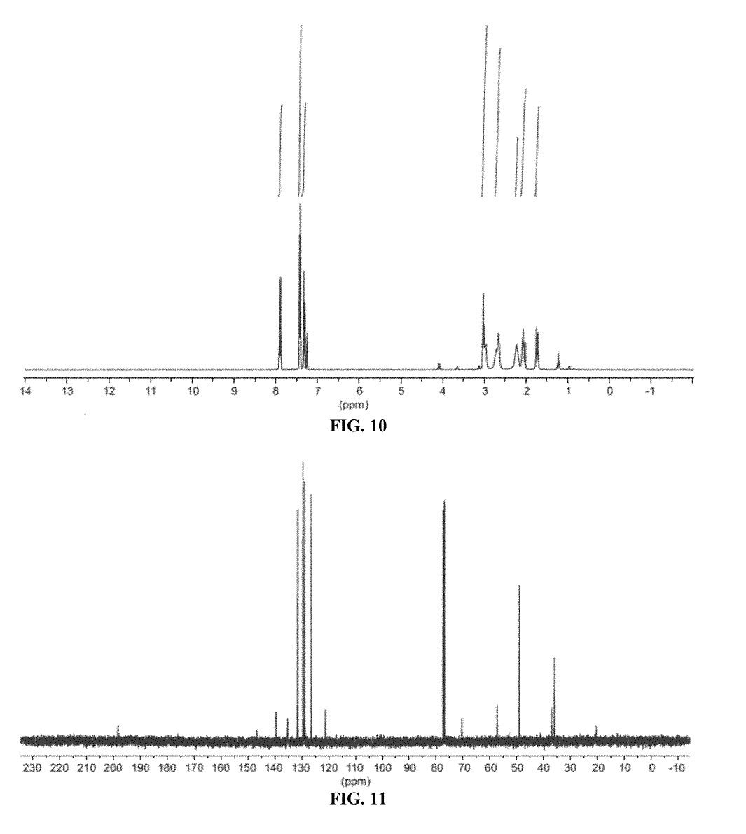

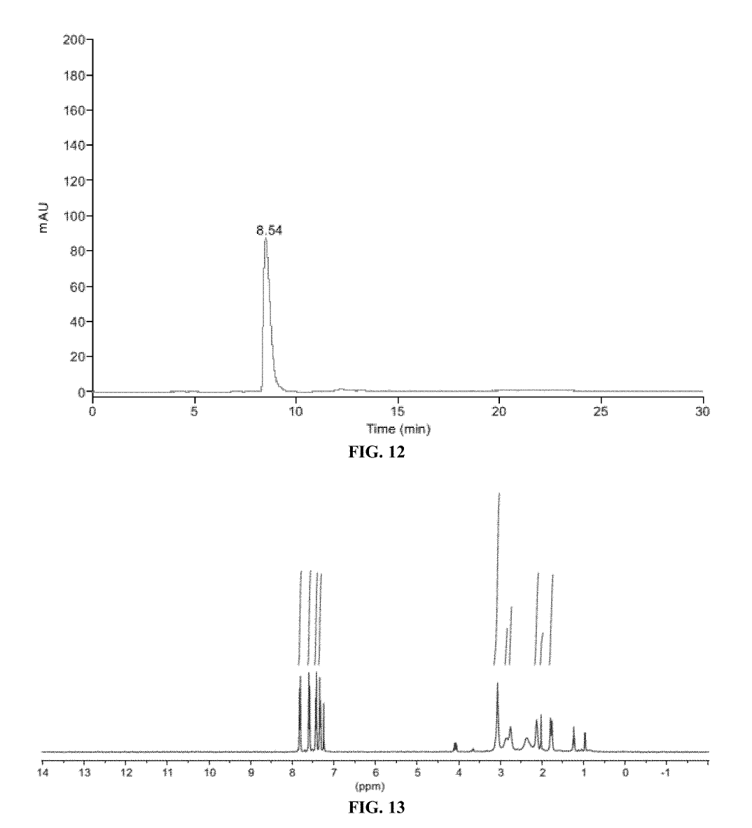

Following the general procedure described for the synthesis of compound 1, sodium iodide (0.039 g, 0.260 mmol), sodium carbonate (0.050 g, 0.472 mmol), 4′-chloro-4-chlorobutyrophenone (0.051 g, 0.236 mmol), and 4-(4-bromophenyl)-4-hydroxypiperidine (0.060 g, 0.236 mmol) in MeCN (2 mL) were used to afford compound 3 (0.024 g, 23%, R_{f}0.21 in MeOH:EtOAc/1:9) as a white solid: ¹H NMR (400 MHz, CDCl₃, FIG. 10) δ 7.89 (d, J=8.4 Hz, 2H), 7.42 (d, J=8.4 Hz, 4H), 7.31 (d, J=8.4 Hz, 2H), 3.02 (t, J=6.8 Hz, 2H), 2.97 (m, 2H), 2.77-2.68 (m, 2H), 2.66 (t, J=6.8 Hz, 2H), 2.28-2.14 (m, 3H), 2.07 (p, J=6.8 Hz, 2H), 1.74 (m, 2H); ¹³C NMR (100 MHz, CDCl₃, FIG. 11) δ 198.1, 146.6, 139.5, 135.1, 131.4, 129.5, 128.9, 126.4, 121.1, 70.4, 57.3, 49.1, 37.1, 36.0, 20.5; LRMS m/z calcd for _{C21H24BrClNO2} [M+H]⁺: 436.1; found 436.7. Purity of the compound was further confirmed by RP-HPLC by using method 1: Rₜ=8.54 min (98% pure; FIG. 12).

Synthesis of Compound 4.

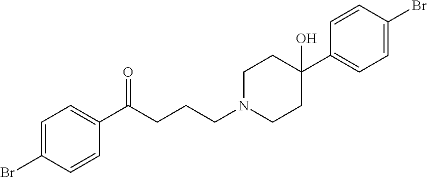 [SMILES: O=C(CCCN1CCC(O)(c2ccc(Br)cc2)CC1)c1ccc(Br)cc1]

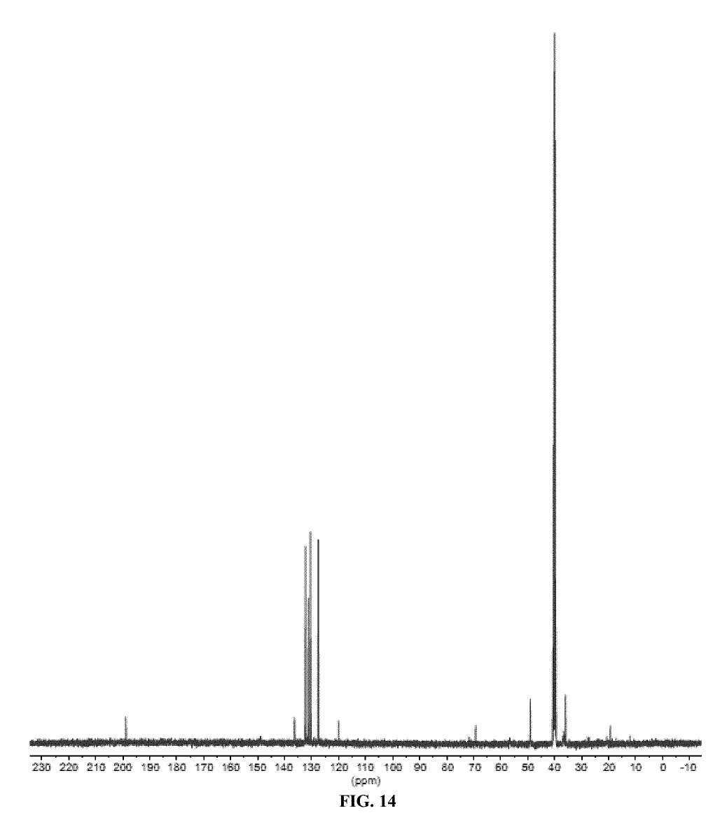

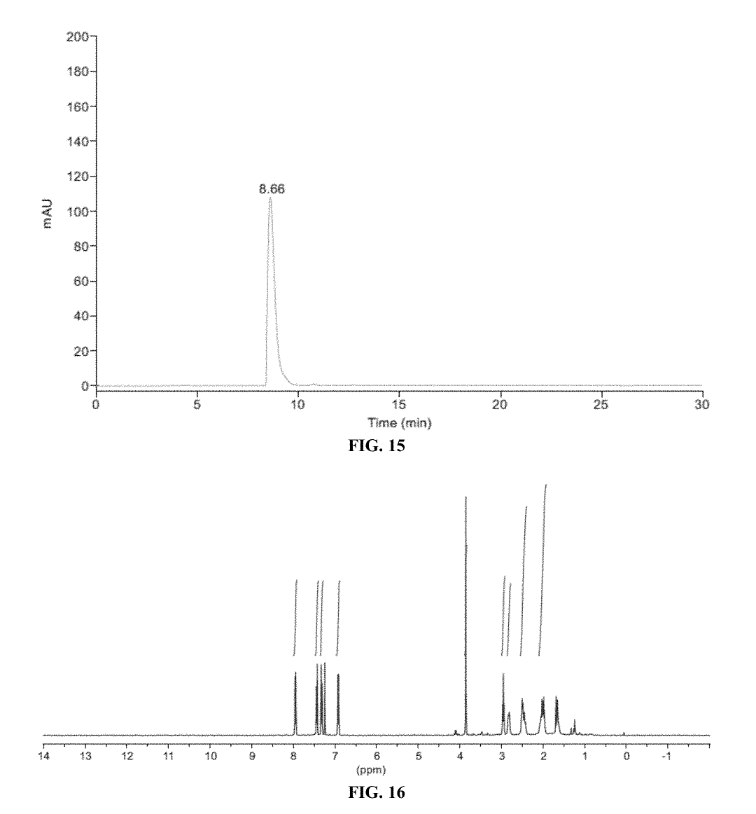

Following the general procedure described for the synthesis of compound 1, sodium iodide (0.039 g, 0.260 mmol), sodium carbonate (0.050 g, 0.472 mmol), 4′-bromo-4-chlorobutyrophenone (0.062 g, 0.236 mmol), and 4-(4-bromophenyl)-4-hydroxypiperidine (0.060 g, 0.236 mmol) in MeCN (2 mL) were used to afford compound 4 (0.034 g, 30%, R_{f}0.24 in MeOH:EtOAc/1:9) as a white solid: ¹H NMR (400 MHz, CDCl₃, FIG. 13) δ 7.81 (d, J=8.0 Hz, 2H), 7.59 (d, J=8.0 Hz, 2H), 7.43 (d, J=8.8 Hz, 2H), 7.33 (d, J=8.8 Hz, 2H), 3.14-3.07 (m, 2H), 3.06 (t, J=6.4 Hz, 2H), 2.96-2.80 (m, 2H), 2.79-2.69 (m, 2H), 2.48-2.24 (m, 2H), 2.12 (p, J=6.4 Hz, 2H), 1.78 (m, 2H); ¹³C NMR (100 MHz, (CD₃)₂SO, FIG. 14) δ 198.4, 135.9, 131.83, 131.77, 130.8, 130.0, 129.9, 127.1, 119.6, 68.8, 55.2, 48.5, 36.1, 35.4, 18.9; LRMS m/z calcd for _{C21H24Br2NO2} [M+H]⁺: 480.0; found 480.7. Purity of the compound was further confirmed by RP-HPLC by using method 1: Rₜ=8.66 min (99% pure; FIG. 15).

Synthesis of Compound 5.

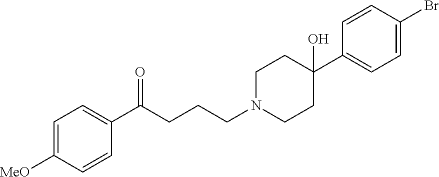 [SMILES: COc1ccc(C(=O)CCCN2CCC(O)(c3ccc(Br)cc3)CC2)cc1]

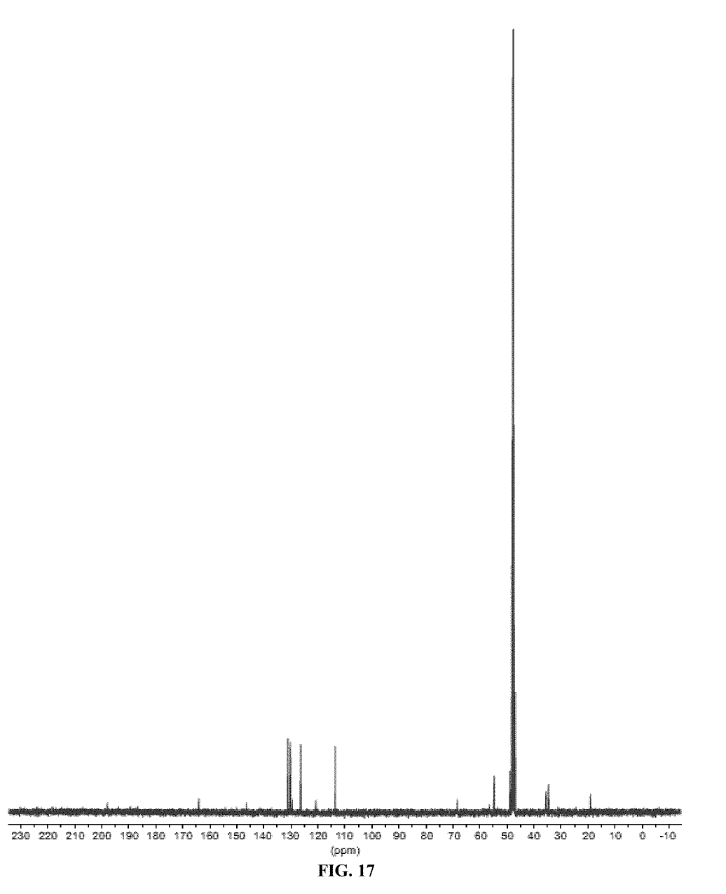

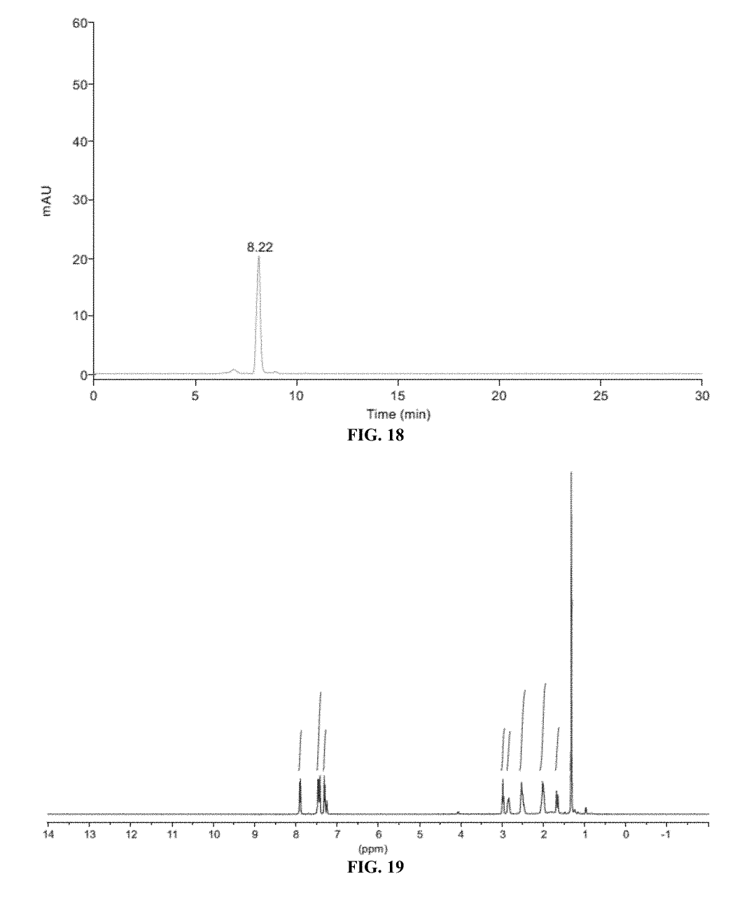

Following the general procedure described for the synthesis of compound 1, sodium iodide (0.039 g, 0.260 mmol), sodium carbonate (0.050 g, 0.472 mmol), 4′-methoxy-4-chlorobutyrophenone (0.050 g, 0.236 mmol), and 4-(4-bromophenyl)-4-hydroxypiperidine (0.060 g, 0.236 mmol) in MeCN (2 mL) were used to afford compound 5 (0.013 g, 13%, R_{f}0.20 in MeOH:EtOAc/1:9) as a white solid: ¹H NMR (400 MHz, CDCl₃, FIG. 16) δ 7.95 (d, J=8.4 Hz, 2H), 7.43 (d, J=8.4 Hz, 2H), 7.32 (d, J=8.4 Hz, 2H), 6.92 (d, J=8.4 Hz, 2H), 3.85 (s, 3H), 2.95 (t, J=6.8 Hz, 2H), 2.82 (m, 2H), 2.50 (t, J=6.8 Hz, 2H), 2.45 (t, J=11.6 Hz, 2H), 2.08-1.96 (m, 2H), 1.98 (p, J=6.8 Hz, 2H), 1.67 (m, 2H), 1.62 (br s, 1H); ₁₃C NMR (100 MHz, CD₃OD, FIG. 17) δ 199.5, 165.6, 148.1, 132.6, 131.7, 131.0, 128.0, 122.3, 115.1, 69.9, 56.2, 50.4, 49.6, 37.0, 36.1, 20.6; LRMS m/z calcd for C₂₂H₂₇BrNO₃[M+H]⁺: 432.1; found 432.7. Purity of the compound was further confirmed by RP-HPLC by using method 1: Rₜ=8.22 min (95% pure; FIG. 18).

2. Biochemistry and Microbiology

2.1. Biochemical/Biological Reagents and Instrumentation.

The azole antifungal agents fluconazole (FLC), itraconazole (ITC), ketoconazole (KCZ), posaconazole (POS), and voriconazole (VOR) were purchased from AK Scientific (Union city, CA, USA). The Candida albicans strains, including C. albicans ATCC 10231 (strain B), C. albicans ATCC 64124 (strain F), and C. albicans ATCC MYA-2876 (Strain E) were a generous gift from Dr. Jon Y. Takemoto (Utah State University, Logan, Utah, USA). The rest of the C. albicans strains, including C. albicans ATCC 90819 (strain G), C. albicans ATCC MYA-2310 (strain D), C. albicans ATCC MYA-1237 (strain C), and C. albicans ATCC MYA-1003 (strain A), as well as the non-albicans Candida fungus C. glabrata ATCC 2001 (strain H) were purchased from the American Type Culture Collection (ATCC, Manassas, Va., USA). The filamentous fungus Aspergillus terreus ATCC MYA-3633 (strain I) was also purchased from the ATCC. Yeast strains were cultured at 35° C. in yeast extract peptone dextrose (YEPD) broth. Filamentous fungi were cultured on potato dextrose agar (PDA, catalog #110130, EMD Millipore, Billerica, Mass., USA) at 25° C. before the spores were harvested. All fungal experiments were carried out in RPMI 1640 medium (catalog # R6504, Sigma-Aldrich, St. Louis, Mo., USA) buffered to pH 7.0 with 0.165 M MOPS buffer (Sigma-Aldrich, St. Louis, Mo., USA). Colorimetric assessments were performed by using a SpectraMax M5 spectrometer (Molecular Devices, Sunnyvale, Calif., USA).

2.2. Determination of Minimum Inhibitory Concentration (MIC) Values.

To assess the potential synergistic effect between various azoles and our bromperidol (2) as well as its derivatives 1 and 3-5, we first needed to determine the individual minimum inhibitory concentration (MIC) values of compounds 1-5 and of the azole antifungals against each fungal strain of interest to gauge the range of concentrations to use in further assays. These MIC values were determined using the broth microdilution method in sterile 96-well plates with the highest concentrations of drugs used being 128 μg/mL for compounds 2 and 5, 64 μg/mL for compounds 1 and 4, and 32 μg/mL for compound 3 and for the azole antifungals. Note: The different starting concentrations result from the solubility limit of the compounds studied. These compounds were serially diluted (two-fold dilutions) horizontally on the plate in 100 μL of RPMI medium. A diluted yeast culture (25 μL of a fungal stock with _{OD600} of 0.125 in 10 mL of RPMI medium, which achieves a final inoculum size of around 1-5×10³ CFU/mL) was plated across the plate (100 μL per well), making a final volume of 200 μL total per well. Similarly, in vitro MIC values for compounds 1-5 against filamentous fungi were determined as previously described in CLSI document M38-A2.³ Briefly, 1×10⁵ spores were seeded in each well in 100 μL of RPMI medium. The MIC value of each compound was observed by visual inspection after 48 h of incubation at 35° C. for yeast or 72 h at 35° C. for the Aspergillus strains (TABLES 2 and 4).

2.3. Combination Studies of Azoles and Bromperidol Series Derivatives by Checkerboard Assays.

To assess the potential synergistic effect between the commercially available azoles and our compounds 1-5 against various fungal strains, we employed the standard checkerboard assay as previously described with slight variations. The commercially available azoles were serially diluted (two-fold dilutions) in the 96-well plates while the second drug, compounds 1-5, were double-diluted in tubes outside of the 96-well plates and then later added into the plates using a multi-channel pipet. The concentration of azoles varied horizontally while that of compounds 1-5 varied vertically. The appropriate range of concentrations for each compound was determined based on their corresponding MIC values against each fungal strain. The inoculum size for yeast and filamentous fungi were the same as in the MIC experiments described in section 2.2. The 96-well plates were incubated at 35° C. for 48 h for yeasts and 72 h for the Aspergillus strain before visual inspection for growth. The observed MIC values of the azoles and compounds 1-5 alone as well as the MIC values for the two compounds in combo were then used to calculate the fractional inhibitory concentration index (FICI) using the formula below. The combinational effect of the two tested compounds were considered synergistic (abbreviated SYN) if FICI<0.5, additive (abbreviated ADD) if 0.5<FICI<4, and antagonistic if FICI>4 (TABLES 2-4).

$$F I C I = \frac{MIC of {azole}_{combo}}{MIC of {azole}_{alone}} + \frac{MIC of our {compound}_{combo}}{MIC of our {compound}_{alone}}$$

2.4. Time-Kill Assay.

In order to observe the time-dependent killing effect of the combination of azoles and bromperidol (2) and its derivatives 1 and 3-5, we selected two representative combinations, compound 2 with either POS or VOR, and tested the effect on C. albicans ATCC 64124 (strain F) as previously described with modifications. An overnight culture of C. albicans ATCC 64124 (Strain F) in YEPD broth was inoculated at 1×10⁵ CFU/mL density in liquid RPMI 1640 medium at 35° C. for each sample, including growth control, azole (POS or VOR) alone, compound 2 alone, and the combination of azole (POS or VOR) and compound 2 at 0.5×MIC, 1×MIC, 4×MIC, and 8×MIC concentrations (for the combination of VOR and compound 2, only 1×MIC and 8×MIC combinations were performed). The concentrations of azoles or compound 2 used in the azole or compound 2 alone samples were equal to the highest concentration of that drug used in the combination samples (e.g., the concentration of POS in the 8×MIC combination sample is 32 μg/mL, therefore, the concentration of POS in POS alone sample is 32 μg/mL). Each sample was incubated at 35° C. with shaking at 200 rpm. At various time points (0, 3, 6, 9, 12, and 24 h), 100 μL aliquots were taken from each sample and serially diluted (10-fold dilutions) in sterile ddH₂O. 100 μL of each dilution was spread onto PDA plates and incubated at 35° C. for 48 h before colony counts were determined (FIGS. 2A-B). At the end of the 24-h experiment, 50 μL of 1 mM sterile resazurin solution was added to each sample for visual comparison of growth. Each experiment was performed in duplicate.

2.5. Biofilm Disruption Assay.

In order to investigate the synergistic effect of azoles and our compounds on inhibiting biofilm formation of C. albicans ATCC 64124 (strain F), we performed a biofilm disruption assay in a checkerboard setup and used XTT [2,3-bis(2-methoxy-4-nitro-5-sulfophenyl)-2H-tetrazolium-5-carboxanilide] reduction to examine the metabolic activity of the biofilm. Briefly, C. albicans ATCC 64124 (strain F) was grown overnight in YEPD broth at 35° C. The culture was then diluted to an OD₆₀₀ of 0.12 (equivalent to 1×10⁶ CFU/mL) and 100 μL was placed into each well of a 96-well plate. The cells were incubated at 37° C. for 24 h for growth. The following day, the spent medium containing planktonic cells was aspirated and each wells were carefully washed three times with sterile phosphate buffer saline (PBS). The washed biofilm was supplemented with drug-containing RPMI 1640 medium with POS or VOR concentrations varying vertically and the concentration of compound 2 varied horizontally in a manner similar to that described above in the checkerboard assay section. The negative control (medium only) and the positive control (biofilm plus medium) wells were also included in the same 96-well plate. The plates were then incubated at 37° C. statically for additional 24 h and then washed with sterile PBS. 100 μL of a XTT (0.5 mg/mL)/menadione (1 μM) solution was added to each well, covered with aluminum foil, and incubated for 2 h at 37° C. Then, 80 μL of the colored supernatant from each well was transferred to a new 96-well plate, and the absorption was read at OD₄₉₀. The percent metabolic activity of the formed biofilm at various drug concentration combinations was calculated by dividing the metabolic activity of biofilm formed for that well by that of the biofilm formed in the growth control well (in the absence of any drug). For these experiments, the sessile MIC (SMIC₉₉) was determined, which is defined as the drug concentration required to inhibit the metabolic activity of biofilm by 99% compared to the growth control. The SMIC₉₉ values were used to calculate the FICI as described above and summarized in TABLE 5. The assay for each combination was performed in duplicates.

2.6. Mammalian Cytotoxicity Assays.

In order to evaluate the potential cytotoxic effect of the azoles and compounds 1-5 combinations against mammalian cells, we performed mammalian cytotoxicity assays as previously described with minor modifications. The human embryonic kidney cell line HEK-293 was purchased from ATCC (Manassas, Va.), whereas the human bronchial epithelial cells BEAS-2B and the murine macrophage cells J774A.1 were generous gifts from Prof. David K. Orren (University of Kentucky, Lexington, Ky.) and Prof. David J. Feola (University of Kentucky, Lexington, Ky.), respectively. HEK-293 and BEAS-2B cells were cultured in Dulbecco's Modified Eagle's Medium (DMEM, catalog #11965-092, Thermo Fisher Scientific, Waltham, Mass.) supplemented with 10% fetal bovine serum (FBS, ATCC, Manassas, Va.) and 1% penicillin/streptomycin (ATCC, Manassas, Va.) at 37° C. with 5% CO₂. J774A.1 was cultured in a different DMEM (catalog #30-2002, ATCC, Manassas, Va.) with the same supplements in the medium at 37° C. with 5% CO₂. HEK-293 and BEAS-2B cells were dislodged by treating with a solution comprised of 0.05% trypsin and 0.53 mM EDTA (ATCC, Manassas, Va.) when passaging, whereas J774A.1 cells were dislodged from the culture flask by mechanical scraping.

All cytotoxicity assays were performed in quadruplicates in 96-well plates where both HEK-293 and J774A.1 cells were seeded at 1×10⁴ cells per well and BEAS-2B cells were seeded at 3×10³ cells per well. For the assessment of the toxicity of POS, VOR, and compound 2 alone as well as in combination, mammalian cytotoxicity assay were performed as previously described. The concentration of azoles tested in the assay ranged from 0.06 to 16 μg/mL, whereas that of compound 2 ranged from 0.25 to 64 μg/mL. When testing combination toxicity between azoles and compound 2, the combination of various concentrations of azoles was first tested in the presence of 8 μg/mL of compound 2 for all three cell lines due to the higher toxicity that compound 2 exerted on J774A.1 cells. Combination toxicity was additionally tested between either azoles and compound 2 in the presence of 32 μg/mL of compound 2 for BEAS-2B and HEK-293 cells in order to gain a better understanding of the toxicity for these two cells at higher concentrations of compound 2. Since many xenobiotics stimulate cell growth instead of exerting toxicity at sub-IC₅₀ concentrations, resulting in >100% cell survival in the treatment groups, these >100% cell survival data was considered as no observed toxicity and expressed them as 100% cell survival (FIGS. 3A-D).

### Example 3

Synthesis of Compound 6.

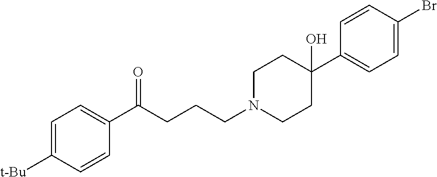 [SMILES: CC(C)(C)c1ccc(C(=O)CCCN2CCC(O)(c3ccc(Br)cc3)CC2)cc1]

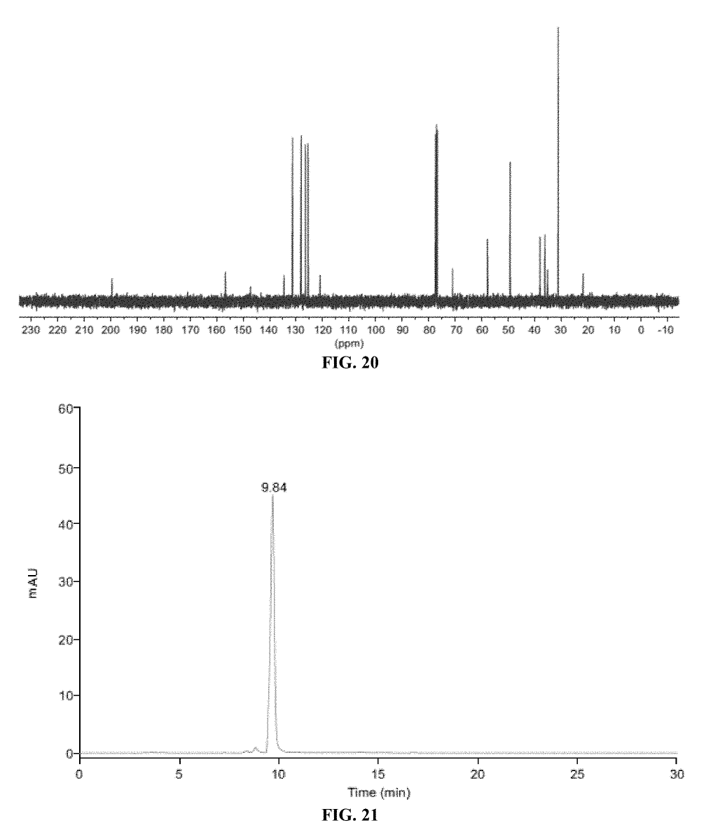

Following the general procedure described for the synthesis of compound 1, sodium iodide (0.039 g, 0.260 mmol), sodium carbonate (0.050 g, 0.472 mmol), 4′-tert-butyl-4-chlorobutyrophenone (0.056 g, 0.236 mmol), and 4-(4-bromophenyl)-4-hydroxypiperidine (0.060 g, 0.236 mmol) in MeCN (2 mL) were used to afford compound 6 (0.030 g, 28%, Rf 0.30 in MeOH:EtOAc/1:9) as a white solid: ¹H NMR (400 MHz, CDCl₃, FIG. 19) δ 7.90 (d, J=8.4 Hz, 2H), 7.45 (d, J=8.0 Hz, 2H), 7.42 (d, J=8.4 Hz, 2H), 7.30 (d, J=8.0 Hz, 2H), 2.98 (t, J=6.8 Hz, 2H), 2.84 (m, 2H), 2.54-2.45 (m, 4H), 2.06-1.96 (m, 2H), 2.00 (p, J=6.8 Hz, 2H), 1.81 (very br s, 1H), 1.66 (m, 2H), 1.32 (s, 9H); ¹³C NMR (100 MHz, CDCl₃, FIG. 20) δ 199.5, 156.7, 147.2, 134.5, 131.3, 128.0, 126.4, 125.5, 120.9, 70.9, 57.8, 49.2, 37.9, 36.1, 35.1, 31.1, 21.6; LRMS m/z calcd for _{C25H33BrNO2} [M+H]⁺: 458.2; found 458.8. Purity of the compound was further confirmed by RP-HPLC by using method 1: Rt=9.84 min (98% pure; FIG. 21).

General Procedure for the Amination Reaction (e.g., Synthesis of Compound 7).

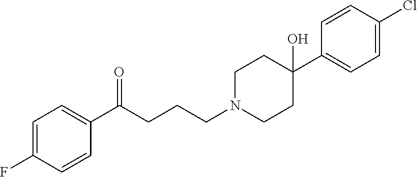 [SMILES: O=C(CCCN1CCC(O)(c2ccc(Cl)cc2)CC1)c1ccc(F)cc1]

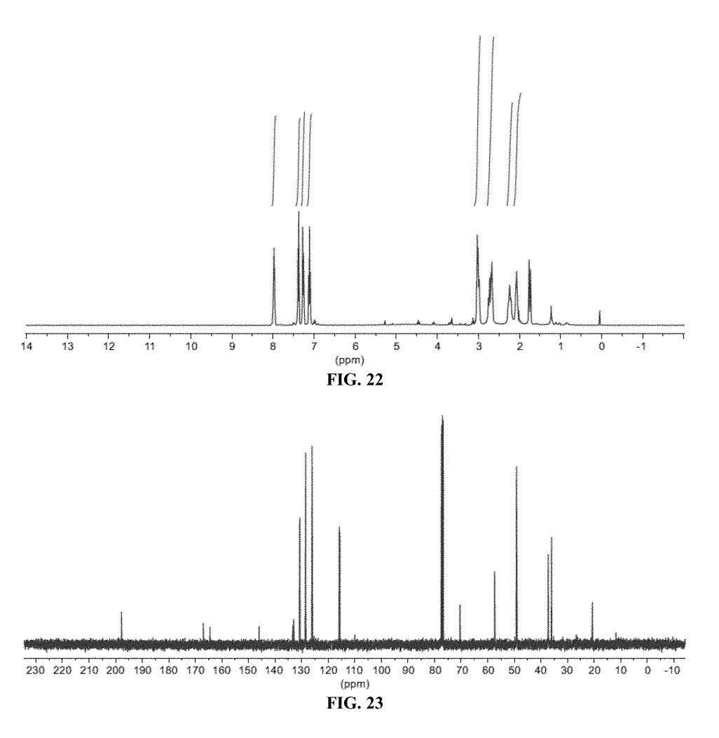

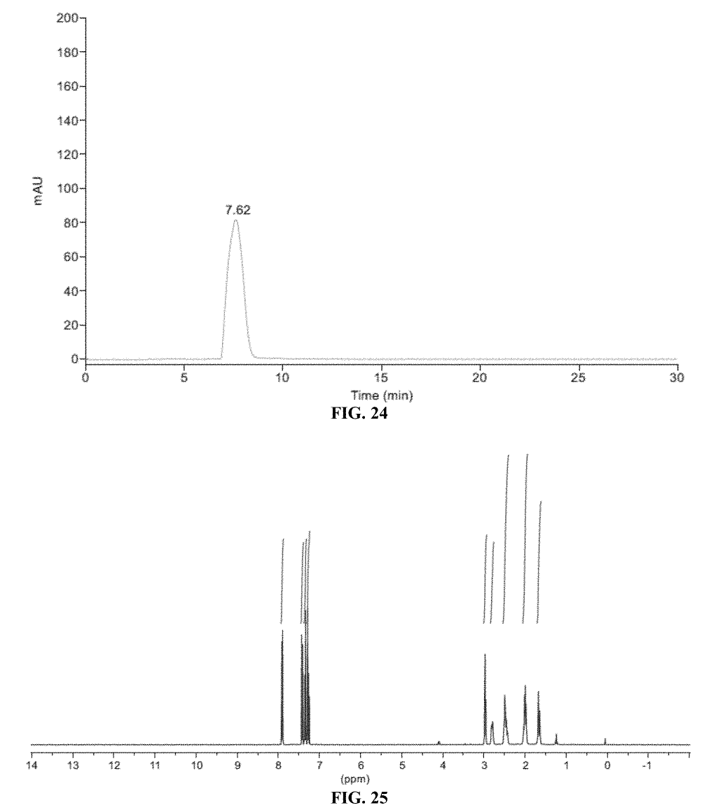

Compound 7 was prepared following a previously published protocol for a similar molecule.¹ Sodium iodide (0.039 g, 0.260 mmol) and sodium carbonate (0.050 g, 0.472 mmol) were added to a stirred mixture of 4′-fluoro-4-chlorobutyrophenone (0.047 g, 0.236 mmol) and 4-(4-chlorophenyl)-4-hydroxypiperidine (0.050 g, 0.236 mmol) in MeCN (2 mL). The reaction mixture was refluxed for 12 h. The mixture was diluted with H₂O, and extracted with CH₂Cl₂ (3×10 mL), dried over MgSO₄, and concentrated under reduced pressure. The residue was purified by column chromatography (SiO₂, MeOH:EtOAc/1:9, R_{f} 0.19), to give compound 7 (0.027 g, 30% yield) as a white solid: ¹H NMR (400 MHz, CDCl₃, FIG. 22) δ 7.99-7.96 (m, 2H), 7.38 (d, J=7.6 Hz, 2H), 7.27 (dd, J=8.4, 1.6 Hz, 2H), 7.11 (app. t, J=8.4 Hz, 2H), 3.04-2.98 (m, 4H), 2.72 (t, J=11.6 Hz, 2H), 2.67 (t, J=6.8 Hz, 2H), 2.30-2.17 (m, 2H), 2.07 (p, J=6.8 Hz, 2H), 1.75 (m, 2H); ¹³C NMR (100 MHz, CDCl₃, FIG. 23) δ 197.8, 167.0, 164.5, 146.0, 133.2, 133.0, 130.7, 130.6, 128.4, 126.0, 115.8, 115.6, 70.4, 57.4, 49.1, 37.2, 35.9, 20.6; LRMS m/z calcd for C₂₁H₂₄ClFNO₂ [M+H]⁺: 376.1; found 376.7. Purity of the compound was further confirmed by RP-HPLC by using method 1: Rt=7.62 min (100% pure; FIG. 24).

Synthesis of Compound 8.

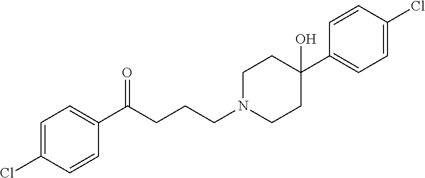 [SMILES: O=C(CCCN1CCC(O)(c2ccc(Cl)cc2)CC1)c1ccc(Cl)cc1]

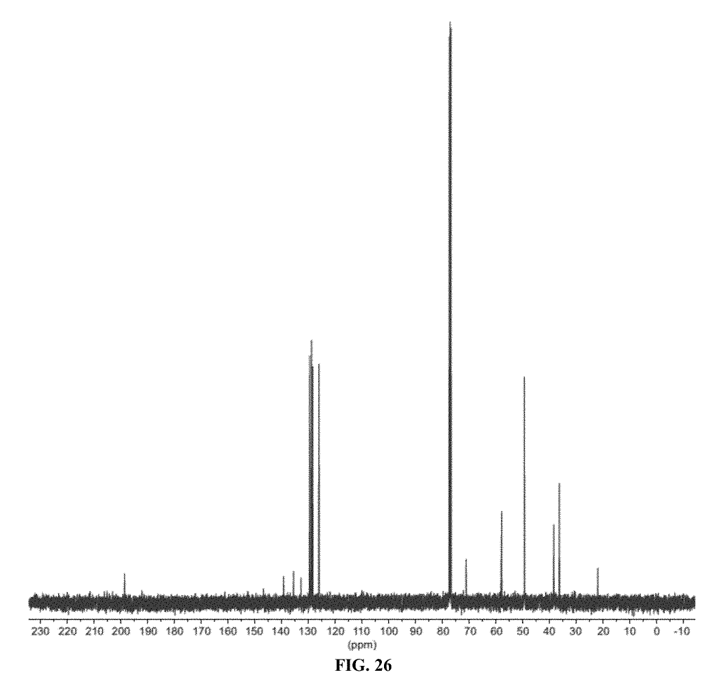

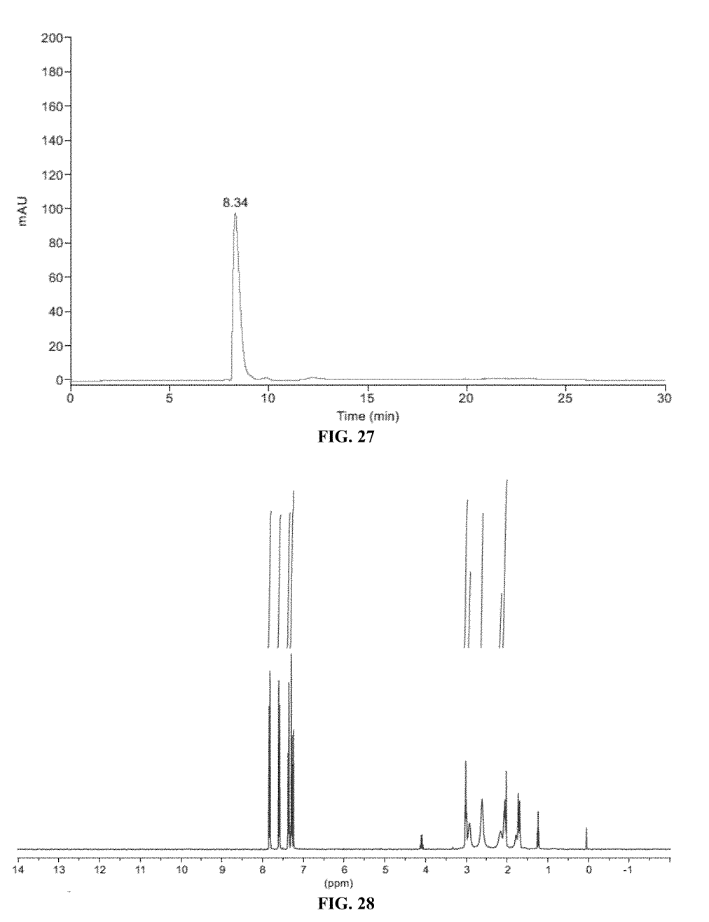

Following the general procedure described for the synthesis of compound 7, sodium iodide (0.11 g, 0.70 mmol), sodium carbonate (0.14 g, 1.26 mmol), 4′-chloro-4-chlorobutyrophenone (0.14 g, 0.63 mmol), and 4-(4-chlorophenyl)-4-hydroxypiperidine (0.14 g, 0.63 mmol) in MeCN (6 mL) were used to afford compound 8 (0.038 g, 15%, Rf 0.18 in MeOH:EtOAc/5:95) as a white solid: ¹H NMR (400 MHz, CDCl₃, FIG. 25) δ 7.90 (dt, J=8.4, 2.0 Hz, 2H), 7.42 (dt, J=8.8, 2.0 Hz, 2H), 7.34 (dt, J=8.8, 2.0 Hz, 2H), 7.27 (dt, J=8.4, 2.0 Hz, 2H), 2.97 (t, J=6.8 Hz, 2H), 2.80 (m, 2H), 2.51-2.42 (m, 4H), 2.04-1.96 (m, 2H), 1.99 (p, J=6.8 Hz, 2H) 1.67-1.64 (m, 3H); ¹³C NMR (100 MHz, CDCl₃, FIG. 26) δ 198.6, 146.8, 139.3, 135.5, 132.7, 129.5, 128.8, 128.3, 126.0, 71.0, 57.7, 49.2, 38.2, 36.2, 21.7; LRMS m/z calcd for C21H24Cl2NO2 [M+H]⁺: 392.1; found 392.7. Purity of the compound was further confirmed by RP-HPLC by using method 1: Rt=8.34 min (97% pure; FIG. 27).

Synthesis of Compound 9.

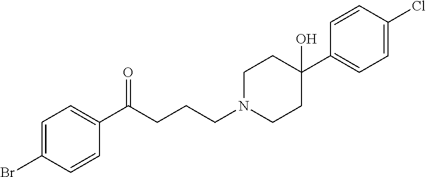 [SMILES: O=C(CCCN1CCC(O)(c2ccc(Cl)cc2)CC1)c1ccc(Br)cc1]

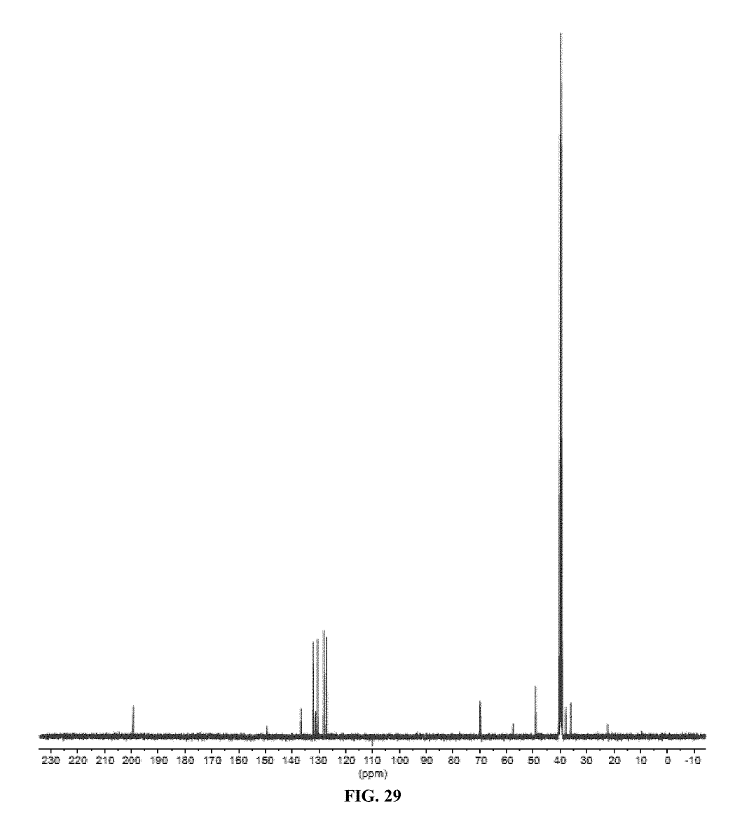

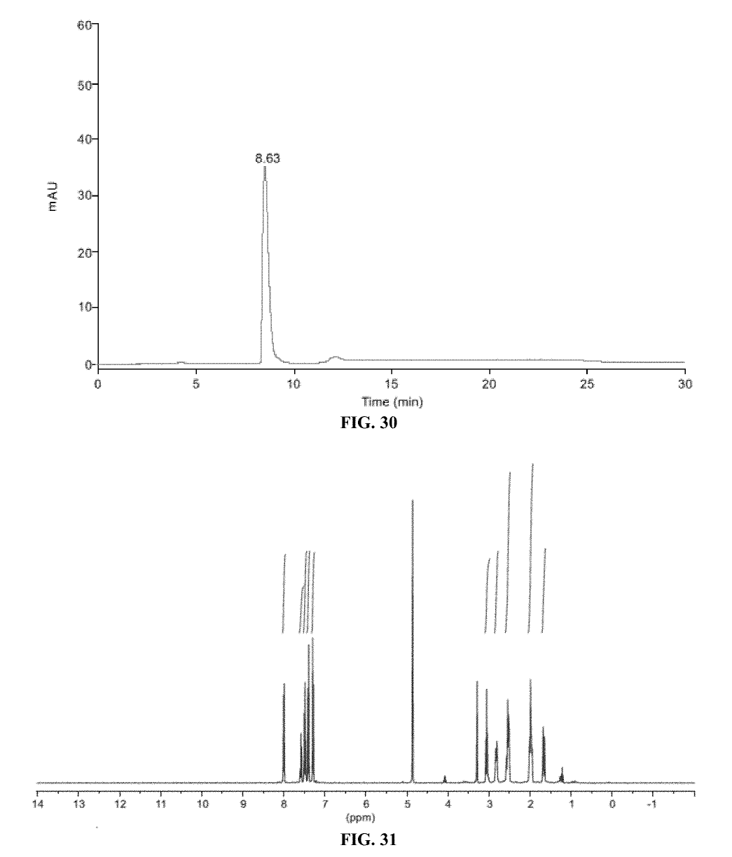

Following the general procedure described for the synthesis of compound 7, sodium iodide (0.11 g, 0.70 mmol), sodium carbonate (0.14 g, 1.26 mmol), 4′-bromo-4-chlorobutyrophenone (0.17 g, 0.63 mmol), and 4-(4-chlorophenyl)-4-hydroxypiperidine (0.14 g, 0.63 mmol) in MeCN (6 mL) were used to afford compound 9 (0.042 g, 12%, Rf 0.18 in MeOH:EtOAc/5:95) as a white solid: ¹H NMR (400 MHz, CDCl₃, FIG. 28) δ 7.83 (d, J=8.4 Hz, 2H), 7.59 (d, J=8.4 Hz, 2H), 7.36 (d, J=8.4 Hz, 2H), 7.28 (d, J=8.4 Hz, 2H), 3.01 (t, J=6.8 Hz, 2H), 2.92 (m, 2H), 2.70-2.54 (m, 4H), 2.24-2.10 (m, 2H), 2.05 (p, J=6.8 Hz, 2H), 1.78 (br s, 1H), 1.70 (m, 2H); ¹³C NMR (100 MHz, (CD₃)₂SO, FIG. 29) δ 199.0, 149.1, 136.3, 131.8, 130.9, 130.2, 127.8, 127.0, 126.8, 69.5, 57.2, 48.9, 37.5, 35.7, 21.9; LRMS m/z calcd for C21H24ClBrNO2 [M+H]⁺: 436.1; found 436.7. Purity of the compound was further confirmed by RP-HPLC by using method 1: Rt=8.63 min (98% pure; FIG. 30).

Synthesis of Compound 10.

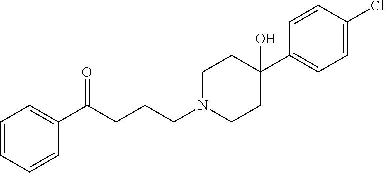 [SMILES: O=C(CCCN1CCC(O)(c2ccc(Cl)cc2)CC1)c1ccccc1]

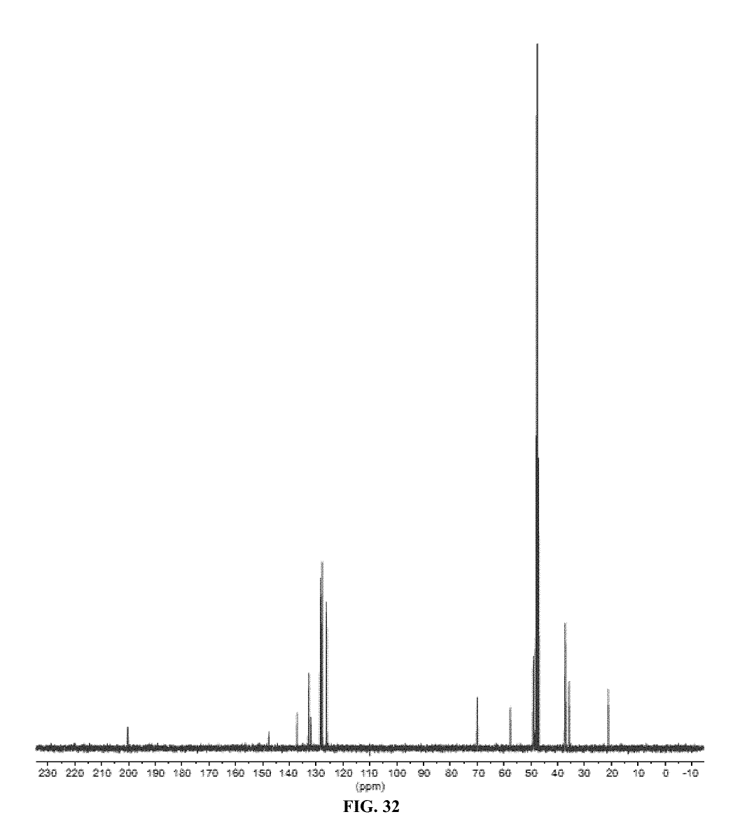

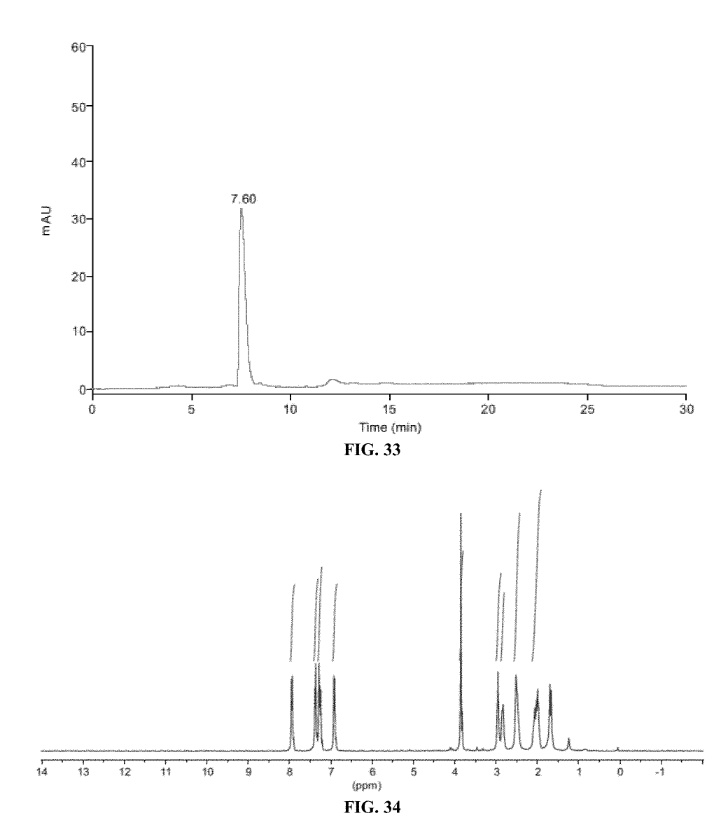

Following the general procedure described for the synthesis of compound 7, sodium iodide (0.039 g, 0.260 mmol), sodium carbonate (0.050 g, 0.472 mmol), 4-chlorobutyrophenone (0.043 g, 0.236 mmol), and 4-(4-chlorophenyl)-4-hydroxypiperidine (0.050 g, 0.236 mmol) in MeCN (2 mL) were used to afford compound 10 (0.014 g, 17%, Rf 0.12 in MeOH:EtOAc/5:95) as a light yellow solid: ¹H NMR (400 MHz, CD₃OD, FIG. 31) δ 8.02 (d, J=8.8 Hz, 2H), 7.60 (t, J=6.8 Hz, 1H), 7.50 (t, J=6.8 Hz, 2H), 7.42 (d, J=8.8 Hz, 2H), 7.31 (d, J=8.4 Hz, 2H), 3.08 (t, J=6.8 Hz, 2H), 2.84 (m, 2H), 2.59-2.53 (m, 4H), 2.04-1.97 (m, 4H), 1.69 (m, 2H); ¹³C NMR (100 MHz, CD₃OD, FIG. 32) δ 201.9, 149.2, 138.6, 134.3, 133.6, 129.9, 129.33, 129.29, 127.7, 71.5, 59.2, 50.5, 38.7, 37.3, 22.7; LRMS m/z calcd for C21H25ClNO2 [M+H]⁺: 358.2; found 358.8. Purity of the compound was further confirmed by RP-HPLC by using method 1: Rt=7.60 min (96% pure; FIG. 33).

Synthesis of Compound 11.

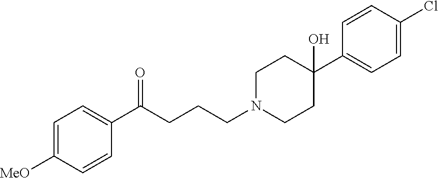 [SMILES: COc1ccc(C(=O)CCCN2CCC(O)(c3ccc(Cl)cc3)CC2)cc1]

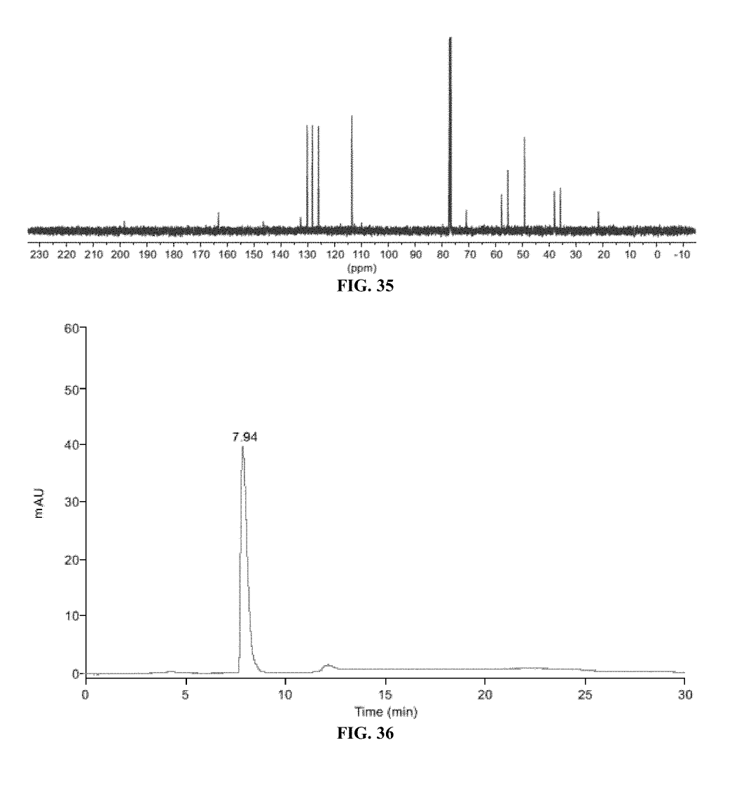

Following the general procedure described for the synthesis of compound 7, sodium iodide (0.21 g, 1.39 mmol), sodium carbonate (0.27 g, 2.52 mmol), 4′-methoxy-4-chlorobutyrophenone (0.27 g, 1.26 mmol), and 4-(4-chlorophenyl)-4-hydroxypiperidine (0.27 g, 1.26 mmol) in MeCN (11 mL) were used to afford compound 11 (0.041 g, 8%, Rf 0.14 in MeOH:EtOAc/1:9) as a white solid: ¹H NMR (400 MHz, CDCl₃, FIG. 34) δ 7.94 (d, J=8.4 Hz, 2H), 7.37 (d, J=8.4 Hz, 2H), 7.27 (d, J=8.4 Hz, 2H), 6.91 (d, J=8.4 Hz, 2H), 3.85 (s, 3H), 2.95 (t, J=6.8 Hz, 2H), 2.83 (m, 2H), 2.58-2.42 (m, 4H), 2.10-1.96 (m, 2H), 1.98 (p, J=6.8 Hz, 2H), 1.70-1.66 (m, 3H); ¹³C NMR (100 MHz, CDCl₃, FIG. 35) δ 198.5, 163.4, 146.7, 132.8, 130.3, 130.2, 128.4, 126.1, 113.7, 71.0, 57.9, 55.5, 49.3, 38.1, 35.9, 21.7; LRMS m/z calcd for C22H27ClNO3 [M+H]⁺: 388.2; found 388.7. Purity of the compound was further confirmed by RP-HPLC by using method 1: Rt=7.94 min (98% pure; FIG. 36).

Synthesis of Compound 12.

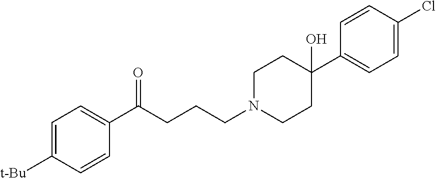 [SMILES: CC(C)(C)c1ccc(C(=O)CCCN2CCC(O)(c3ccc(Cl)cc3)CC2)cc1]

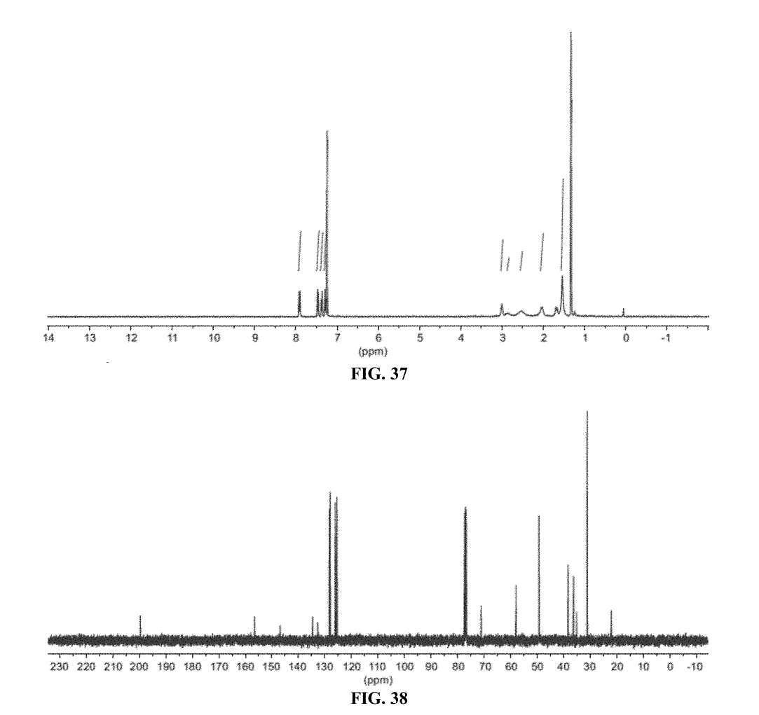

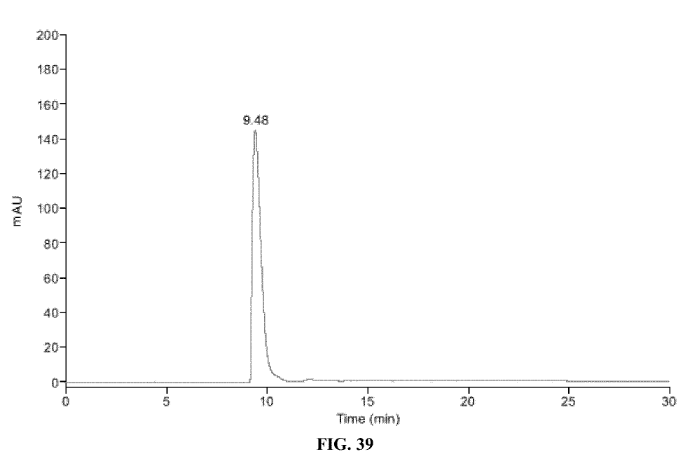

Following the general procedure described for the synthesis of compound 7, sodium iodide (0.21 g, 1.39 mmol), sodium carbonate (0.27 g, 2.52 mmol), 4′-tert-butyl-4-chlorobutyrophenone (0.30 g, 1.26 mmol), and 4-(4-chlorophenyl)-4-hydroxypiperidine (0.27 g, 1.26 mmol) in MeCN (11 mL) were used to afford compound 12 (0.12 g, 23%, Rf 0.14 in MeOH:EtOAc/5:95) as a white solid: ¹H NMR (400 MHz, CDCl₃, FIG. 37) δ 7.90 (d, J=8.4 Hz, 2H), 7.45 (d, J=8.4 Hz, 2H), 7.36 (d, J=8.4 Hz, 2H), 7.28 (d, J=8.4 Hz, 2H), 2.97 (t, J=6.8 Hz, 2H), 2.79 (m, 2H), 2.49-2.38 (m, 4H), 2.03-1.95 (m, 4H), 1.66-1.62 (m, 3H), 1.32 (s, 9H); ¹³C NMR (100 MHz, CDCl₃, FIG. 38) δ 199.6, 156.6, 146.9, 134.6, 132.6, 128.3, 128.0, 126.1, 125.4, 71.1, 57.9, 49.3, 38.3, 36.2, 35.1, 31.1, 22.0; LRMS m/z calcd for C25H33ClNO2 [M+H]⁺: 414.2; found 414.7. Purity of the compound was further confirmed by RP-HPLC by using method 1: Rt=9.48 min (100% pure; FIG. 39).

Synthesis of Compound 13.

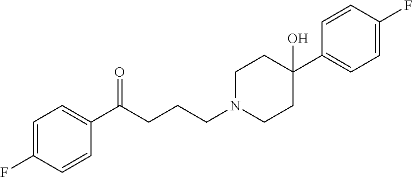 [SMILES: O=C(CCCN1CCC(O)(c2ccc(F)cc2)CC1)c1ccc(F)cc1]

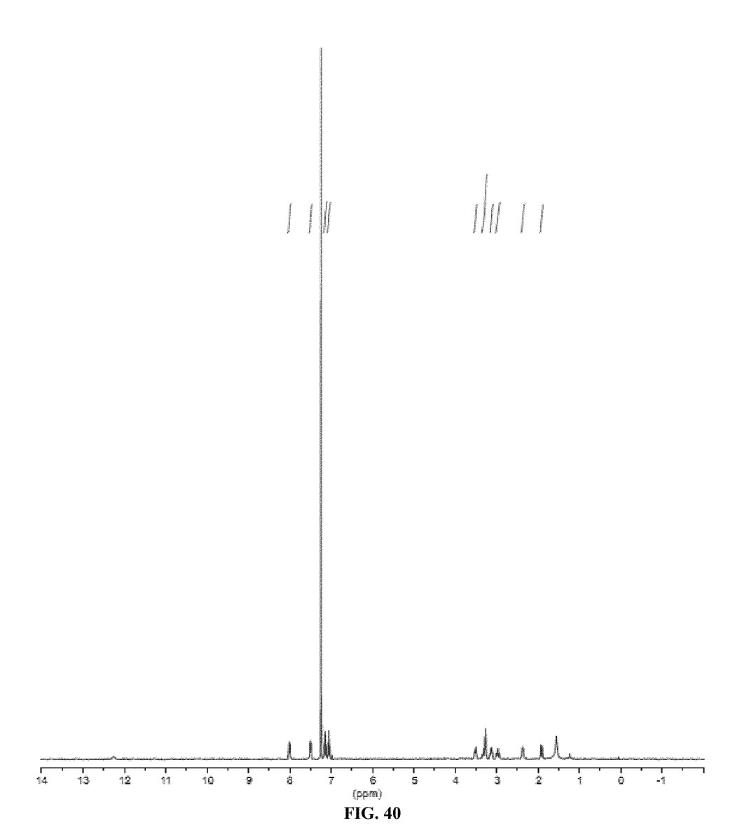

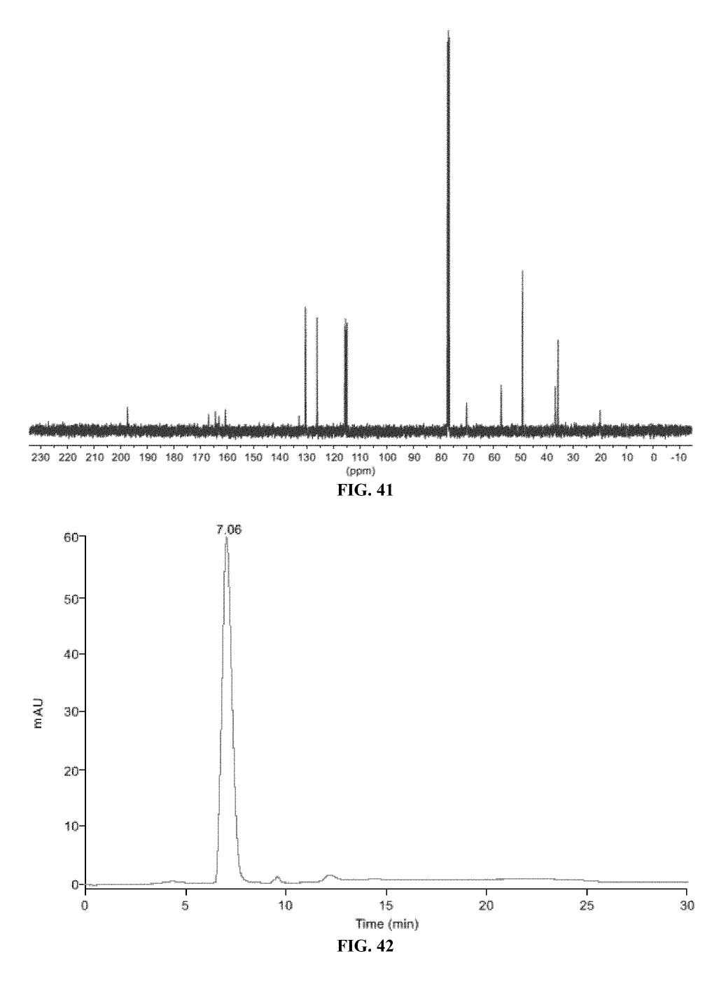

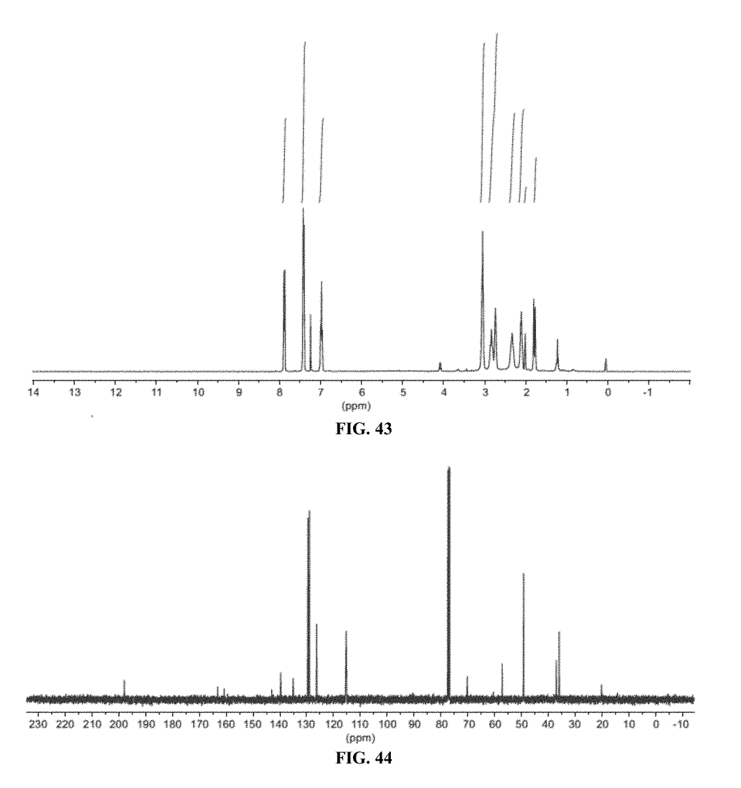

Following the general procedure described for the synthesis of compound 7, sodium iodide (0.039 g, 0.260 mmol), sodium carbonate (0.050 g, 0.472 mmol), 4′-fluoro-4-chlorobutyrophenone (0.047 g, 0.236 mmol), and 4-(4-fluorophenyl)-4-hydroxypiperidine (0.046 g, 0.236 mmol) in MeCN (2 mL) were used to afford compound 13 (0.011 g, 13%, Rf 0.14 in MeOH:EtOAc/1:9) as a white solid: ¹H NMR (400 MHz, CDCl₃, FIG. 40) δ 8.01 (dd, J=8.4, 5.6 Hz, 2H), 7.50 (dd, J=8.4, 5.6 Hz, 2H), 7.14 (app. t, J=8.4 Hz, 2H), 7.05 (app. t, J=8.4 Hz, 2H), 3.52-3.47 (m, 2H), 3.31 (t, J=10.0 Hz, 2H), 3.26 (t, J=6.8 Hz, 2H), 3.15-3.10 (m, 2H), 2.97 (td, J=12.8, 4.0 Hz, 2H), 2.36 (p, J=6.8 Hz, 2H), 1.91 (m, 2H); ¹³C NMR (100 MHz, CDCl₃, FIG. 41) δ 197.5, 167.1, 164.5, 163.2, 160.7, 133.1, 130.7, 130.6, 126.3, 126.2, 115.9, 115.7, 115.3, 115.1, 70.1, 57.2, 49.1, 36.9, 35.8, 20.0; LRMS m/z calcd for C21H24F2NO2 [M+H]⁺: 360.2; found 360.7. Purity of the compound was further confirmed by RP-HPLC by using method 1: Rt=7.06 min (98% pure; FIG. 42).

Synthesis of Compound 14.

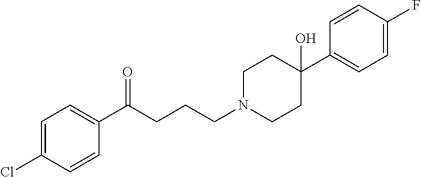 [SMILES: O=C(CCCN1CCC(O)(c2ccc(F)cc2)CC1)c1ccc(Cl)cc1]

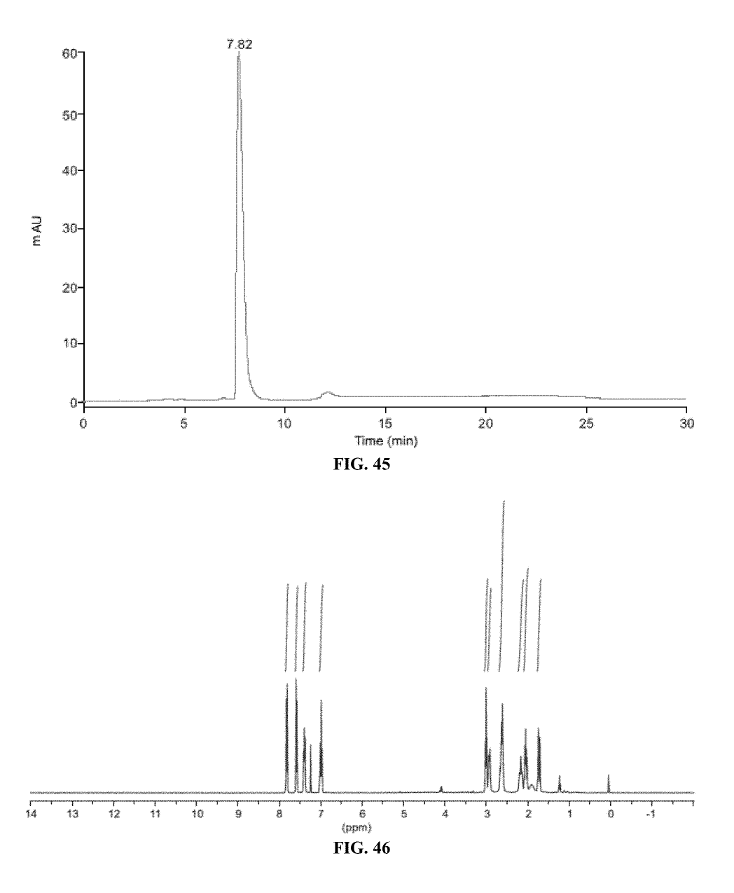

Following the general procedure described for the synthesis of compound 7, sodium iodide (0.039 g, 0.260 mmol), sodium carbonate (0.050 g, 0.472 mmol), 4′-chloro-4-chlorobutyrophenone (0.051 g, 0.236 mmol), and 4-(4-fluorophenyl)-4-hydroxypiperidine (0.046 g, 0.236 mmol) in MeCN (2 mL) were used to afford compound 14 (0.018 g, 20%, Rf 0.24 in MeOH:EtOAc/1:9) as a white solid: ¹H NMR (400 MHz, CDCl₃, FIG. 43) δ 7.88 (d, J=8.4 Hz, 2H), 7.42-7.40 (m, 4H), 6.97 (app. t, J=8.4 Hz, 2H), 3.05 (t, J=6.8 Hz, 4H), 2.83 (t, J=10.8 Hz, 2H), 2.73 (m, 2H), 2.33 (m, 2H), 2.10 (p, J=6.8 Hz, 2H), 1.79 (m, 2H); ¹³C NMR (100 MHz, CDCl₃, FIG. 44) δ 198.0, 163.1, 160.7, 143.0, 139.6, 135.0, 129.5, 128.9, 126.3, 126.2, 115.2, 115.0, 70.1, 57.1, 49.1, 36.9, 35.9, 20.1; LRMS m/z calcd for _{C21H24ClFNO2} [M+H]⁺: 376.1; found 376.7. Purity of the compound was further confirmed by RP-HPLC by using method 1: Rt=7.82 min (98% pure; FIG. 45).

Synthesis of Compound 15.

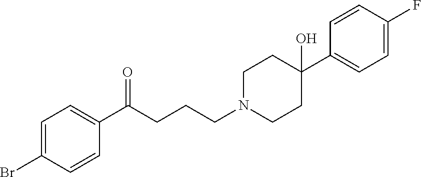 [SMILES: O=C(CCCN1CCC(O)(c2ccc(F)cc2)CC1)c1ccc(Br)cc1]

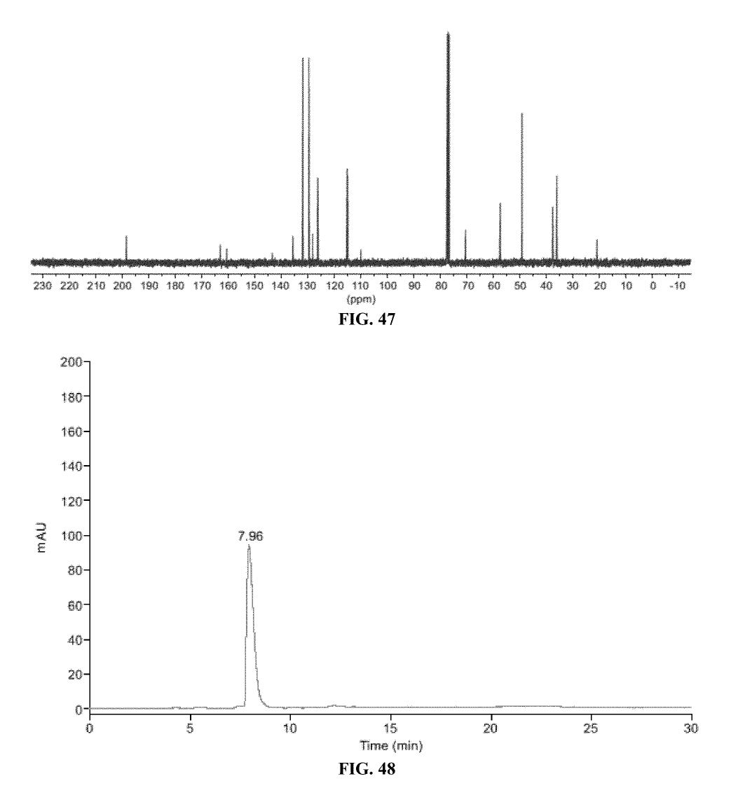

Following the general procedure described for the synthesis of compound 7, sodium iodide (0.039 g, 0.260 mmol), sodium carbonate (0.050 g, 0.472 mmol), 4′-bromo-4-chlorobutyrophenone (0.062 g, 0.236 mmol), and 4-(4-fluorophenyl)-4-hydroxypiperidine (0.046 g, 0.236 mmol) in MeCN (2 mL) were used to afford compound 15 (0.016 g, 16%, Rf 0.21 in MeOH:EtOAc/1:9) as a white solid: ¹H NMR (400 MHz, CDCl₃, FIG. 46) δ 7.82 (d, J=8.4 Hz, 2H), 7.58 (d, J=8.4 Hz, 2H), 7.39 (dd, J=8.4, 5.6 Hz, 2H), 6.99 (app. t, J=8.4 Hz, 2H), 3.00 (t, J=6.8 Hz, 2H), 2.92 (m, 2H), 2.67-2.59 (m, 4H), 2.17 (t, J=11.2 Hz, 2H), 2.05 (p, J=6.8 Hz, 2H), 1.90 (br s, 1H), 1.73 (m, 2H); ¹³C NMR (100 MHz, CDCl₃, FIG. 47) δ 198.5, 163.1, 160.6, 143.4, 135.7, 131.9, 129.6, 128.2, 126.3, 126.2, 115.2, 115.0, 70.5, 57.4, 49.2, 37.6, 36.0, 20.9; LRMS m/z calcd for _{C21H24FBrNO2} [M+H]⁺: 420.1; found 420.7. Purity of the compound was further confirmed by RP-HPLC by using method 1: Rt=7.96 min (99% pure; FIG. 48).

Synthesis of Compound 16.

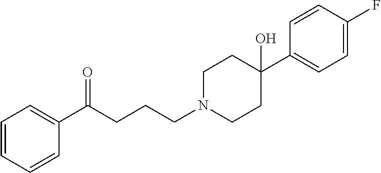 [SMILES: O=C(CCCN1CCC(O)(c2ccc(F)cc2)CC1)c1ccccc1]

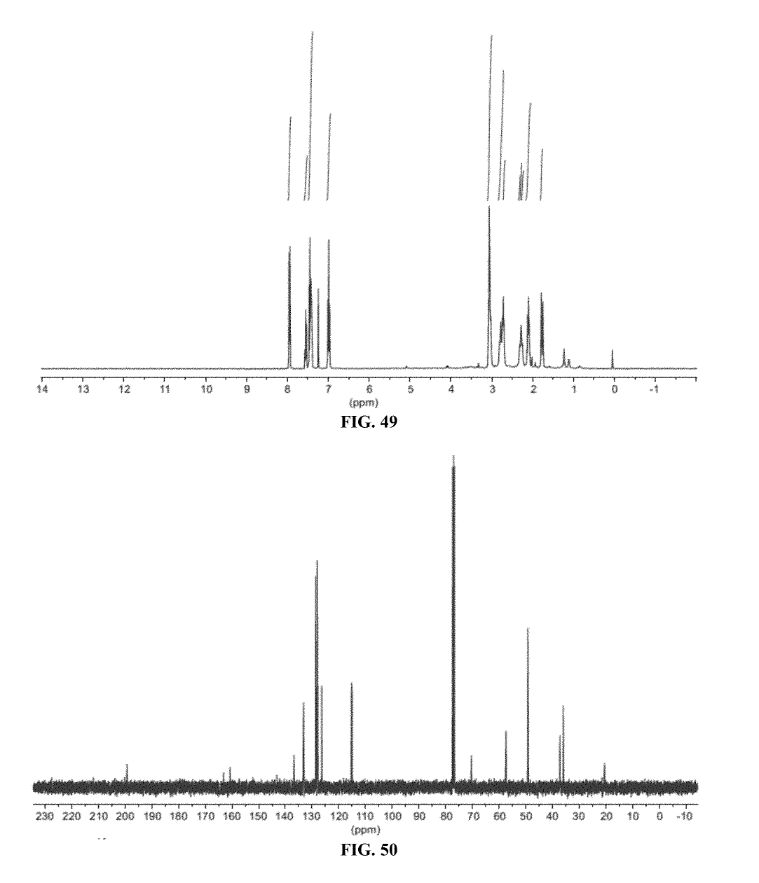

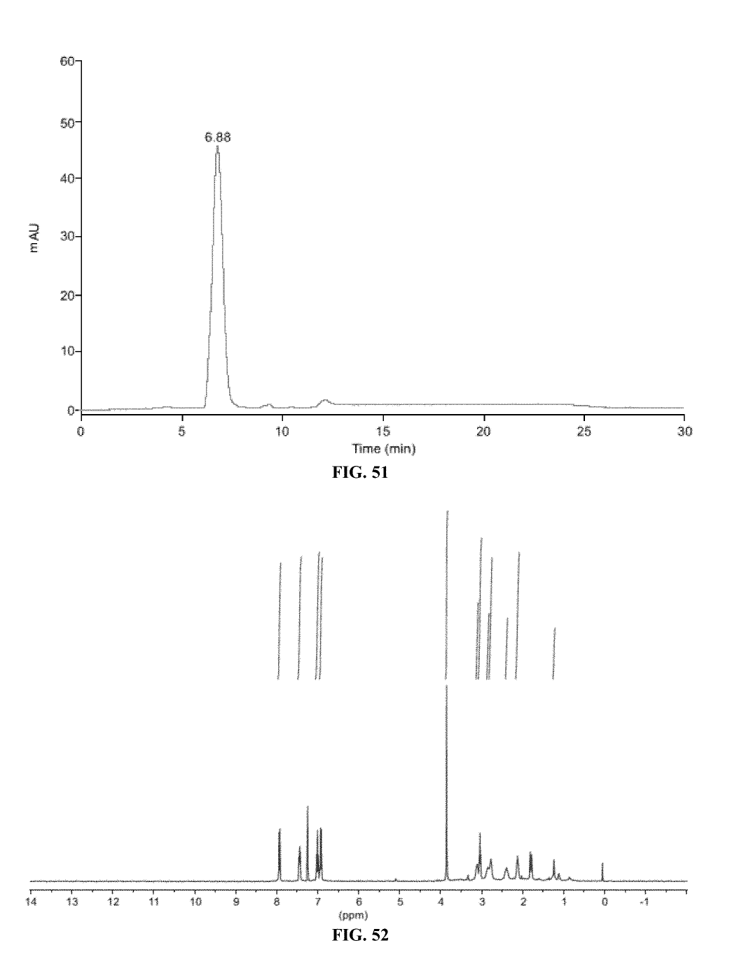

Following the general procedure described for the synthesis of compound 7, sodium iodide (0.039 g, 0.260 mmol), sodium carbonate (0.050 g, 0.472 mmol), 4-chlorobutyrophenone (0.043 g, 0.236 mmol), and 4-(4-fluorophenyl)-4-hydroxypiperidine (0.046 g, 0.236 mmol) in MeCN (2 mL) were used to afford compound 16 (0.012 g, 15%, Rf 0.23 in MeOH:EtOAc/1:9) as a white solid: ¹H NMR (400 MHz, CDCl₃, FIG. 49) δ 7.95 (d, J=7.6 Hz, 2H), 7.55 (t, J=7.6, 2.0 Hz, 1H), 7.45 (d, J=7.2 Hz, 2H), 7.43-7.40 (m, 2H), 6.98 (app. t, J=7.2 Hz, 2H), 3.06 (t, J=6.8 Hz, 2H), 3.05-3.02 (m, 2H), 2.78 (t, J=10.8 Hz, 2H), 2.72 (t, J=7.2 Hz, 2H), 2.28 (t, J=10.8 Hz, 2H), 2.10 (p, J=6.8 Hz, 2H), 1.77 (m, 2H); ¹³C NMR (100 MHz, CDCl₃, FIG. 50) δ 199.3, 163.1, 160.7, 143.1, 136.8, 133.2, 128.6, 128.0, 126.3, 126.2, 115.2, 115.0, 70.3, 57.3, 49.1, 37.2, 35.9, 20.4; LRMS m/z calcd for _{C21H25FNO2} [M+H]⁺: 342.2; found 342.8. Purity of the compound was further confirmed by RP-HPLC by using method 1: Rt=6.88 min (98% pure; FIG. 51).

Synthesis of Compound 17.

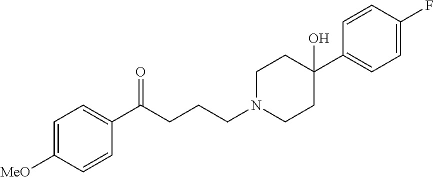 [SMILES: COc1ccc(C(=O)CCCN2CCC(O)(c3ccc(F)cc3)CC2)cc1]

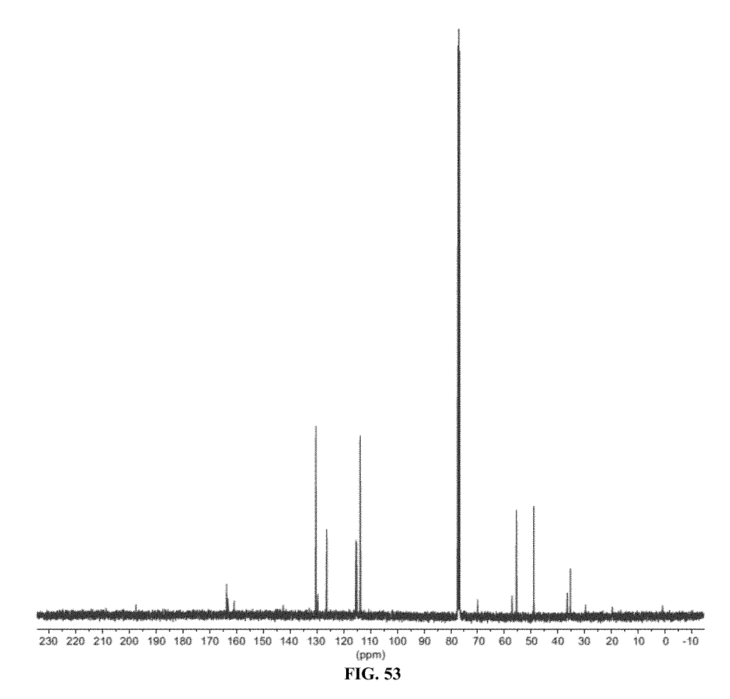

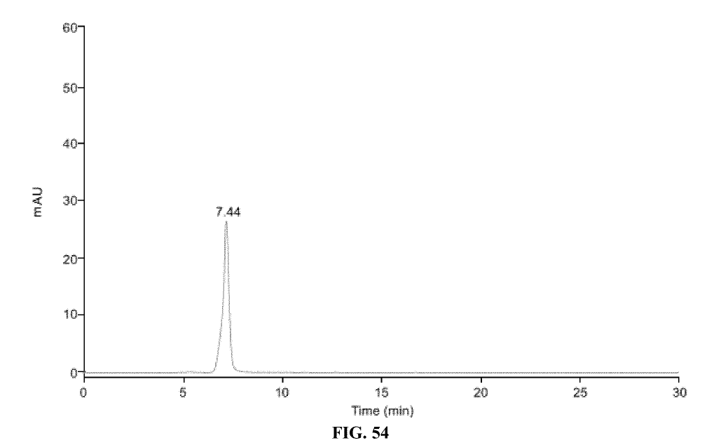

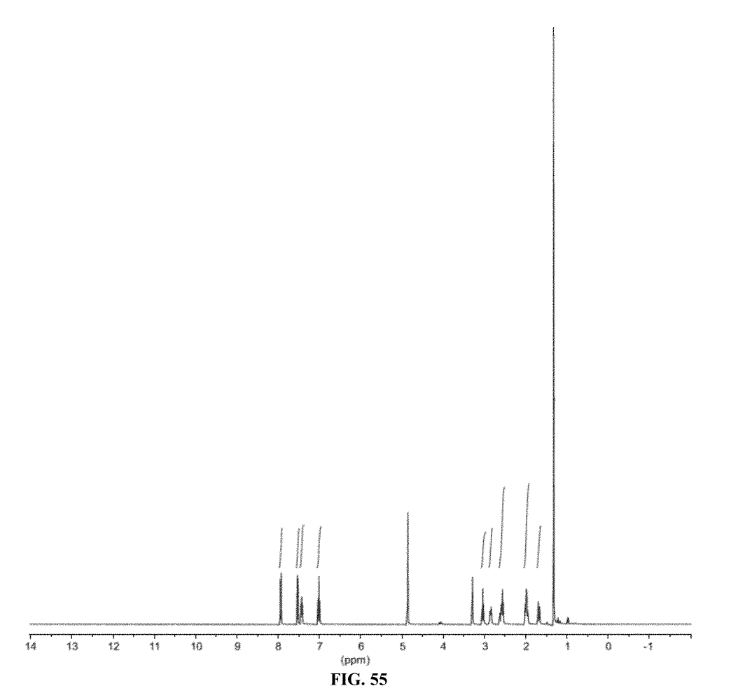

Following the general procedure described for the synthesis of compound 7, sodium iodide (0.039 g, 0.260 mmol), sodium carbonate (0.050 g, 0.472 mmol), 4′-methoxy-4-chlorobutyrophenone (0.050 g, 0.236 mmol), and 4-(4-fluorophenyl)-4-hydroxypiperidine (0.046 g, 0.236 mmol) in MeCN (2 mL) were used to afford compound 17 (0.006 g, 7%, Rf 0.19 in MeOH:EtOAc/1:9) as a white solid: ¹H NMR (400 MHz, CDCl₃, FIG. 52) δ 7.93 (d, J=8.8 Hz, 2H), 7.44 (dd, J=8.0, 2.0 Hz, 2H), 7.00 (app. t, J=8.0 Hz, 2H), 6.92 (d, J=8.8 Hz, 2H), 3.85 (s, 3H), 3.12-3.06 (m, 2H), 3.04 (t, J=6.8 Hz, 2H), 2.90-2.72 (m, 4H), 2.44-2.33 (m, 2H), 2.12 (p, J=6.8 Hz, 2H), 1.79 (m, 2H); ¹³C NMR (100 MHz, CDCl₃, FIG. 53) δ 197.5, 163.6, 163.2, 160.8, 142.5, 130.3, 129.6, 126.34, 126.26, 115.4, 115.1, 113.8, 70.0, 57.1, 55.5, 49.0, 36.5, 35.3, 19.7; LRMS m/z calcd for _{C22H27FNO3} [M+H]⁺: 372.2; found 372.7. Purity of the compound was further confirmed by RP-HPLC by using method 1: Rt=7.44 min (100% pure; FIG. 54).

Synthesis of Compound 18.

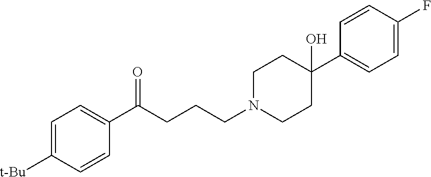 [SMILES: CC(C)(C)c1ccc(C(=O)CCCN2CCC(O)(c3ccc(F)cc3)CC2)cc1]

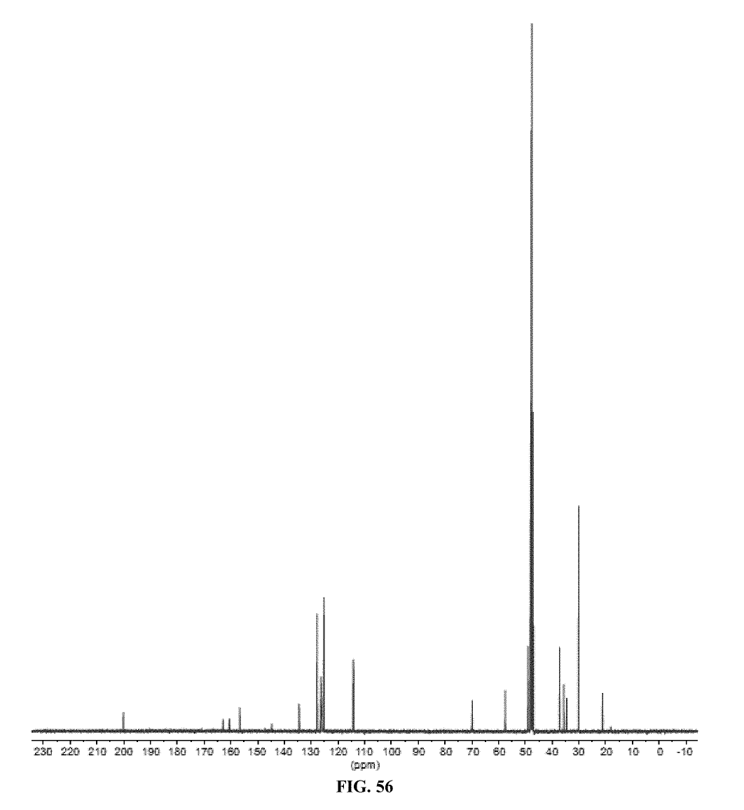

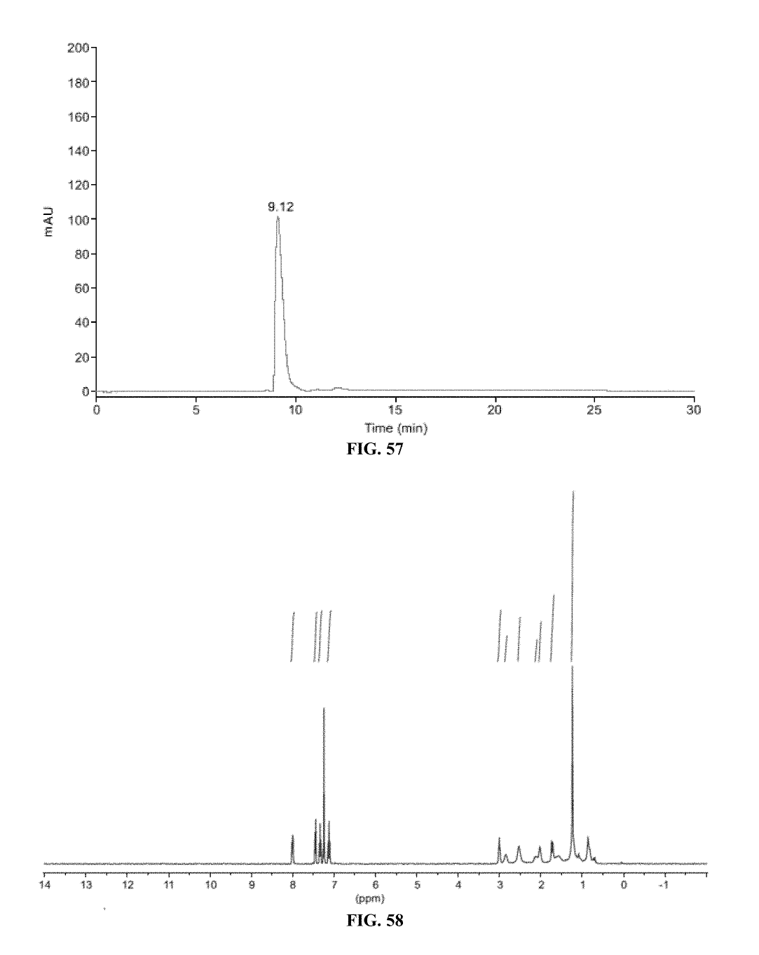

Following the general procedure described for the synthesis of compound 7, sodium iodide (0.039 g, 0.260 mmol), sodium carbonate (0.050 g, 0.472 mmol), 4′-tert-butyl-4-chlorobutyrophenone (0.056 g, 0.236 mmol), and 4-(4-fluorophenyl)-4-hydroxypiperidine (0.046 g, 0.236 mmol) in MeCN (2 mL) were used to afford compound 18 (0.022 g, 23%, Rf 0.11 in MeOH:EtOAc/5:95) as a white solid: ¹H NMR (400 MHz, CD₃OD, FIG. 55) δ 7.95 (d, J=8.4 Hz, 2H), 7.55 (d, J=8.4 Hz, 2H), 7.45 (dd, J=8.4, 5.6 Hz, 2H), 7.03 (app. t, J=8.4 Hz, 2H), 3.06 (t, J=6.4 Hz, 2H), 2.87 (m, 2H), 2.64-2.56 (m, 4H), 2.04-1.96 (m, 4H), 1.70 (m, 2H), 1.35 (s, 9H); ¹³C NMR (100 MHz, CD₃OD, FIG. 56) δ 201.6, 164.5, 162.0, 158.2, 146.3, 136.0, 129.3, 127.9, 127.8, 126.8, 115.9, 115.7, 71.3, 59.1, 50.6, 38.7, 37.2, 36.1, 31.6, 22.7; LRMS m/z calcd for _{C25H33FNO2} [M+H]⁺: 398.2; found 398.8. Purity of the compound was further confirmed by RP-HPLC by using method 1: Rt=9.12 min (98% pure; FIG. 57).

Synthesis of Compound 19.

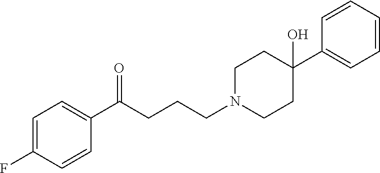 [SMILES: O=C(CCCN1CCC(O)(c2ccccc2)CC1)c1ccc(F)cc1]

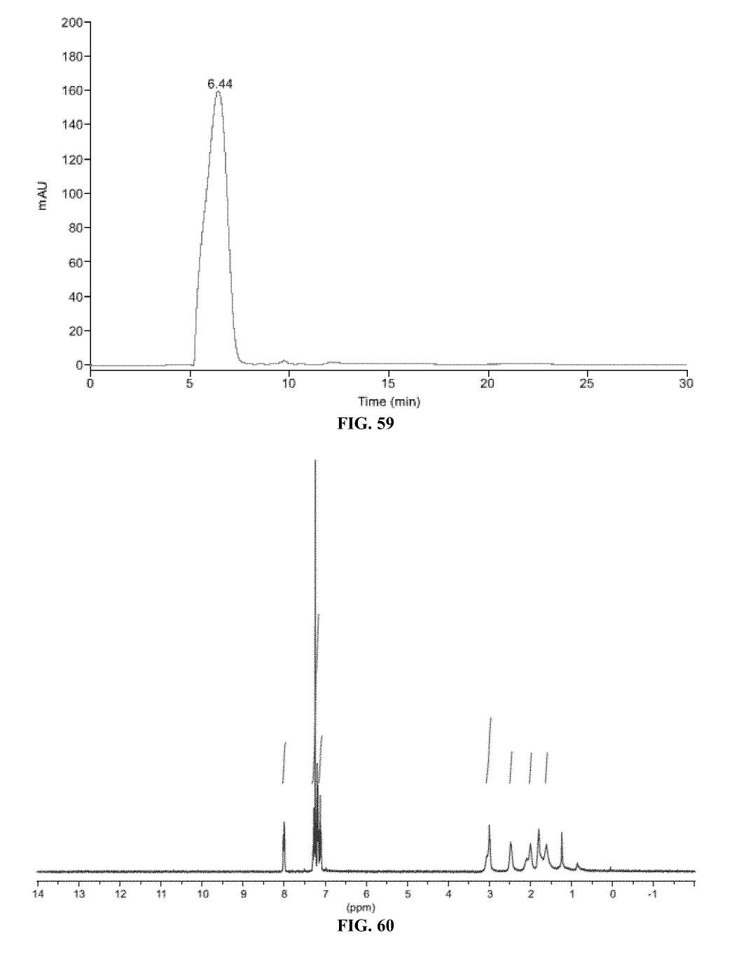

Following the general procedure described for the synthesis of compound 7, KIO3 (0.19 g, 0.90 mmol), potassium carbonate (0.24 g, 1.74 mmol), 4′-fluoro-4-chlorobutyrophenone (0.19 g, 0.92 mmol), and 4-phenylpiperidin-4-ol (0.10 g, 0.56 mmol) in toluene (10 mL) were used to afford compound 19 (0.051 g, 27%, Rf 0.20 in MeOH:CH2Cl2/5:95) as a yellow solid: ¹H NMR (400 MHz, CD₃OD, which matches lit², FIG. 58) δ 8.09 (m, 2H), 7.46 (d, J=7.6 Hz, 2H), 7.32 (t, J=7.6 Hz, 2H), 7.25-7.20 (m, 3H), 2.89-2.86 (m, 2H), 2.62 (t, J=11.6 Hz, 2H), 2.57 (t, J=8.0 Hz, 2H), 2.11-1.98 (m, 4H), 1.75-1.72 (m, 2H), 1.36-1.24 (m, 2H), 1.29 (very br s, 1H). Purity of the compound was further confirmed by RP-HPLC by using method 1: Rt=6.44 min (100% pure; FIG. 59).

Synthesis of Compound 20.

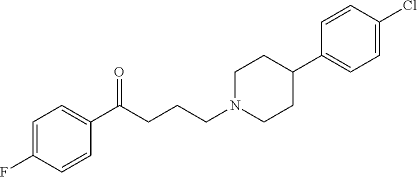 [SMILES: O=C(CCCN1CCC(c2ccc(Cl)cc2)CC1)c1ccc(F)cc1]

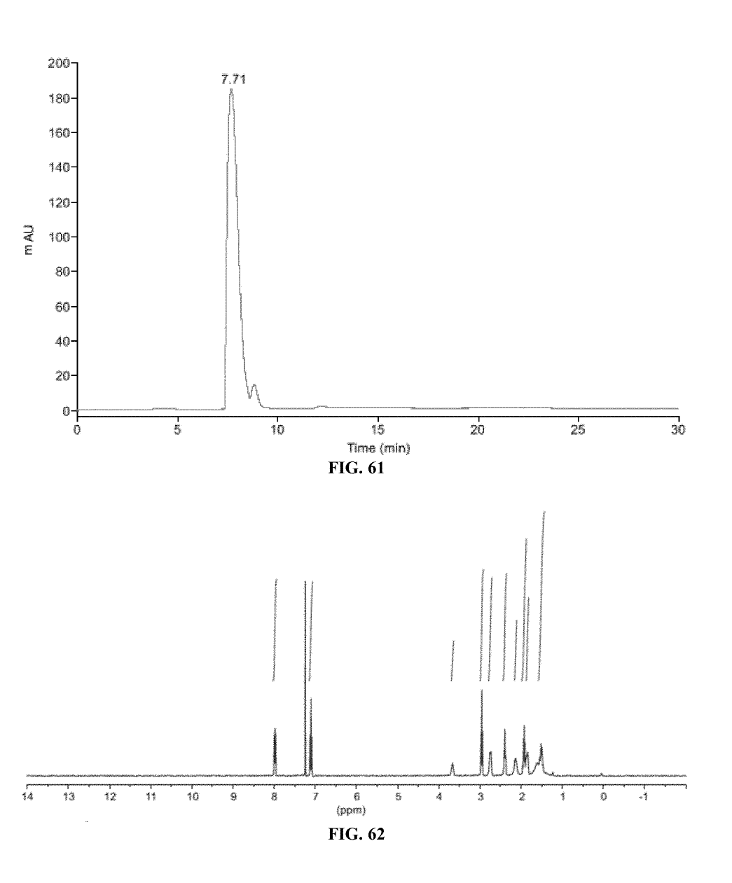

Compound 27 (0.07 g, 0.20 mmol) and Pd (20 mg, 10% on activated charcoal) were added to deoxygenated absolute EtOH (10 mL). The hydrogen was applied via a balloon. The reaction mixture was stirred at room temperature for 2 h, and then filtered through Celite®. The filtrate was collected and the solvent was evaporated under reduced pressure to yield compound 20 (0.035 g, 50% yield) as a white solid: ¹H NMR (400 MHz, CDCl₃, which matches lit.³, FIG. 60) δ 8.00 (dd, J=8.4, 6.0 Hz, 2H), 7.30-7.24 (app. t, J=8.0 Hz, 2H), 7.18 (d, J=7.2 Hz, 2H), 7.12 (app. t, J=8.4 Hz, 2H), 3.14-3.00 (m, 2H), 3.00 (t, J=6.8 Hz, 2H), 2.54-2.42 (m, 2H), 2.14-2.03 (m, 1H), 2.04-1.94 (m, 2H), 1.84-1.76 (m, 3H), 1.66-1.52 (m, 3H). Purity of the compound was further confirmed by RP-HPLC by using method 1: Rt=7.71 min (96% pure; FIG. 61).

Synthesis of Compound 21.

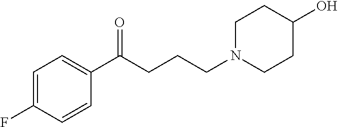 [SMILES: O=C(CCCN1CCC(O)CC1)c1ccc(F)cc1]

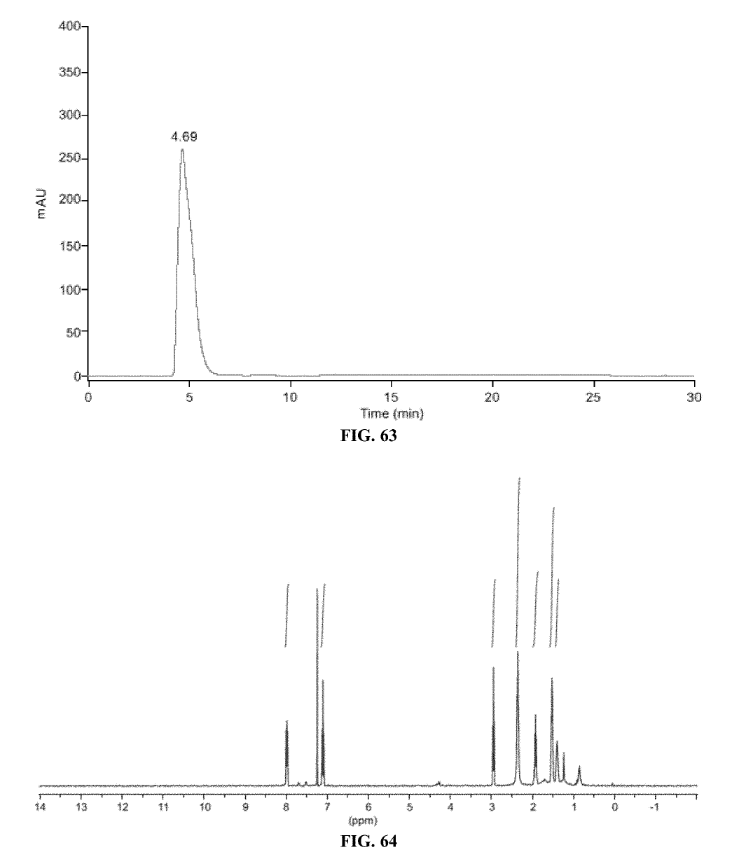

Following the general procedure described for the synthesis of compound 7, potassium iodide (0.65 g, 3.94 mmol), potassium carbonate (0.82 g, 5.91 mmol), 4′-fluoro-4-chlorobutyrophenone (0.59 g, 2.94 mmol), and 4-hydroxypiperidine (0.20 g, 1.97 mmol) in toluene (10 mL) were used to afford the known compound 21 (0.072 g, 14%, Rf 0.20 in MeOH:CH2Cl2/1:9) as a white solid: ¹H NMR (400 MHz, CDCl₃, which matches lit.⁴, FIG. 62) δ 7.98 (dd, J=8.8, 5.6 Hz, 2H), 7.06 (app. t, J=8.8 Hz, 2H), 3.65-3.61 (m, 1H), 2.95 (t, J=7.2 Hz, 2H), 2.76-2.73 (m, 2H), 2.39 (t, J=7.2 Hz, 2H), 2.19-2.08 (m, 2H), 1.92 (p, J=7.2 Hz, 2H), 1.89-1.84 (m, 2H), 1.59 (br s, 1H), 1.55-1.46 (m, 2H). Purity of the compound was further confirmed by RP-HPLC by using method 1: Rt=4.69 min (100% pure; FIG. 63).

Synthesis of Compound 22.

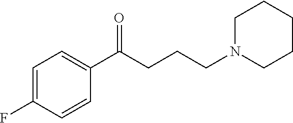 [SMILES: O=C(CCCN1CCCCC1)c1ccc(F)cc1]

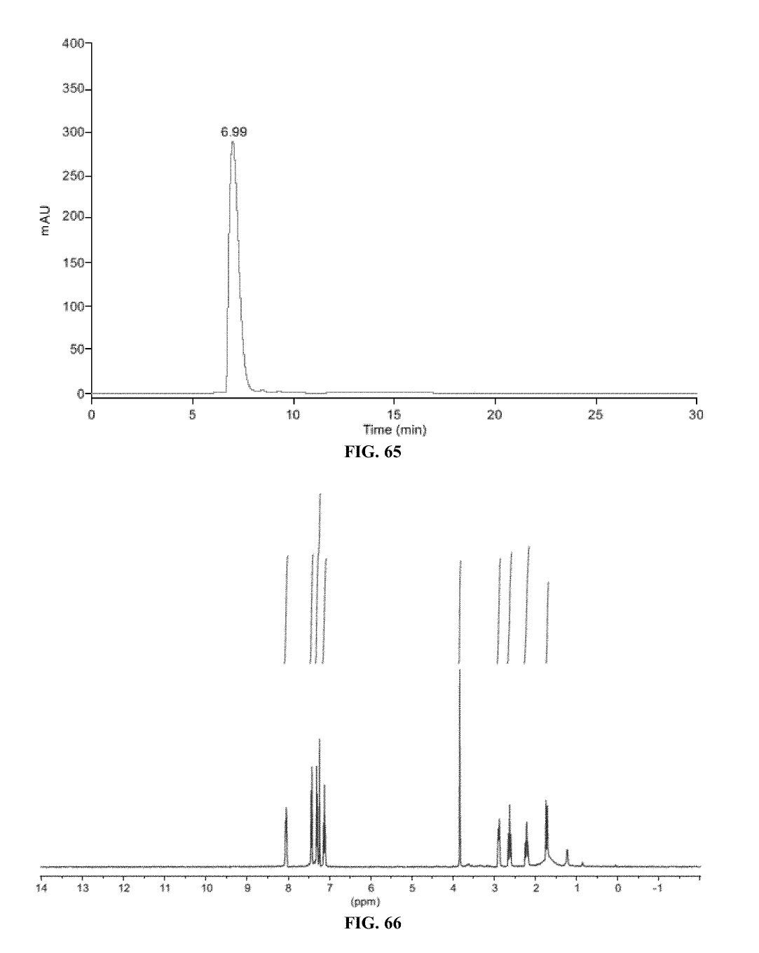

Following the general procedure described for the synthesis of compound 7, potassium iodide (0.96 g, 5.80 mmol), potassium carbonate (1.22 g, 8.81 mmol), 4′-fluoro-4-chlorobutyrophenone (0.88 g, 4.39 mmol), and piperidine (0.24 g, 2.87 mmol) in toluene (15 mL) were used to afford the known compound 22 (0.12 g, 17%, Rf 0.15 in MeOH:CH2Cl2/5:95) as a yellow oil: ¹H NMR (400 MHz, CDCl₃, which matches lit.⁴, FIG. 64) δ 7.98 (dd, J=8.8, 5.6 Hz, 2H), 7.10 (app. t, J=8.8 Hz, 2H), 2.94 (t, J=7.2 Hz, 2H), 2.35 (t, J=7.2 Hz, 6H), 1.92 (p, J=7.2 Hz, 2H), 1.52 (p, J=5.2 Hz, 4H), 1.39 (p, J=5.2 Hz, 2H). Purity of the compound was further confirmed by RP-HPLC by using method 1: Rt=6.99 min (100% pure; FIG. 65).

Synthesis of Compound 23.

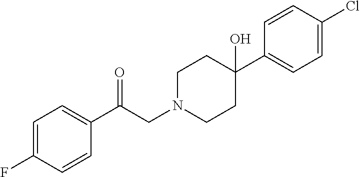 [SMILES: O=C(CN1CCC(O)(c2ccc(Cl)cc2)CC1)c1ccc(F)cc1]

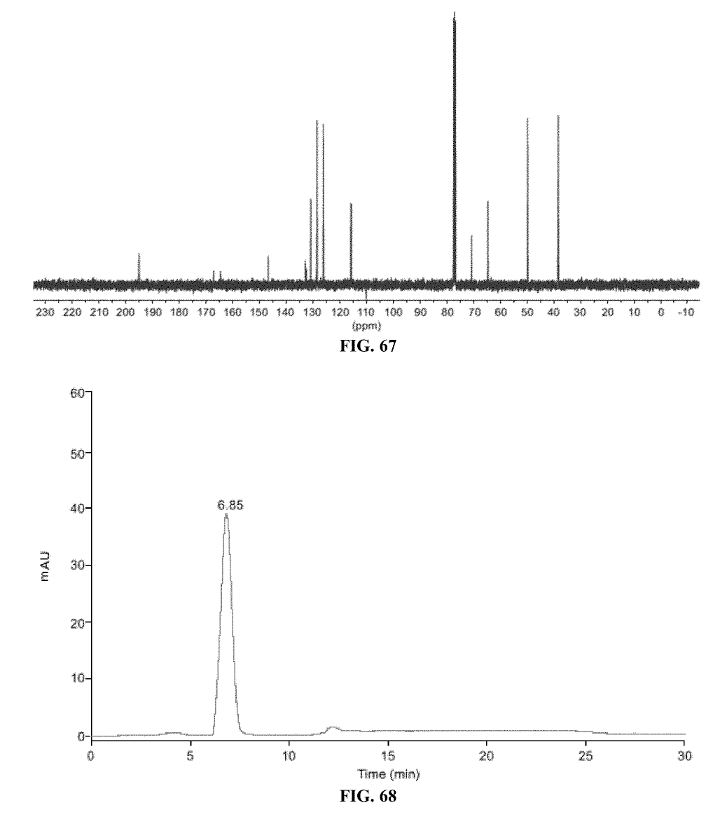

Following the general procedure described for the synthesis of compound 7, potassium carbonate (0.05 g, 0.36 mmol), 4′-fluoro-2-chloroacetophenone (0.04 g, 0.23 mmol), and 4-(4-chlorophenyl)-4-hydroxypiperidine (0.05 g, 0.23 mmol) in EtOH (10 mL) were used to afford compound 23 (0.067 g, 100%, Rf 0.33 in MeOH:CH2Cl2/5:95) as a white solid: ¹H NMR (400 MHz, CDCl₃, FIG. 66) δ 8.05 (dd, J=7.6, 5.6 Hz, 2H), 7.43 (d, J=8.4 Hz, 2H), 7.30 (d, J=7.6 Hz, 2H), 7.12 (app. t, J=8.4 Hz, 2H), 3.83 (s, 2H), 2.88 (m, 2H), 2.62 (t, J=11.6 Hz, 2H), 2.21 (td, J=11.6, 4.4 Hz, 2H), 1.72 (m, 2H), 1.71 (br s, 1H); ¹³C NMR (100 MHz, CDCl₃, FIG. 67) δ 195.0, 167.1, 164.5, 146.7, 132.9, 132.4, 130.9, 130.8, 128.4, 126.1, 115.8, 115.6, 70.7, 64.5, 49.7, 38.3; LRMS m/z calcd for _{C19H20ClFNO2} [M+H]⁺: 348.1; found 348.7. Purity of the compound was further confirmed by RP-HPLC by using method 1: Rt=6.85 min (95% pure; FIG. 68).

Synthesis of Compound 24.

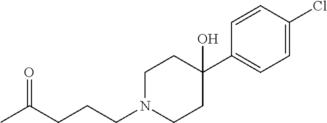 [SMILES: CC(=O)CCCN1CCC(O)(c2ccc(Cl)cc2)CC1]

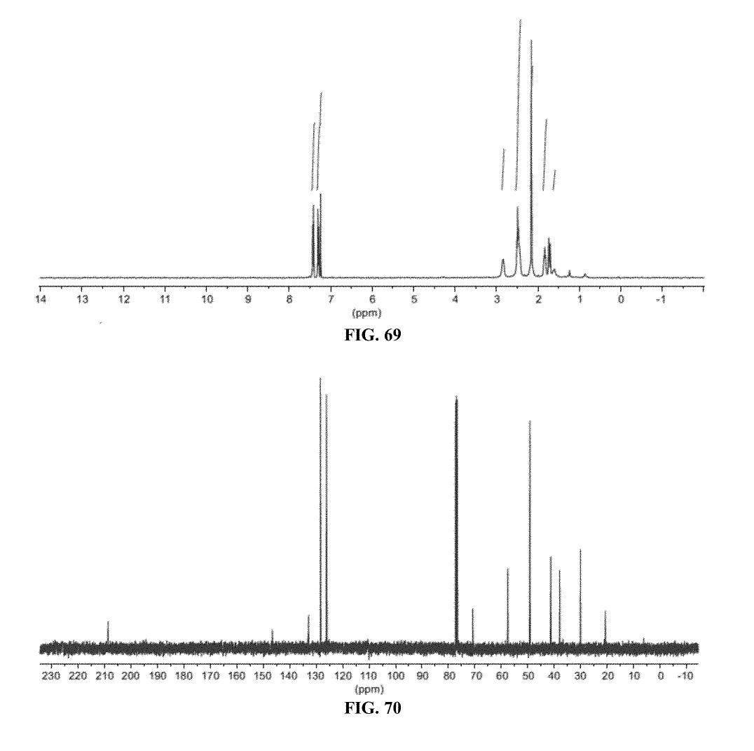

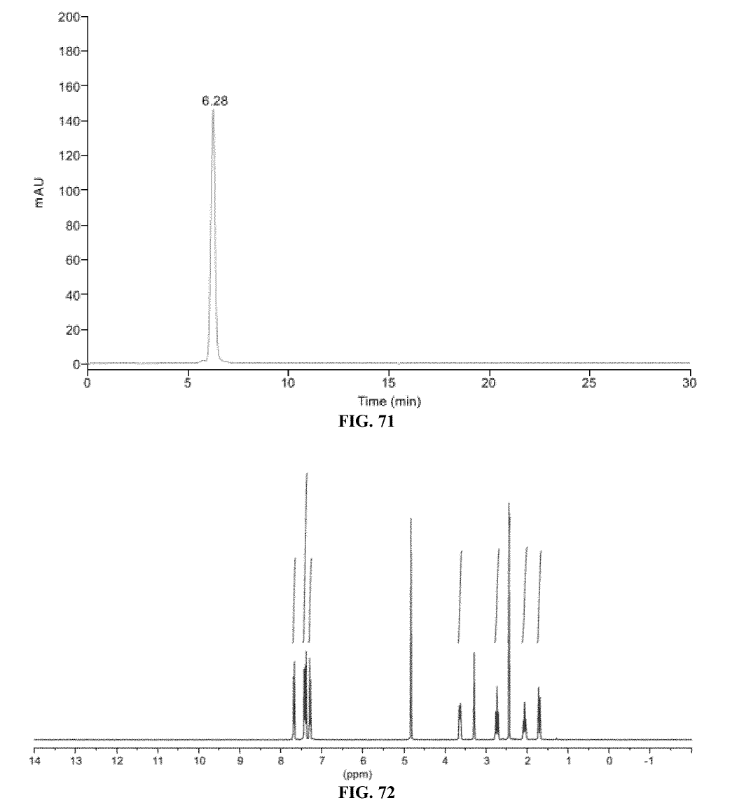

Following the general procedure described for the synthesis of compound 7, potassium iodide (0.55 g, 3.33 mmol), potassium carbonate (0.69 g, 5.00 mmol), 5-chloropentan-2-one (85% technical grade, 0.20 g, 1.41 mmol), and 4-(4-chlorophenyl)-4-hydroxypiperidine (0.70 g, 3.31 mmol) in toluene (20 mL) were used to afford compound 24 (0.044 g, 11%, Rf 0.10 in MeOH:CH2Cl2/5:95) as a light yellow solid: ¹H NMR (400 MHz, CDCl₃, FIG. 69) δ 7.42 (d, J=8.4 Hz, 2H), 7.29 (d, J=8.4 Hz, 2H), 2.88-2.79 (m, 2H), 2.48 (t, J=7.2 Hz, 2H), 2.50-2.40 (m, 4H), 2.21-2.12 (m, 2H), 2.15 (s, 3H), 1.83 (p, J=7.2 Hz, 2H), 1.72 (m, 2H), 1.60 (br s, 1H); 13C NMR (100 MHz, CDCl₃, FIG. 70) δ 208.6, 146.6, 132.8, 128.4, 126.1, 70.8, 57.6, 49.2, 41.3, 38.0, 30.1, 20.7; LRMS m/z calcd for _{C16H23ClNO2} [M+H]⁺: 296.1; found 296.7. Purity of the compound was further confirmed by RP-HPLC by using method 1: Rt=6.28 min (100% pure; FIG. 71).

Synthesis of Compound 25.

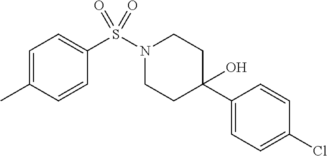 [SMILES: Cc1ccc(S(=O)(=O)N2CCC(O)(c3ccc(Cl)cc3)CC2)cc1]

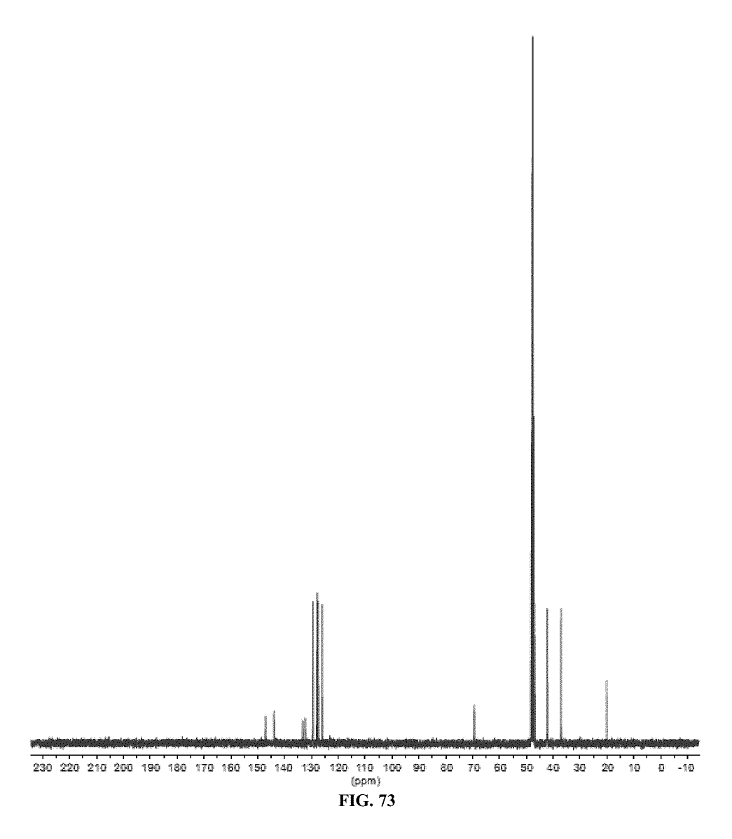

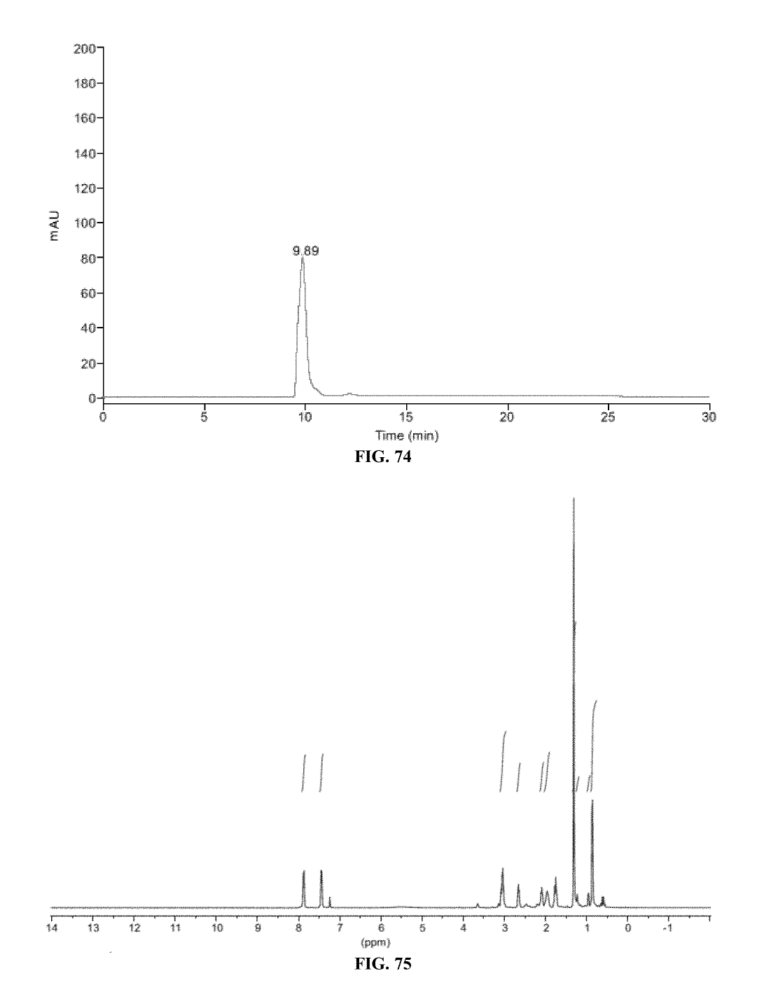

Following the general procedure described for the synthesis of compound 7, sodium carbonate (0.050 g, 0.472 mmol), p-TsCl (0.045 g, 0.236 mmol), and 4-(4-chlorophenyl)-4-hydroxypiperidine (0.050 g, 0.236 mmol) in MeCN (2 mL) were used to afford compound 25 (0.052 g, 60%, Rf 0.83 in MeOH:CH2Cl2/5:95) as a white solid: ¹H NMR (400 MHz, CD₃OD, FIG. 72) δ 7.70 (d, J=8.0 Hz, 2H), 7.44 (d, J=8.0 Hz, 2H), 7.42 (d, J=8.0 Hz, 2H), 7.31 (d, J=8.0 Hz, 2H), 3.67-3.64 (m, 2H), 2.75 (t, J=12.0 Hz, 2H), 2.08 (td, J=13.2, 4.4 Hz, 2H), 1.74-1.70 (m, 2H); ¹³C NMR (100 MHz, CD₃OD, FIG. 73) δ 148.6, 145.4, 134.8, 133.9, 131.0, 129.4, 129.0, 127.6, 70.9, 43.7, 38.6, 21.6; LRMS m/z calcd for _{C18H21ClNO3S} [M+H]⁺: 366.1; found 366.7. Purity of the compound was further confirmed by RP-HPLC by using method 1: Rt=9.89 min (99% pure; FIG. 74).

Synthesis of Compound 26.

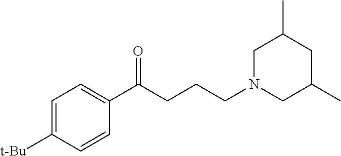 [SMILES: CC1CC(C)CN(CCCC(=O)c2ccc(C(C)(C)C)cc2)C1]

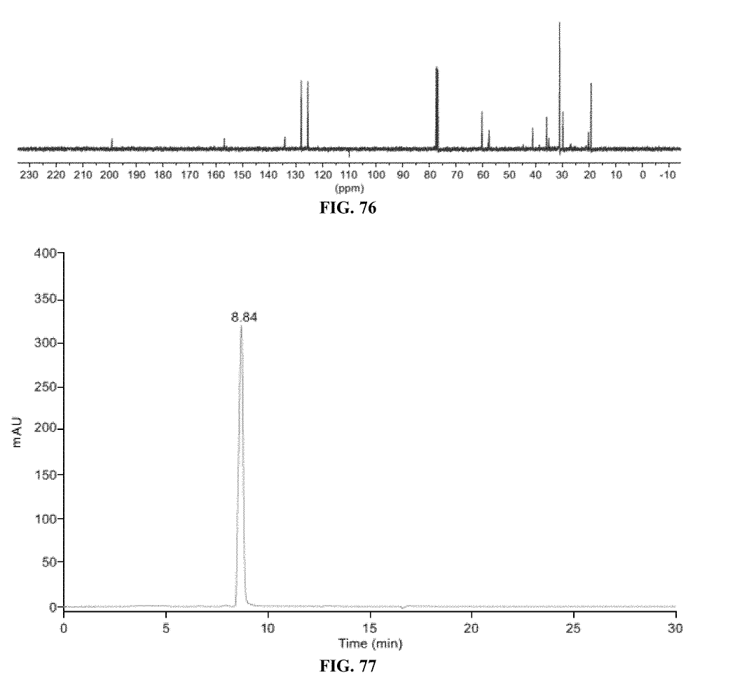

Following the general procedure described for the synthesis of compound 7, potassium iodide (0.58 g, 3.52 mmol), potassium carbonate (0.73 g, 5.30 mmol), 1-(4-(tert-butyl)phenyl)-3-chloropropan-1-one (0.63 g, 2.65 mmol), and 3,5-dimethylpiperidine (0.20 g, 1.76 mmol) in toluene (20 mL) were used to afford compound 26 (0.068 g, 24%, Rf 0.45 in MeOH:CH2Cl2/5:95) as a white solid: ¹H NMR (400 MHz, CDCl₃, FIG. 75) δ 7.87 (d, J=8.0 Hz, 2H), 7.44 (d, J=8.0 Hz, 2H), 3.07-3.02 (m, 4H), 2.65 (t, J=7.2 Hz, 2H), 2.09 (p, J=7.2 Hz, 2H), 2.02-1.90 (m, 3H), 1.75 (m, 3H), 1.31 (s, 9H), 0.86 (d, J=6.8 Hz, 6H); ¹³C NMR (100 MHz, CDCl₃, FIG. 76) δ 199.1, 156.9, 134.1, 128.0, 125.5, 60.1, 57.5, 41.1, 35.9, 35.1, 31.0, 29.8, 20.2, 19.2; LRMS m/z calcd for _{C21H34NO} [M+H]⁺: 316.3; found 316.9. Purity of the compound was further confirmed by RP-HPLC by using method 1: Rt=8.84 min (100% pure; FIG. 77).

Synthesis of Compound 27.

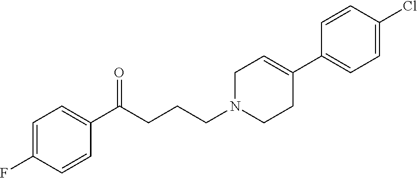 [SMILES: O=C(CCCN1CC=C(c2ccc(Cl)cc2)CC1)c1ccc(F)cc1]

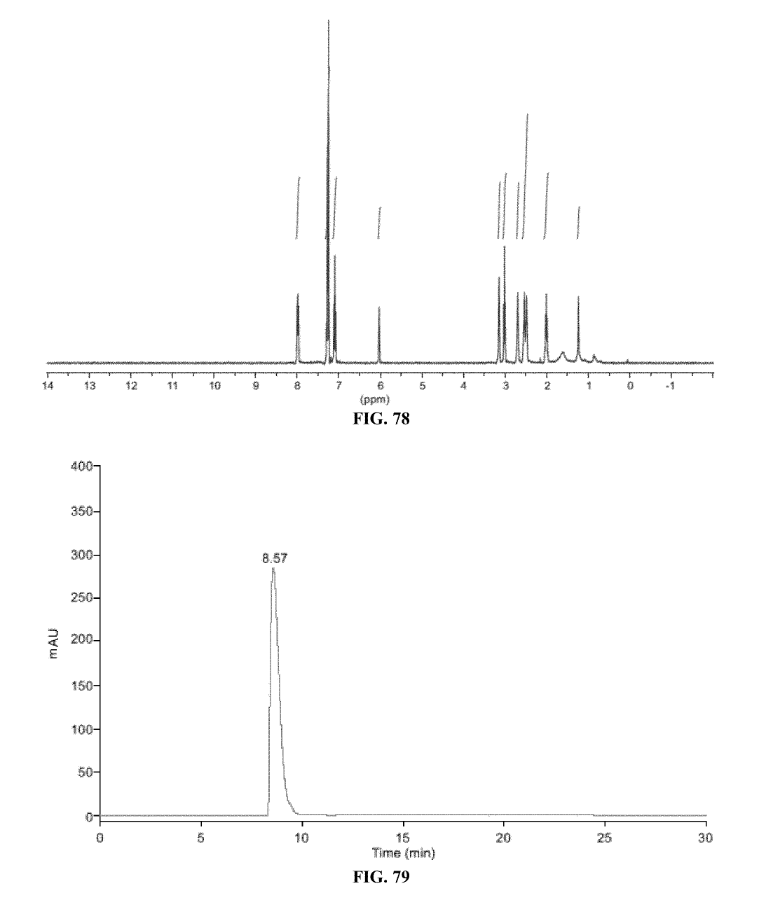

Compound 7 (0.38 g, 1 mmol) was dissolved in AcOH (10 mL) and HCl (2 mL). The reaction mixture was heated at reflux for 24 h, and then allowed to cool down to room temperature. KOH was added gradually until pH 8. The organic material was extracted with two portions of EtOAc (30 mL). The organic solvents were collected, dried using anhydrous MgSO4, and evaporated under reduced pressure. The obtained solid was purified by flash column chromatography (SiO2, MeOH:CH2Cl2/3:97, Rf 0.44) to yield compound 27 (0.30 g, 84% yield) as a white solid: ¹H NMR (400 MHz, CDCl₃, which matches lit.⁵, FIG. 78) δ 7.98 (dd, J=8.4, 6.0 Hz, 2H), 7.29-7.24 (m, 4H), 7.09 (app. t, J=8.4 Hz, 2H), 6.03 (m, 1H), 3.18-3.12 (m, 2H), 3.01 (t, J=7.2 Hz, 2H), 2.73-2.66 (m, 2H), 2.54 (t, J=6.4 Hz, 2H), 2.50-2.44 (m, 2H), 2.00 (p, J=7.2 Hz, 2H). Purity of the compound was further confirmed by RP-HPLC by using method 1: Rt=8.57 min (100% pure; FIG. 79).

Synthesis of Compound 28.

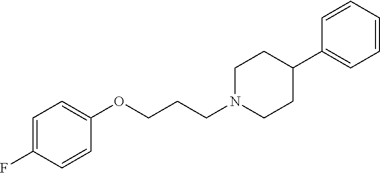 [SMILES: Fc1ccc(OCCCN2CCC(c3ccccc3)CC2)cc1]

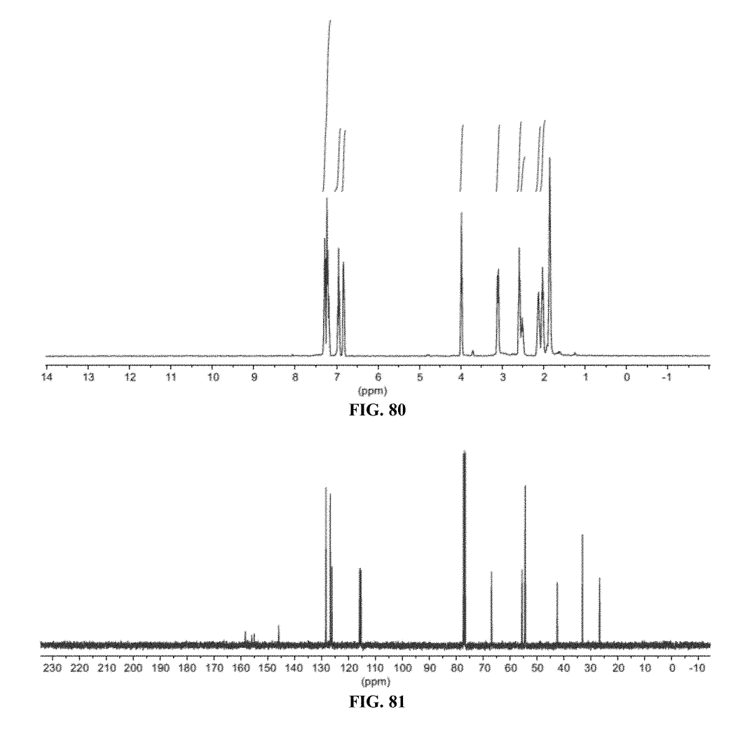

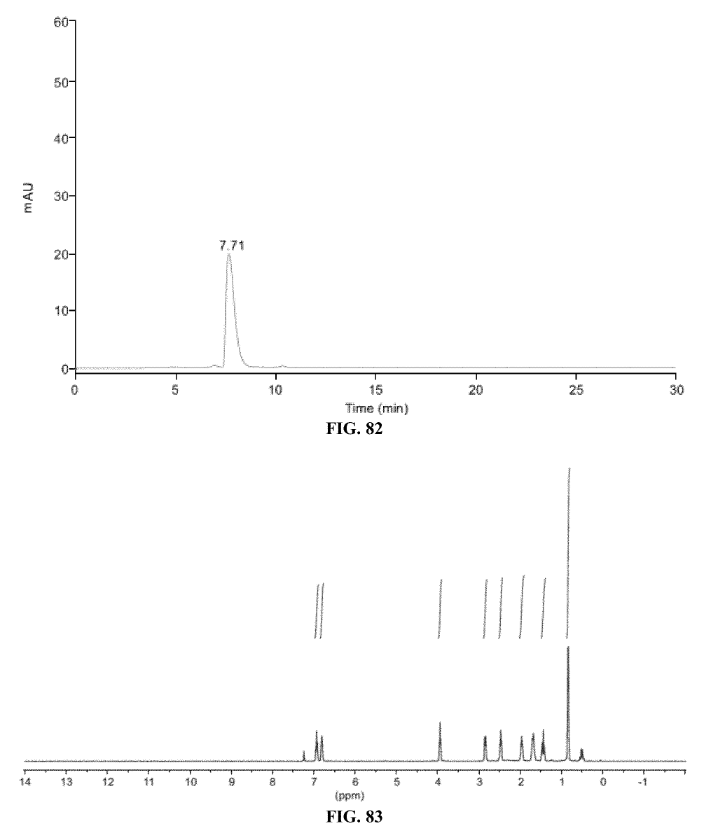

Following the general procedure described for the synthesis of compound 7, potassium iodide (0.12 g, 0.72 mmol), potassium carbonate (0.26 g, 1.83 mmol), 3-(4-fluorophenoxy)propyl chloride (0.17 g, 0.91 mmol), and 4-phenylpiperidine (0.15 g, 0.91 mmol) in THF (10 mL) were used to afford compound 28 (0.13 g, 45%, Rf 0.59 in MeOH:CH2Cl2/1:9) as a white solid: ¹H NMR (400 MHz, CDCl₃, FIG. 80) δ 7.29 (app. t, J=7.2 Hz, 2H), 7.23-7.17 (m, 3H), 6.95 (t, J=8.4 Hz, 2H), 6.84-6.80 (m, 2H), 3.98 (t, J=6.0 Hz, 2H), 3.12-3.09 (m, 2H), 2.59 (t, J=7.6 Hz, 2H), 2.52 (p, J=7.6 Hz, 1H), 2.16-2.09 (m, 2H), 2.02 (p, J=6.0 Hz, 2H), 1.88-1.81 (m, 4H); ¹³C NMR (100 MHz, CDCl₃, FIG. 81) δ 158.4, 156.0, 155.0, 146.0, 128.4, 126.8, 126.2, 115.9, 115.6, 115.5, 115.4, 66.9, 55.5, 54.4, 42.5, 33.1, 26.7; LRMS m/z calcd for C20H25FNO [M+H]⁺: 314.2; found 314.8. Purity of the compound was further confirmed by RP-HPLC by using method 1: Rt=7.71 min (99% pure; FIG. 82).

Synthesis of Compound 29.

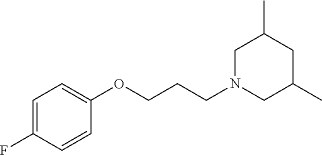 [SMILES: CC1CC(C)CN(CCCOc2ccc(F)cc2)C1]

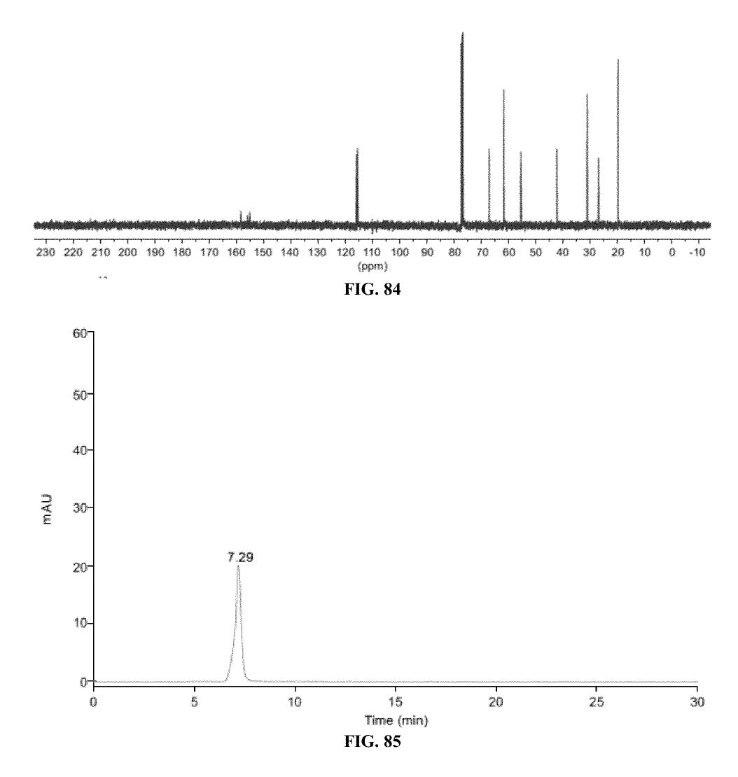

Following the general procedure described for the synthesis of compound 7, potassium iodide (0.03 g, 0.18 mmol), potassium carbonate (0.21 g, 1.54 mmol), 3-(4-fluorophenoxy)propyl chloride (0.15 g, 0.77 mmol), and 3,5-dimethylpiperidine (0.09 g, 0.77 mmol) in THF (10 mL) were used to afford compound 29 (0.022 g, 10%, Rf 0.56 in MeOH:CH2Cl2/1:9) as a white solid: ¹H NMR (400 MHz, CDCl₃, FIG. 83) δ 6.93 (app. t, J=8.0 Hz, 2H), 6.83-6.77 (m, 2H), 3.94 (t, J=6.8 Hz, 2H), 2.86-2.83 (m, 2H), 2.47 (t, J=6.8 Hz, 2H), 1.96 (p, J=6.8 Hz, 2H), 1.69 (d, J=9.6 Hz, 4H), 1.44 (t, J=6.8 Hz, 2H), 0.83 (d, J=5.6 Hz, 6H); ¹³C NMR (100 MHz, CDCl₃, CDCl₃, FIG. 84) δ 158.3, 156.0, 155.1, 115.8, 115.6, 115.5, 115.4, 67.1, 61.7, 55.4, 42.1, 31.0, 26.8, 19.6; LRMS m/z calcd for _{C16H25FNO} [M+H]⁺: 266.2; found 266.8. Purity of the compound was further confirmed by RP-HPLC by using method 1: Rt=7.29 min (100% pure; FIG. 85).

Synthesis of Compound 30.

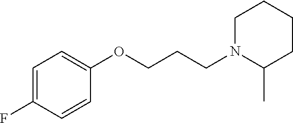 [SMILES: CC1CCCCN1CCCOc1ccc(F)cc1]

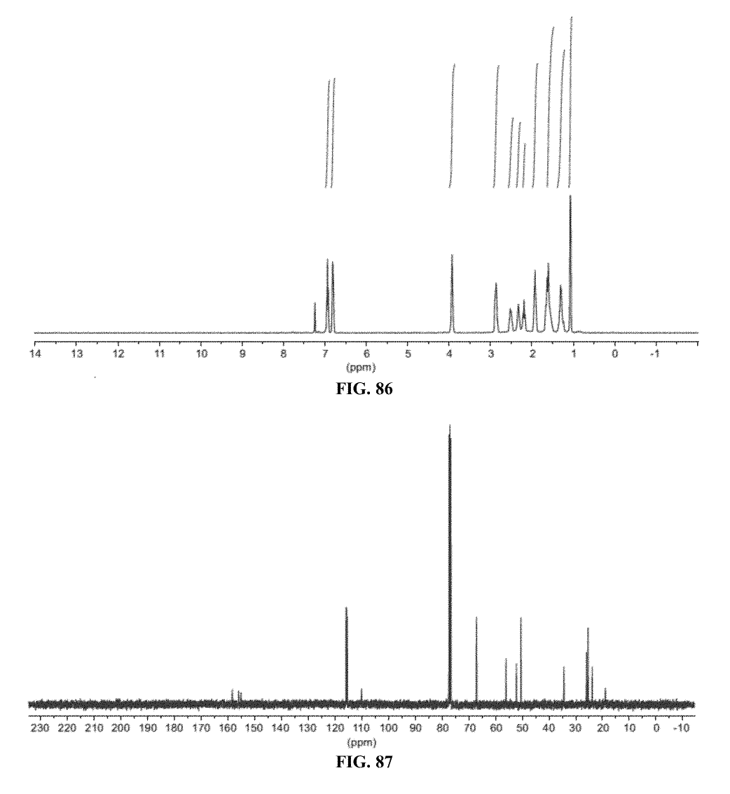

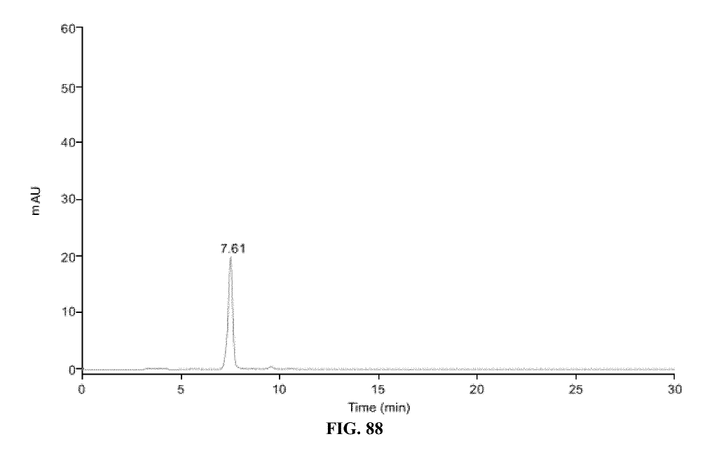

Following the general procedure described for the synthesis of compound 7, potassium iodide (0.03 g, 0.18 mmol), potassium carbonate (0.21 g, 1.54 mmol), 3-(4-fluorophenoxy)propyl chloride (0.15 g, 0.77 mmol), and 2-methylpiperidine (0.08 g, 0.77 mmol) in THF (10 mL) were used to afford compound 30 (0.012 g, 6%, Rf 0.30 in MeOH:CH2Cl2/1:9) as a white solid: ¹H NMR (400 MHz, CDCl₃, FIG. 86) δ 6.94 (app. t, J=7.6 Hz, 2H), 6.83-6.77 (m, 2H), 3.93 (m, 2H), 2.892.83 (m, 2H), 2.54-2.48 (m, 1H), 2.22-2.38 (m, 1H), 2.19 (t, J=10.8 Hz, 1H), 1.92 (p, J=6.8 Hz, 2H), 1.67-1.50 (m, 4H), 1.35-1.24 (m, 2H), 1.07 (d, J=4.4 Hz, 3H); ¹³C NMR (100 MHz, CDCl₃, FIG. 87) δ 158.3, 156.0, 155.1, 115.8, 115.6, 115.5, 115.4, 67.1, 56.0, 52.2, 50.5, 34.4, 26.0, 25.4, 23.8, 18.9; LRMS m/z calcd for _{C15H23FNO} [M+H]⁺: 252.2; found 252.8. Purity of the compound was further confirmed by RP-HPLC by using method 1: Rt=7.61 min (98% pure; FIG. 88).

### Example 4

Candida spp. are a normal part of the skin and gastrointestinal microbiome. However, under certain conditions they can cause infections. Patients who are immunocompromised, who are being treated with broad-spectrum antibiotics, who have diabetes, who are being treated with corticosteroids, and who have burn wounds are more prone to fungal infections. On the skin and mucosal membranes, Candida spp. can cause cutaneous infections, which when untreated can lead to invasive infections affecting the eyes, heart, and brain with a high mortality rate. In addition, Candida spp. are the primary cause of oral thrush and vulvovaginal infections.

As Candida spp. are eukaryotes, development of antifungal drugs has exploited differences in cell membrane structure. There are 3 classes of drugs used to treat Candida infections. These classes include the azoles, the polyenes, and the echinocandins. The polyenes are broad-spectrum antifungals and include nystatin (NYT) topical cream and amphotericin B (AMB), which is only delivered intravenously. The echinocandins are narrow-spectrum antifungals, and the only approved member of this antifungal family for topical application is micafungin. The azoles are broad-spectrum antifungals that are used to treat Candida infections by inhibiting ergosterol biosynthesis.

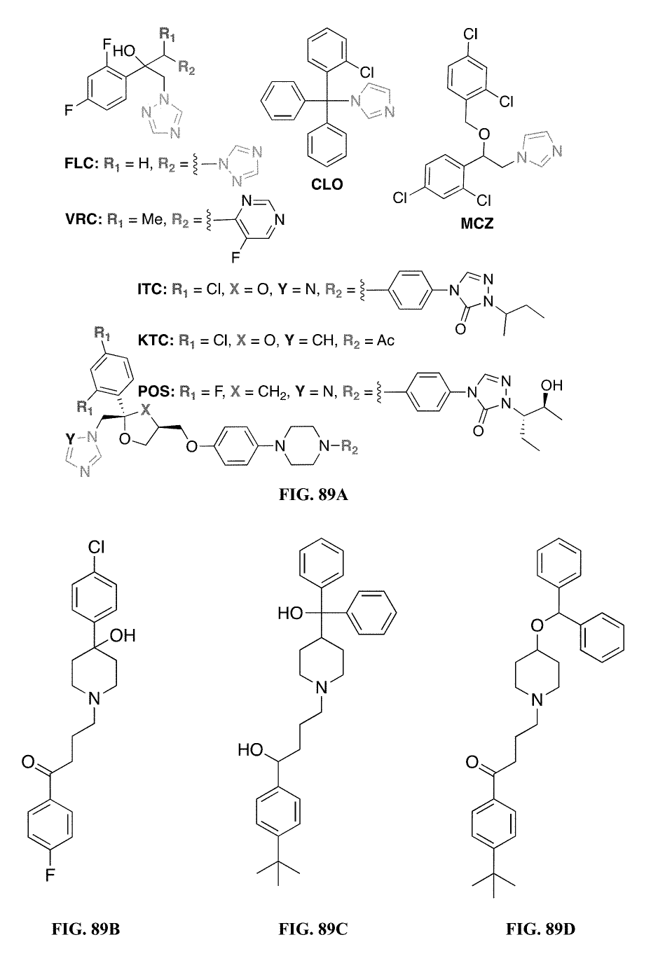

The azoles are comprised of imidazoles and triazoles. The imidazoles, clotrimazole (CLO), miconazole (MCZ), and ketoconazole (KTC), are used to treat skin infections while the triazoles, fluconazole (FLC), itraconazole (ITC), posaconazole (POS), and voriconazole (VRC), are used for invasive infections (FIG. 89A). For cutaneous infections, treatment with azoles occurs for two weeks or until the infection clears and skin irritation can occur. In addition, there are reports of infections resistant to azoles, especially in non-albicans Candida strains.

Previous studies have shown the antipsychotic haloperidol (FIG. 89B) and its derivatives to act synergistically with azole antifungals in vitro. Some antihistamines such as terfenadine (TERF) (FIG. 89C) and ebastine (EBA) (FIG. 89D) have a 4-piperidinol group as well as an acetophenone moiety similar to haloperidol, but are not able to cross the blood-brain barrier to cause drowsiness. Interestingly, studies have explored TERF for additional drug activities, including antibacterial and antitumor activity.

This Example explores novel combinations of antifungals and antihistamine drugs as synergistic antifungal combinations for potential use as topical antifungal treatments. 2 antihistamines, TERF and EBA, were investigated by checkerboard dilution assays with 7 azole antifungal drugs, CLO, KTC, MCZ, ITC, POS, FLC, and VRC against a panel of 13 Candida spp. Combinations with successful fractional inhibitory concentration index (FICI) scores were then evaluated in time-kill assays, biofilm disruption assays, and mammalian cytotoxicity assays to assess the cytotoxicity of the drug combinations with planktonic fungal cells, sessile fungal cells, and mammalian cells, respectively.

It was found that 55 out of 91 combinations tested of TERF and EBA against the various fungal strains were synergistic with the azoles. These findings suggest a potential new strategy for targeting drug-resistant Candida infections.

### Results

Determination of Synergistic Azole Antifungal and Antihistamine Combinations by Checkerboard Assays.

In order to establish the concentration ranges to use for combination studies of azoles and antihistamines, the minimal inhibitory concentration (MIC) values of 7 azole antifungals (3 imidazoles: CLO, KTC, and MCZ; as well as 4 triazoles: ITC, POS, FLC, and VRC) and 2 antihistamine agents (TERF and EBA) were individually determined against 13 yeast strains (strains A-M), of which one strain is annotated as FLC sensitive (strain E) and all other are considered FLC resistant (TABLES 6 and 7). Many of the strains were susceptible to the imidazole antifungals CLO, KTC, and MCZ (TABLE 6), but showed resistance to the triazole antifungals ITC, POS, FLC, and VRC (TABLE 7). The antihistamines did not show any antifungal activity against the 13 strains tested.

The potential synergy of azole and antihistamine combinations was investigated next by using checkerboard assays (TABLES 6 and 7). Combinations were considered synergistic (SYN) if they displayed FICI≤0.5, additive (ADD) if 0.5<FICI≤2, and antagonistic (ANT) if FICI≥4. Under this definition, both drugs need to have at least a 4-fold decrease in MIC value when used in combination as compared to their MIC alone to be considered a synergistic combination. To further define combinations where inhibition of growth, but no synergy was observed, the term partial synergy (pSYN) was used for combinations where one of the drugs displayed at least a 4-fold decrease in MIC value while the other drug displayed a 2-fold decrease in MIC value, resulting in 0.5<FICI≤0.75. Furthermore, for 0.75<FICI≤1.5, in cases where one drug showed at least a 2-fold decrease in MIC value in combination, a strong additive effect (ADD*) was considered. As the antihistamines showed no activity against the fungal strains tested, and many fungal strains were resistant to multiple azole antifungals, there are drugs with MIC values greater than the maximum concentration of drugs tested. In these cases, the maximum concentration of drug tested was considered the MICₐₗₒₙₑ, which results in larger FICI values.

The combination with the best FICI score was POS and EBA against C. glabrata (strain H) with an FICI value of 0.05. Strain H is resistant to both POS and EBA alone, having MIC values of >32 and >25 μg/mL, respectively. In combination, 0.5 μg/mL of POS and 0.78 μg/mL of EBA inhibited the growth of strain H, which is a 64-fold decrease in POS and a 32-fold decrease in EBA. Other combinations with FICI values lower than 0.10 included POS and TERF as well as KTC, MCZ, and VRC with EBA, all against C. glabrata (strain H). While strain H appears very sensitive to both antihistamines and these POS and EBA combinations, this is interesting as C. glabrata is a species that tends to be naturally resistant to many azoles. With strain H, there were a total of 5 SYN combinations with TERF and 6 with EBA. In contrast, the clinical isolates of C. glabrata (CG1-CG3; strain I-K) tended to show more resistance, but there were still 6 cases of SYN with strain I (5 with TERF, 1 with EBA), 2 SYN with strain J (2 with TERF), and 3 cases with strain K (3 with TERF). The other non-albicans Candida that showed many cases of synergy with EBA was C. parapsilosis (strain M) with 5 cases of SYN and 2 cases of pSYN. While multiple combinations with EBA produced very low FICI values, overall, there are many more cases of synergy with TERF across all the azole antifungals and all fungal strains.

In total, there are 41 cases of SYN and 22 cases of pSYN with TERF. For EBA, there is a smaller number of synergistic combinations with 14 cases of SYN and 9 cases of pSYN. It appears that the synergistic effect of EBA is more strain-dependent. In addition, POS appears to have the best effect with EBA against many strains. For both TERF and EBA, combinations with POS and ITC appear to be very effective against C. krusei (strain L), C. parapsilosis (strain M), and C. glabrata (strain H). However, there are still many cases of pSYN for TERF with FLC and against C. albicans ATCC MYA-1003 (strain A). There were many cases of SYN with C. albicans strains in azole and TERF combinations, especially in strains C, D, E, and G, all of which are resistant to most triazoles. In contrast to TERF, EBA appeared to be best with C. parapsilosis (strain M). For a summary of FICI scores, the checkerboard results are presented in heat map style below (TABLES 8 and 9).

Time-Dependent Killing of Fungi with POS and EBA Combinations.

To examine the efficiency of azole and antihistamine combinations at inhibiting the growth of fungi, the POS and EBA combination was tested against 2 yeast strains, C. albicans ATCC 10231 (strain B) and C. glabrata ATCC 2001 (strain H). As EBA is approved in many countries and as of all the azoles tested POS had the highest instances of synergy and the lowest FICI score with C. glabrata (strain H), POS and EBA combinations were selected as representatives for the time-kill assays as well as for the cytotoxicity and biofilm studies. To study the synergistic effect of POS and EBA, POS and EBA were tested alone at sub-inhibitory concentrations and compared these to POS and EBA combinations.

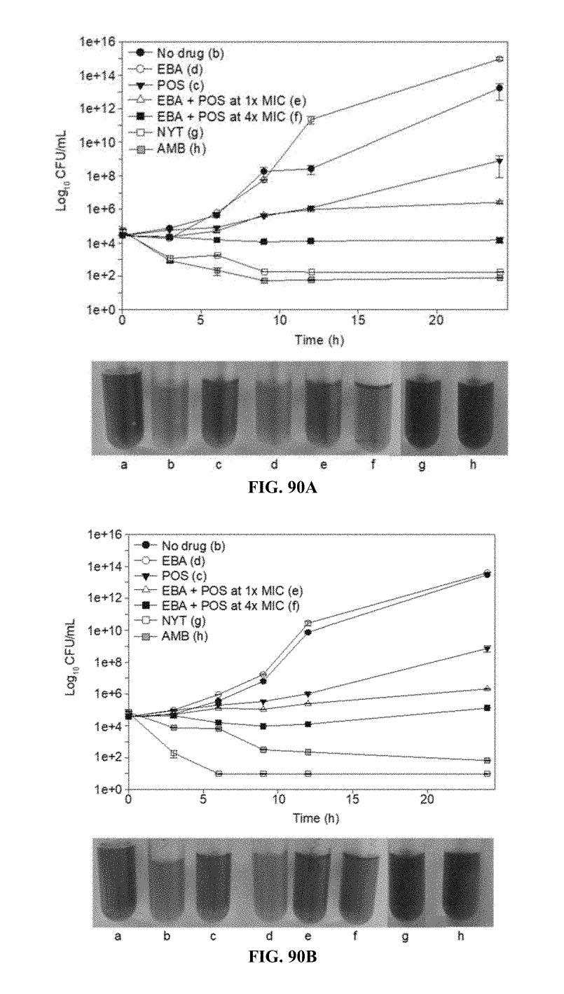

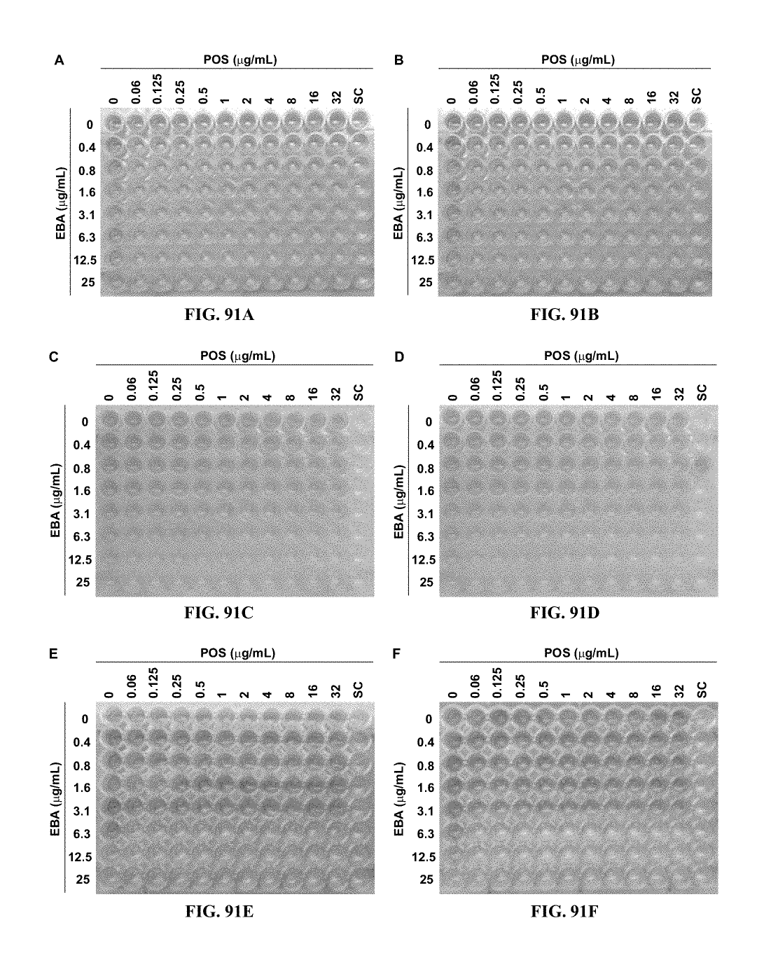

The specific experiments included no drug (growth control), POS alone at concentration used in MIC_{combination}, EBA alone at concentration used in MIC_{combination}, POS with EBA at MIC_{combination}, and POS with EBA at 4× the MIC_{combination}, and NYT and AMB at 1×MIC as fungicidal controls. Both strains B and H showed similar profiles in the relative amounts of colony forming units (CFU) over time (FIGS. 90A-B). EBA alone did not inhibit fungal growth. The sub-inhibitory concentration of POS partially inhibited the growth of strains B and H; only reaching 10⁸ CFU/mL at 24 h as compared to 10¹³ for each the growth control. In combination at 1×MIC, POS and EBA strongly inhibited fungal growth, limiting the growth to 10⁶ CFU/mL through the 24 h time period, but inhibition was not complete. However, at 4×MIC, counts remained between 10⁴ and 10⁵ CFU/mL throughout the 24-hour time frame, indicating complete inhibition of fungal growth and between 10² and 10³ decrease as compared to POS alone. Both NYT and AMB showed fungicidal activity by decreasing the initial inoculum of 10⁵ to 10¹ to 10² CFU/mL by 24 h.

Determination of Biofilm Disruption Activity by POS and EBA Combinations.

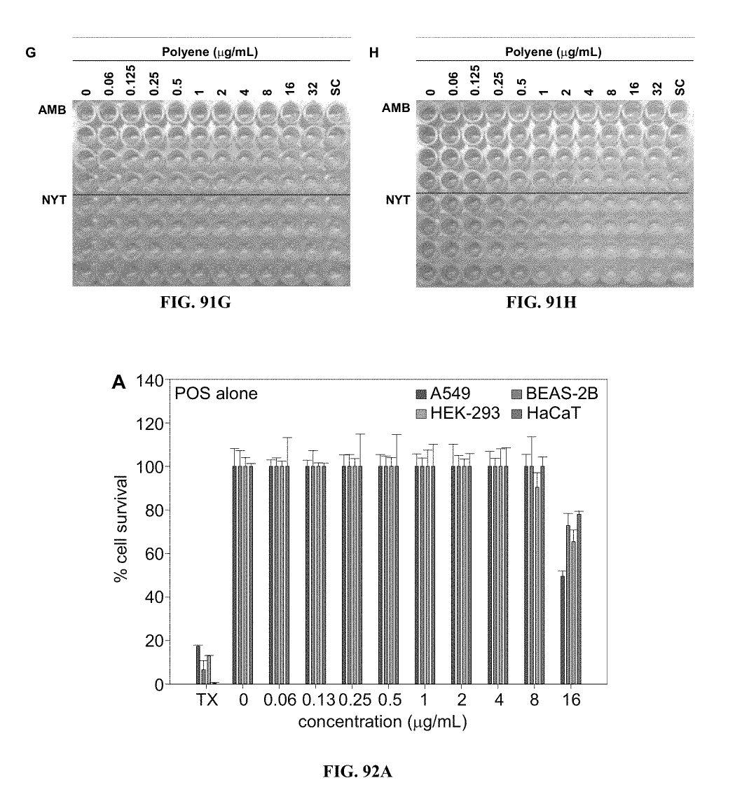

To evaluate the ability of POS and EBA combinations to disrupt biofilms (sessile MIC values (SMIC)), a cell viability assay using the water-soluble dye XTT in checkerboard format was performed (TABLE 10 and FIGS. 91A-F). Two C. albicans strains, ATCC 10231 (strain B) and ATCC 64124 (strain F), and one C. glabrata strain, ATCC 2001 (strain H), were chosen as representative strains. As free-floating planktonic cells, strain B is generally sensitive to azole antifungals, while both strains F and H are resistant. Against the biofilms, neither POS nor EBA showed any activity (SMIC₉₀ for POS>32 μg/mL and SMIC₉₀ for EBA≥25 μg/mL against strains B, F, and H). When tested in combination, POS with EBA had no activity against both C. albicans strains (B and F). However, POS with EBA showed synergy against C. glabrata (strain H) (SMIC₉₀ combo for POS=0.06 μg/mL and SMIC₉₀ combo for EBA=6.3 μg/mL). Similarly, both AMB and NYT had no activity against C. albicans (strain B), but had an SMIC₉₀ of 4 μg/mL against C. glabrata (strain H) (TABLE 11 and FIGS. 91G-H).

TABLE 11
SMIC values for polyenes against biofilms of 2 fungal strains.

| strain | Polyene | SMIC₉₀ |
|---|---|---|
| B | AMB | >32 |
| | NYT | >32 |
| H | AMB | 4 |
| | NYT | 4 |

Strains: B = C. albicans ATCC 10231, H = C. glabrata ATCC 2001.

Evaluation of Mammalian Cytotoxicity of POS and EBA Combinations.

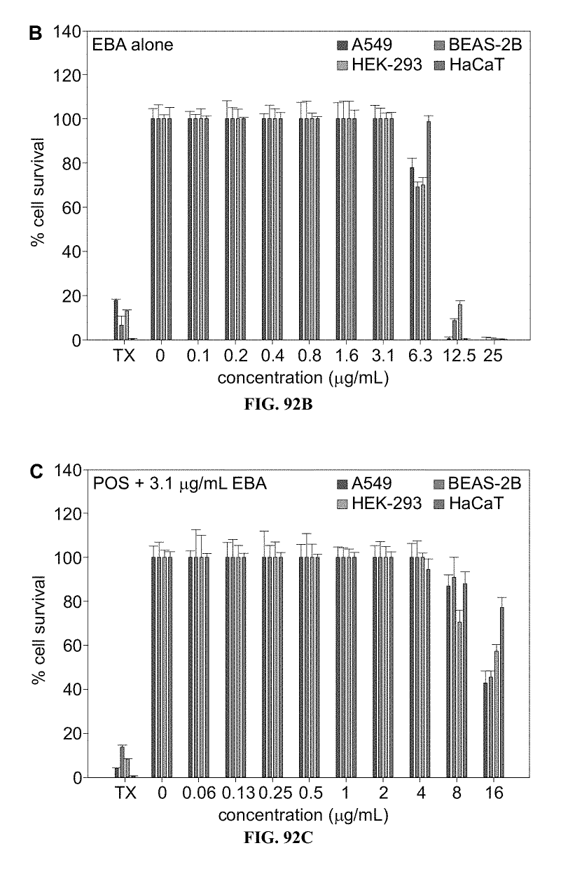

To examine whether the activity of POS and EBA combinations is specific to fungal strains, a resazurin cell viability assay was used to assess the cytotoxic effect of POS and EBA alone and in combination against mammalian cells (FIGS. 92A-C). For these assays, 4 human cell lines were used, including lung carcinoma epithelial cells (A549), bronchial epithelial cells (BEAS-2B), embryonic kidney cells (HEK-293), and of course, the keratinocytes (HaCaT), which are most relevant for skin infections. POS was tested within the concentration range of 0.06-16 μg/mL. With POS alone, the maximum concentration with no observed toxicity was 4 μg/mL for HEK-293 and A549, whereas it was 8 μg/mL for HaCaT and BEAS-2B. For EBA alone, concentrations ranging from 0.1-25 μg/mL were used. Complete cell survival was observed up to 6.3 μg/mL for HaCaT and 3.1 μg/mL for all other cell lines. A cytotoxic effect began at 6.3 μg/mL with cell survival of 78±8%, 69±5%, and 70±7% for A549, BEAS-2B, and HEK-293, respectively. At the highest concentration of EBA, 25 μg/mL, near 0% cell survival was observed for all cell lines.

To examine the effect of POS and EBA combinations on cytotoxicity, the concentration of POS was varied with a constant amount of EBA. Since all cell lines showed no toxicity at 3.1 μg/mL of EBA, POS was assessed with all cell lines in combination with 3.1 μg/mL of EBA (FIG. 92C). Cell survival rates of POS+3.1 μg/mL of EBA was not significantly different as compared to POS alone for all 4 cell lines.

### DISCUSSION

Candida infections primarily affect immunocompromised patients, but they also occur in otherwise healthy adults. It is estimated that 75% of all women will have at least one vulvovaginal infection during their life. Approximately half of vaginal infections are caused by Candida species. Out of the cases of candidiasis, a study in China reported that 80.5% of vaginal candidiasis cases over an 8-year period were caused by C. albicans, while 18%, 1.2%, and 0.1% were caused by C. glabrata, C. krusei, and C. tropicalis, respectively. In this Example, 55 synergistic combinations of azole antifungals and antihistamines were found. While these combinations did not reverse FLC resistance, there appeared to be an adjuvant effect with other azole antifungals. While both EBA and TERF showed synergy with azoles against C. albicans strains, each also showed synergy against non-albicans Candida strains, which tend to have more intrinsic resistance to azole antifungals. Furthermore, as many skin diseases have similar symptoms, diagnosis of fungal infections without a diagnostic test can be difficult. Due to this difficulty and the associated irritation from fungal infections, clotrimazole is also formulated with hydrocortisone or betamethasone, a highly potent fluorinated corticosteroid. However, corticosteroids used for prolonged periods and in occlusive environments such as where fungal infections normally occur, can have serious side effects including severe allergic reactions and skin atrophy. If combinations of azoles and antihistamines were shown to be efficacious in animal models and further testing, a topical cream containing an azole and an antihistamine could be an alternative product for the clotrimazole corticosteroid topical cream.

In addition to checkerboard assays, time-kill studies were used to further verify the synergistic interactions between the azoles and antihistamines. 2 fungal strains and the POS and EBA combination were chosen to observe the inhibitory effect of the drug combination over time. The results from the time-kill study do substantiate the synergy observed in the checkerboard assays. Additionally, as azoles are fungistatic against Candida spp., time-kill dynamics were studied to evaluate whether the combinations of azoles and antihistamines remained fungistatic. As the number of CFU/mL remained constant for POS and EBA in combination at 4×MIC while the fungicidal controls, AMB and NYT, decreased counts to ≤10² CFU/mL, this suggests that the POS and EBA combinations are fungistatic.

Many fungal strains in vivo can form biofilms, especially on implanted medical devices such as joint replacements and urinary catheters. Treatment of infections where biofilms occur is more challenging as the biofilms protect fungal cells from the antifungal drugs. In biofilms, fungal cells secrete an oligosaccharide layer that can prevent antifungal drugs from penetrating the biofilm and acting on the fungal cells. Biofilms remaining intact after treatment increases the risk of reoccurring infections. As an additional measure to evaluate the effectiveness of the POS and EBA combinations, a biofilm disruption assay was used to look at the outcome on sessile cells. While synergy against C. albicans biofilms was not observed, synergy against C. glabrata (strain H) biofilm was. The concentration of POS and EBA used to achieve SMIC₉₀ against strain H had similar concentrations of POS and about 100-fold less concentration of EBA as compared to the 4 μg/mL of AMB and NYT needed to also achieve SMIC₉₀. The difference in results between fungal species may be due to differences in the oligosaccharide composition of the biofilms or differences in regulation of efflux pumps.

Since the synergistic interaction was corroborated with POS and EBA, cytotoxicity was then assessed. It was found that EBA at 3.1 μg/mL in combination with POS has a similar cell survival rates as POS alone, which is non-toxic to the mammalian cells up to at least 16 μg/mL. The concentration of POS alone at which a cytotoxic effect was beginning to be observed, 16 μg/mL, was at significantly higher concentrations of POS than the MIC values for POS alone against sensitive fungal strains, as the MIC values for POS ranged from 0.25-1.0 μg/mL. In synergistic combinations, the MIC values for POS ranged from 0.008-0.5 μg/mL, while MIC values for EBA in synergistic combinations with POS ranged from 0.78-3.1 μg/mL. As the MIC values for POS and EBA in synergistic combinations are the same as or lower than the concentrations of drugs in combination where no cytotoxic effect was observed, it suggests that POS and EBA combinations would be safe to use.

The known targets for EBA and TERF are the histamine H1 receptors. In addition, TERF, and to a lesser extent EBA, is known to inhibit the human cardiac hERG potassium channel at high concentrations. While fungi also have potassium channels, there is no known orthologue to hERG in fungi. The mechanism of action of the antifungal activity is uncertain for TERF and EBA. However, other groups have proposed mechanisms of action for TERF with regards to the other activities it exhibits. While TERF was not tested for cytotoxicity, reports show that TERF and its derivative have strong antitumor activity, suggesting that TERF would be cytotoxic at the concentrations tested. In vitro, TERF sensitized cancer cells to doxorubicin by a proposed mechanism of inhibiting P-glycoprotein. Other studies have proposed TERF as a CYP2J2, inhibitor, which also plays a role in apoptosis and inhibiting cancer cells. Furthermore, TERF and its derivatives show antibacterial activity against Staphylococcus aureus, which may be partially due to inhibition of type II topoisomerases. Less work has been done to identify secondary targets of EBA, but one study showed in silico evidence as an ATPase inhibitor. As TERF and EBA are structurally similar, it is believed that both compounds have similar targets.

In sum, the effectiveness of combinations of 7 azole antifungals and 2 antihistamines, EBA and TERF, was evaluated against a wide panel of fungal strains. Both TERF and EBA were found to exhibit synergy in combination with azole antifungals. The majority of the cases of synergy were observed with POS and VRC and specific strains of Candida. More synergistic combinations were observed with TERF than EBA, however, the POS and EBA combination with C. glabrata (strain II) had the lowest FICI value. By time-kill studies it was found that POS and EBA at 1×MIC were fungistatic. The POS and EBA combination was also found to be synergistic against biofilms of C. glabrata (strain H). Finally, POS and EBA combinations were shown not to be toxic to mammalian cells. Overall, this work demonstrated that azoles and antihistamines combinations are useful for targeting topical fungal infections.

### MATERIALS AND METHODS

Antifungal and Antihistamine Agents Used for Combination Studies.

The antifungal agents fluconazole (FLC), itraconazole (ITC), posaconazole (POS), and voriconazole (VRC) were purchased from AK Scientific (Union City, Calif., USA). The remaining antifungal agents amphotericin B (AMB), clotrimazole (CLO), ketoconazole (KTC), miconazole (MCZ), and nystatin (NYT) were purchased from Sigma-Aldrich (St. Louis, Mo., USA). The antihistamines used were terfenadine (TERF, Sigma-Aldrich, St. Louis, Mo., USA) and ebastine (EBA, VWR, Atlanta, Ga., USA). All compounds were dissolved in DMSO (Sigma-Aldrich, St. Louis, Mo., USA) for use in assays. It is to note that all other chemicals used for the various experiments were purchased from Sigma-Aldrich (St. Louis, Mo., USA) and used without any further purification.

Fungal Strains, Mammalian Cell Lines, and their Culture Conditions.

The Candida albicans strains ATCC MYA-1003 (strain A), ATCC MYA-1237 (strain C), ATCC MYA-2310 (strain D), and ATCC 90819 (strain G) were purchased from the American Type Culture Collection (ATCC, Manassas, Va., USA). The remaining C. albicans strains, including ATCC 10231 (strain B), ATCC MYA-2876 (strain F), and ATCC 64124 (strain F) were a generous gift from Dr. Jon Y. Takemoto (Utah State University, Logan, Utah, USA). The non-albicans Candida fungi C. glabrata ATCC 2001 (strain II), C. krusei ATCC 6258 (strain L), and C. parapsilosis ATCC 22019 (strain M) were also purchased from ATCC. The C. glabrata clinical isolates, CG1 (strain I), CG2 (strain and CG3 (strain K) were a wonderful gift from Dr. Nathan P. Wiederhold (The University of Texas, San Antonio, Tex., USA). All Candida strains were grown at 35° C. on potato dextrose agar plates (PDA, catalog #110130, EMD Millipore, Billerica, Mass., USA). Liquid cultures of the yeast strains were grown in yeast extract peptone dextrose (YEPD) broth at 35° C.

The human embryonic kidney cell line HEK-293 and the human lung carcinoma epithelial cells A549 were purchased from ATCC. The human bronchial epithelial cells BEAS-2B were a generous gift from Prof. David K. Orren (University of Kentucky, Lexington, Ky.). Immortalized human keratinocytes HaCaT were an amiable gift from Prof. Hollie Swanson (University of Kentucky, Lexington, Ky.). All mammalian cells were cultured in Dulbecco's Modified Eagle's Medium (DMEM, catalog #11965-092, Thermo Fisher Scientific, Waltham, Mass.) supplemented with 10% fetal bovine serum (FBS; from ATCC) and 1% penicillin/streptomycin (from ATCC) at 37° C. with 5% CO₂.

Determination of minimum inhibitory concentration (MIC) values. MIC values were recorded for all antifungals and antihistamines in order to determine the concentration range of compounds to be used in the checkerboard assays. All antifungal agents were screened in the range of 0.063-32 μg/mL. The antihistamines, EBA and TERF, are less soluble in ddH₂O and therefore were tested starting at the maximal concentration where no precipitation was observed, 42.7 μg/mL for TERF (0.08-42.7 μg/mL) and 25 μg/mL for EBA (0.05-25 m/mL). Compound stocks for all antifungal agents and EBA were dissolved in DMSO at a concentration of 5 mg/mL while TERF was dissolved in DMSO at 10 mg/mL.

The MIC assays were done using the broth microdilution method as described in CLSI document M27-A3 with minor modifications. Yeast cultures were started by picking a colony from a PDA plate stored at 4° C. The colonies were grown in YEPD medium overnight at 35° C. with shaking at 200 rpm. In a 96-well plate compounds were diluted lengthwise along the plate in serial 2-fold dilutions in the RPMI 1640 medium. The yeast cultures were diluted in RPMI 1640 medium to an OD₆₀₀ within the range of 0.12 and 0.15 (˜1×10⁶ cells/mL). The fungal cells were further diluted by taking 25 μL of fungal cells and adding them to 10 mL of RPMI 1640 medium before adding 100 μL to plate (200 μL total volume). The plates were incubated at 35° C. for 48 h. Visual inspection of the wells for no growth was used to determine the MIC. All compounds were tested in duplicate (TABLES 6 and 7).

Combination Studies of Azoles and Antihistamines by Checkerboard Assays.

Checkerboard assays were done as previously described to determine the fractional inhibitory concentration index (FICI). 2-fold dilutions of the azole drug were made in RPMI 1640 medium lengthwise along the plate. For the antihistamine, 2-fold serial dilutions were made in sterile tubes with RPMI 1640 medium, then aliquoted into the plate. The drug concentration used in the checkerboards was 2- or 4-fold higher concentrations than the measured individual MIC values. The maximum drug concentration tested for the azoles was 32 μg/mL, while 25 μg/mL was maximal for EBA, and 42.7 m/mL for TERF. The maximal amount was used in the checkerboard assays if complete inhibition was not observed in the MIC assays. Cultures were started by picking a colony from a plate and growing in YEPD medium overnight at 35° C. with shaking at 200 rpm. Cultures were diluted in RPMI 1640 medium to an OD₆₀₀ between 0.12 and 0.15 (˜1×10⁶ cells/mL). Cells were then further diluted by taking 25 μL of cell suspension and adding to 10 mL of RPMI 1640 medium. After the drugs were added to the plate with 100 μL of medium, 100 μL of fungal cells were added. Plates were incubated at 35° C. for 48 h and visual inspection was used to determine wells with no growth. All experiments were carried out in duplicate. The FICI was calculated based on the formula below. The combinational effect of the 2 tested compounds were considered synergistic (SYN) if FICI≤0.5, additive (ADD) if 0.5<FICI≤4, and antagonistic if FICI>4 (TABLES 6-9).

$$F I C I = \frac{MIC of {azole}_{combo}}{MIC of {azole}_{alone}} + \frac{MIC of {antihistamine}_{combo}}{MIC of {antihistamine}_{alone}}$$

Time-Kill Assays.

Time-kill assays were used to assess the inhibitory efficiency of the POS and EBA combination against 2 yeast strains, C. albicans ATCC 10231 (strain B) and C. glabrata ATCC 2001 (strain H). The protocol for time-kill assays followed methods previously described with minor modifications. Yeast cultures were grown overnight in YEPD medium at 35° C. with shaking at 200 rpm. A working stock of fungal cells was made by diluting cultures in 1640 medium to an OD₆₀₀ of 0.125 (˜1×10⁶ CFU/mL). From the working stock, 100 μL of cells was added to 4.9 mL of RPMI 1640 medium in sterile culture tubes, making the starting fungal cell concentration ˜1×10⁵ CFU/mL. Drug was then added to the fungal cells. The treatment conditions included sterile control, growth control, EBA, POS, EBA and POS combination at 1×MIC, EBA and POS combination at 4×MIC, as well as AMB and NYT at their respective 1×MIC as fungicidal controls. The concentration of EBA and POS alone were the same concentration used in the combination at 1×MIC treatment. Treated fungal cultures were incubated in the culture tubes at 35° C. with 200 rpm shaking for 24 h. Samples were aliquoted from the different treatments at regular time points (0, 3, 6, 9, 12, and 24 h) and plated in duplicate. For each time point, cultures were vortexed, 100 μL of culture was aspirated, and 10-fold serial dilutions were made in sterile ddH₂O. From the appropriate dilutions, 100 μL of fungal suspension was spread on PDA plates and incubated at 35° C. for 48 h before colony counts were determined. At 24 h, 50 μL of 1 mM resazurin in phosphate buffered saline (PBS) was added to the treatments and incubated at 35° C. with 200 rpm shaking for 2 h in the dark for visual inspection. As resazurin (blue-purple) is metabolized by the cells to produce resorufin (pink-orange), the addition of resazurin is used to confirm the relative growth of the fungal cells in the different treatment conditions (FIGS. 90A-B). Experiments were performed in duplicate.

Biofilm disruption assays. Biofilm disruption assays were performed to assess the effectiveness of the POS and EBA combination against sessile yeast cells for 3 representative yeast strains, C. albicans ATCC 10231 (strain B), C. albicans ATCC 64124 (strain F), and C. glabrata ATCC 2001 (stain H). Biofilm assays were performed in 96-well plates using XTT [2,3-bis(2-methoxy-4-nitro-5-sulfophenyl)-2H-tetrazolium-5-carboxanilide] to measure the viability of the biofilm as previously described. An overnight culture of yeast was grown at 35° C. in YEPD medium with shaking at 200 rpm. The overnight culture was diluted in RPMI 1640 medium to an OD₆₀₀ between 0.12 and 0.15 to make a working stock. Working stock was transferred to the 96-well plate in 100 μL aliquots, leaving one column empty for the sterile control. The plates were incubated at 37° C. for 24 h to allow formation of the biofilm. The medium and planktonic cells from the plate were then aspirated. PBS was then used to wash any remaining planktonic cells off of the biofilm wells. 100 μL of PBS was aliquoted into wells and then removed. The wells were washed 3 times with PBS. After washing, RPMI 1640 medium and drug were added to the plate, in a similar fashion to that described in the MIC and checkerboard assays. POS was tested in the concentration range of 0.06-32 μg/mL with EBA at concentrations of 0.39-25 μg/mL in checkerboard format. As controls, the AMB and NYT SMIC were also tested in the range of 0.06-32 μg/mL (TABLE 11). Plates were incubated at 37° C. for 24 h. Finally, the plates were washed 3 times with PBS before adding 100 μL of XTT dye. The XTT was prepared by dissolving XTT at 0.5 mg/mL concentration in sterile PBS. Before adding to a plate, 1 μL of 10 mM menadione in acetone was added to 10 mL of the 0.5 mg/mL solution of XTT. After addition of XTT (containing menadione), the plates were incubated at 3 h at 37° C. in the dark. 80 μL of liquid from each well was transferred to a new plate. Plates were then read with a SpectraMax M5 plate reader (Molecular Devices, Sunnyvale, Calif., USA) for absorbance at 450 nm. For these experiments, we determined the sessile MIC (SMIC₉₀), which is defined as the drug concentration required to inhibit the metabolic activity of biofilm by 90% compared to the growth control. The SMIC₉₀ values were used to calculate the FICI as described above (TABLE 10). The plates used to determine the SMIC₉₀ are provided in FIGS. 91A-H. Each assay was performed in duplicate.

Mammalian Cytotoxicity Assays.

To examine whether the inhibitory effect of the antifungal and antihistamine combinations is specific to fungal cells, the combinations were tested with 4 mammalian cell lines: A459, BEAS-2B, HEK-293, and HaCaT. POS and EBA alone were tested against each cell line to measure their cytotoxic effect by using a resazurin cell viability assay as previously described with minor modifications. The assays were done in 96-well plates. A549 and BEAS-2B cells were plated at a density of 3×10³ cells/mL, HaCaT were plated at 2×10⁴ cells/mL, and HEK-293 cells were plated at 1×10⁴ cells/mL as determined by using a hemacytometer. POS was tested in concentrations ranging from 0.06 to 16 μg/mL and EBA was tested in the range of 0.10 to 25 μg/mL. In order to test the cytotoxic effect of the azole and antihistamine combinations, the highest concentration of EBA that did not show any cytotoxic effect when used alone (3.1 μg/mL of EBA for all cell lines) was used with varying concentrations of POS (FIGS. 92A-C). It is important to note that testing xenobiotics at sub-IC₅₀ concentrations can result in increase in cell growth, resulting in >100% cell survival in the treatment groups. In cases where >100% cell survival was observed, as custom in this field of research, we displayed the data as 100% cell survival. All assays were done in quadruplicate.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Iatta, et al., (2017) “The role of drug efflux pumps in Malassezia pachydermatis and Malasserzia furfur defence against azoles,” Mycoses. 60: 178-82.
2. Nyilasi, et al., (2010) “In vitro synergistic interactions of the effects of various statins and azoles against some clinically important fungi,” FEMS Microbiol Lett. 307: 175-84.
3. Borisy, et al., (2003) “Systematic discovery of multicomponent therapeutics,” PNAS. 100(13): 7977-82.
4. Butts, et al., (2013) “A Repurposoing Approach Identifies Off-Patent Drugs with Fungicidal Cryptococcal Activity, a Common Structural Chemotype, and Pharmacological Properties Relevant to the Treatment of Cryptococcosis,” Eukaryotic Cell. 12(2): 278-87.
5. Venturini, et al., (2011) “In vitro synergisms obtained by amphotericin B and voriconazole associated with non-antifungal agents against Fusarium spp,” Diagnostic Microbiology and Infectious Disease. 71: 126-30.
6. Quan, et al., (2006) “Potent In Vitro Synergism of Fluconazole and Berberine Chloride against Clinical Isolates of Candida albicans Resistant to Fluconazole,” Antimicroboal Agents and Chemotherapy. 50(3): 1096-99.
7. European Patent Application Publication No. 2767275, “Nortriptyline, haloperidol or prochlorperazine edisylate for use in the treatment of tuberculosis as well as two screening methods.”
8. H. X. Ngo, S. Garneau-Tsodikova, K. D. Green, A complex game of hide and seek: the search for new antifungals, MedChemComm, 7 (2016) 1285-1306.
9. M. C. Arendrup, Epidemiology of invasive candidiasis, Curr. Opin. Crit. Care, 16 (2010) 445-452.
10. P. Vandeputte, S. Ferrari, A. T. Coste, Antifungal resistance and new strategies to control fungal infections, Int. J. Microbiol., 2012 (2012) 713687.
11. M. A. Pfaller, D. J. Diekema, The Epidemiology of invasive candidiasis, in: R. A. Calderone, C. J. Clancy (Eds.) Candida and candidiasis, ASM Press, Washington, D C, 2012, pp. 449450.
12. D. W. Denning, A. Pleuvry, D. C. Cole, Global burden of allergic bronchopulmonary aspergillosis with asthma and its complication chronic pulmonary aspergillosis in adults, Med. Mycol., 51 (2013) 361-370.
13. P. E. Russell, A century of fungicide evolution, J. Agr. Sci., 143 (2005) 11-25.
14. K. Yoshida, V. J. Schuenemann, L. M. Cano, M. Pais, B. Mishra, R. Sharma, C. Lanz, F. N. Martin, S. Kamoun, J. Krause, M. Thines, D. Weigel, H. A. Burbano, The rise and fall of the Phytophthora infestans lineage that triggered the Irish potato famine, Elife, 2 (2013) e00731.
15. R. P. Singh, D. P. Hodson, J. Huerta-Espino, Y. Jin, S. Bhavani, P. Njau, S. Herrera-Foessel, P. K. Singh, S. Singh, V. Govindan, The emergence of Ug99 races of the stem rust fungus is a threat to world wheat production, Annu. Rev. Phytopathol., 49 (2011) 465-481.
16. P. G. Pappas, C. A. Kauffman, D. R. Andes, C. J. Clancy, K. A. Marr, L. Ostrosky-Zeichner, A. C. Reboli, M. G. Schuster, J. A. Vazquez, T. J. Walsh, T. E. Zaoutis, J. D. Sobel, Clinical practice guideline for the management of candidiasis: 2016 update by the infectious diseases society of America, Clin. Infect. Dis., 62 (2016) e1-e50.
17. R. Laniado-Laborin, M. N. Cabrales-Vargas, Amphotericin B: side effects and toxicity, Rev. Iberoam. Micol., 26 (2009) 223-227.
18. J. H. Rex, J. E. Bennett, A. M. Sugar, P. G. Pappas, C. M. van der Horst, J. E. Edwards, R. G. Washburn, W. M. Scheld, A. W. Karchmer, A. P. Dine, et al., A randomized trial comparing fluconazole with amphotericin B for the treatment of candidemia in patients without neutropenia. Candidemia Study Group and the National Institute, N. Engl. J. Med., 331 (1994) 1325-1330.
19. C. Charlier, E. Hart, A. Lefort, P. Ribaud, F. Dromer, D. W. Denning, O. Lortholary, Fluconazole for the management of invasive candidiasis: where do we stand after 15 years?, J. Antimicrob. Chemother., 57 (2006) 384-410.
20. M. A. Ghannoum, L. B. Rice, Antifungal agents: mode of action, mechanisms of resistance, and correlation of these mechanisms with bacterial resistance, Clin. Microbiol. Rev., 12 (1999) 501-517.
21. D. J. Sheehan, C. A. Hitchcock, C. M. Sibley, Current and emerging azole antifungal agents, Clin. Microbiol. Rev., 12 (1999) 40-79.
22. V. J. Lempers, L. C. Martial, M. F. Schreuder, N. M. Blijlevens, D. M. Burger, R. E. Aarnoutse, R. J. Bruggemann, Drug-interactions of azole antifungals with selected immunosuppressants in transplant patients: strategies for optimal management in clinical practice, Curr. Opin. Pharmacol., 24 (2015) 38-44.
23. R. Prasad, A. H. Shah, M. K. Rawal, Antifungals: Mechanism of Action and Drug Resistance, Adv. Exp. Med. Biol., 892 (2016) 327-349.
24. S. K. Shrestha, A. Garzan, S. Garneau-Tsodikova, Novel alkylated azoles as potent antifungals, Eur. J. Med. Chem., 133 (2017) 309-318.
25. V. Nagappan, S. Deresinski, Reviews of anti-infective agents: posaconazole: a broad-spectrum triazole antifungal agent, Clin. Infect. Dis., 45 (2007) 1610-1617.
26. L. Xiao, V. Madison, A. S. Chau, D. Loebenberg, R. E. Palermo, P. M. McNicholas, Three-dimensional models of wild-type and mutated forms of cytochrome P450 14alpha-sterol demethylases from Aspergillus fumigatus and Candida albicans provide insights into posaconazole binding, Antimicrob. Agents Chemother., 48 (2004) 568-574.
27. L. E. Cowen, S. D. Singh, J. R. Kohler, C. Collins, A. K. Zaas, W. A. Schell, H. Aziz, E. Mylonakis, J. R. Perfect, L. Whitesell, S. Lindquist, Harnessing Hsp90 function as a powerful, broadly effective therapeutic strategy for fungal infectious disease, Proc. Natl. Acad. Sci., U.S.A, 106 (2009) 2818-2823.
28. R. Mangoyi, J. Midiwo, S. Mukanganyama, Isolation and characterization of an antifungal compound 5-hydroxy-7,4′-dimethoxyflavone from Combretum zeyheri, BMC Complement Altern. Med., 15 (2015) 405.
29. K. Niimi, D. R. Harding, R. Parshot, A. King, D. J. Lun, A. Decottignies, M. Niimi, S. Lin, R. D. Cannon, A. Goffeau, B. C. Monk, Chemosensitization of fluconazole resistance in Saccharomyces cerevisiae and pathogenic fungi by a D-octapeptide derivative, Antimicrob. Agents Chemother., 48 (2004) 1256-1271.
30. S. K. Shrestha, M. Y. Fosso, K. D. Green, S. Garneau-Tsodikova, Amphiphilic tobramycin analogues as antibacterial and antifungal agents, Antimicrob. Agents Chemother., 59 (2015) 4861-4869.
31. S. K. Shrestha, M. Y. Fosso, S. Garneau-Tsodikova, A combination approach to treating fungal infections, Sci. Rep., 5 (2015) 17070.
32. B. Zhai, C. Wu, L. Wang, M. S. Sachs, X. Lin, The antidepressant sertraline provides a promising therapeutic option for neurotropic cryptococcal infections, Antimicrob. Agents Chemother., 56 (2012) 3758-3766.
33. J. Afeltra, P. E. Verweij, Antifungal activity of nonantifungal drugs, Eur. J. Clin. Microbiol. Infect. Dis., 22 (2003) 397-407.
34. M. Stylianou, E. Kulesskiy, J. P. Lopes, M. Granlund, K. Wennerberg, C. F. Urban, Antifungal application of nonantifungal drugs, Antimicrob. Agents Chemother., 58 (2014) 1055-1062.
35. S. Ramon-Garcia, C. Ng, H. Anderson, J. D. Chao, X. Zheng, T. Pfeifer, Y. Av-Gay, M. Roberge, C. J. Thompson, Synergistic drug combinations for tuberculosis therapy identified by a novel high-throughput screen, Antimicrob. Agents Chemother., 55 (2011) 3861-3869.
36. J. Meletiadis, J. W. Mouton, J. F. Meis, P. E. Verweij, In vitro drug interaction modeling of combinations of azoles with terbinafine against clinical Scedosporium prolificans isolates, Antimicrob. Agents Chemother., 47 (2003) 106-117.
37. R. M. Donlan, Biofilms: microbial life on surfaces, Emerg. Infect. Dis., 8 (2002) 881-890.
38. J. W. Costerton, P. S. Stewart, E. P. Greenberg, Bacterial biofilms: a common cause of persistent infections, Science, 284 (1999) 1318-1322.
39. G. Ramage, S. P. Saville, D. P. Thomas, J. L. Lopez-Ribot, Candida biofilms: an update, Eukaryot. Cell, 4 (2005) 633-638.
40. S. P. Bachmann, K. VandeWalle, G. Ramage, T. F. Patterson, B. L. Wickes, J. R. Graybill, J. L. Lopez-Ribot, In vitro activity of caspofungin against Candida albicans biofilms, Antimicrob. Agents Chemother., 46 (2002) 3591-3596.
41. M. A. Al-Fattani, L. J. Douglas, Biofilm matrix of Candida albicans and Candida tropicalis: chemical composition and role in drug resistance, J. Med. Microbiol., 55 (2006) 999-1008.
42. C. G. Pierce, P. Uppuluri, A. R. Tristan, F. L. Wormley, Jr., E. Mowat, G. Ramage, J. L. Lopez-Ribot, A simple and reproducible 96-well plate-based method for the formation of fungal biofilms and its application to antifungal susceptibility testing, Nat. Protoc., 3 (2008) 1494-1500.
43. C. L. Quave, M. Estevez-Carmona, C. M. Compadre, G. Hobby, H. Hendrickson, K. E. Beenken, M. S. Smeltzer, Ellagic acid derivatives from Rubus ulmifolius inhibit Staphylococcus aureus biofilm formation and improve response to antibiotics, PLoS One, 7 (2012) e28737.
44. B. S. Hall, C. Bot, S. R. Wilkinson, Nifurtimox activation by trypanosomal type I nitroreductases generates cytotoxic nitrile metabolites, J. Biol. Chem., 286 (2011) 1308813095.
45. W. Xu, X. Zhu, T. Tan, W. Li, A. Shan, Design of embedded-hybrid antimicrobial peptides with enhanced cell selectivity and anti-biofilm activity, PLoS One, 9 (2014) e98935.
46. M. Y. Fosso, S. K. Shrestha, K. D. Green, S. Garneau-Tsodikova, Synthesis and bioactivities of kanamycin B-derived cationic amphiphiles, J. Med. Chem., 58 (2015) 9124-9132.
47. P. Seeman, Atypical antipsychotics: mechanism of action, Can. J. Psychiatry, 47 (2002) 2940.
48. D. E. Grigoriadis, H. B. Niznik, K. R. Jarvie, P. Seeman, Glycoprotein nature of D2 dopamine receptors, FEBS Lett., 227 (1988) 220-224.
49. E. Ericson, M. Gebbia, L. E. Heisler, J. Wildenhain, M. Tyers, G. Giaever, C. Nislow, Off-target effects of psychoactive drugs revealed by genome-wide assays in yeast, PLoS Genet., 4 (2008) e1000151.
50. T. Miwa, Y. Takagi, M. Shinozaki, C. W. Yun, W. A. Schell, J. R. Perfect, H. Kumagai, H. Tamaki, Gprl, a putative G-protein-coupled receptor, regulates morphogenesis and hypha formation in the pathogenic fungus Candida albicans, Eukaryot. Cell, 3 (2004) 919-931.
51. K. Iwaki, T. Sakaeda, M. Kakumoto, T. Nakamura, C. Komoto, N. Okamura, K. Nishiguchi, T. Shiraki, M. Horinouchi, K. Okumura, Haloperidol is an inhibitor but not substrate for MDR1/P-glycoprotein, J. Pharm. Pharmacol., 58 (2006) 1617-1622.
52. R. Iatta, M. R. Puttilli, D. Immediato, D. Otranto, C. Cafarchia, The role of drug efflux pumps in Malassezia pachydermatis and Malassezia furfur defence against azoles, Mycoses, 60 (2017) 178-182
53. Hulshof, J. W.; Casarosa, P.; Menge, W. M.; Kuusisto, L. M.; van der Goot, H.; Smit, M. J.; de Esch, I. J.; Leurs, R. J. Med. Chem. 2005, 48, 6461. Suehiro, M.; Yokoi, F.; Nozaki, T.; Iwamoto, M. J. Labelled Comp. Radiopharm. 1987, 24, 1143.
54. Clinical and Laboratory Standards Institute. Reference method for broth dilution antifungal susceptibility testing of filamentous fungi-2^{nd} Edition: CLSI document M38-A2. Wayne, Pa., 2008.
55. Clinical and Laboratory Standards Institute. Reference method for broth dilution antifungal susceptibility testing of yeasts-Approved standard. CLSI document M27 A3. Wayne, Pa., 2008.
56. Garcia, L. S. In Clinical microbiology procedures handbook; 3^{rd} edition ed.; ASM Press: Washington, D C, 2010, p 140.
57. Meletiadis, J.; Mouton, J. W.; Meis, J. F.; Verweij, P. E. Antimicrob. Agents Chemother. 2003, 47, 106.
58. Klepser, M. E.; Malone, D.; Lewis, R. E.; Ernst, E. J.; Pfaller, M. A. Antimicrob. Agents Chemother. 2000, 44, 1917.
59. Pierce, C. G.; Uppuluri, P.; Tristan, A. R.; Wormley, F. L., Jr.; Mowat, E.; Ramage, G.; Lopez-Ribot, J. L. Nat. Protoc. 2008, 3, 1494.
60. Garzan, A.; Willby, M. J.; Ngo, H. X.; Gajadeera, C. S.; Green, K. D.; Holbrook, S. Y.; Hou, C.; Posey, J. E.; Tsodikov, O. V.; Garneau-Tsodikova, S. ACS Infect. Dis. 2017, 3, 302.
61. Quave, C. L.; Estevez-Carmona, M.; Compadre, C. M.; Hobby, G.; Hendrickson, H.; Beenken, K. E.; Smeltzer, M. S. PLoS One 2012, 7, e28737.
62. Hall, B. S.; Bot, C.; Wilkinson, S. R. J. Biol. Chem. 2011, 286, 13088.
63. Xu, W.; Zhu, X.; Tan, T.; Li, W.; Shan, A. PLoS One 2014, 9, e98935.
64. Shrestha, S. K.; Fosso, M. Y.; Green, K. D.; Garneau-Tsodikova, S. Antimicrob. Agents Chemother. 2015, 59, 4861.
65. Fosso, M. Y.; Shrestha, S. K.; Green, K. D.; Garneau-Tsodikova, S. J. Med. Chem. 2015, 58, 9124.
66. Hulshof, J. W., Casarosa, P., Menge, W. M., Kuusisto, L. M., van der Goot, H., Smit, M. J., de Esch, I. J., and Leurs, R. (2005) Synthesis and structure-activity relationship of the first nonpeptidergic inverse agonists for the human cytomegalovirus encoded chemokine receptor US28, J. Med. Chem. 48, 6461-6471.
67. Suehiro, M., Yokoi, F., Nozaki, T., and Iwamoto, M. (1987) No-carrier-added radiobromination via the gattermann reaction. Synthesis of 75Br- and 77Br-bromperidol, J. Labelled Comp. Radiopharm. 24, 1143-1157.
68. Mbofana, C. T., Chong, E., Lawniczak, J., and Sanford, M. S. (2016) Iron-catalyzed oxyfunctionalization of aliphatic amines at remote benzylic C-H sites, Org. Lett. 18, 4258-4261.
69. Xie, F., Bergmann, R., Kniess, T., Deuther-Conrad, W., Mamat, C., Neuber, C., Liu, B., Steinbach, J., Brust, P., Pietzsch, J., and Jia, H. (2015) (18)F-Labeled 1,4-dioxa-8-azaspiro[4.5]decane derivative: synthesis and biological evaluation of a sigmal receptor radioligand with low lipophilicity as potent tumor imaging agent, J. Med. Chem. 58, 5395-5407.
70. Subramanyam, B., Woolf, T., and Castagnoli, N., Jr. (1991) Studies on the in vitro conversion of haloperidol to a potentially neurotoxic pyridinium metabolite, Chem. Res. Toxicol. 4, 123-128,
71. Al Mubarak S, Robert A A, Baskaradoss J K, Al-Zoman K, Al Sohail A, Alsuwyed A, Ciancio S. 2013. The prevalence of oral Candida infections in periodontitis patients with type 2 diabetes mellitus. J Infect Public Health 6:296-301.
72. Atabek M E, Akyurek N, Eklioglu B S. 2013. Frequency of vaginal Candida colonization and relationship between metabolic parameters in children with type 1 diabetes mellitus. J Pediatr Adolesc Gynecol 26:257-260.
73. Nyirjesy P, Zhao Y, Ways K, Usiskin K. 2012. Evaluation of vulvovaginal symptoms and Candida colonization in women with type 2 diabetes mellitus treated with canagliflozin, a sodium glucose co-transporter 2 inhibitor. Curr Med Res Opin 28:1173-1178.
74. Spampinato C, Leonardi D. 2013. Candida infections, causes, targets, and resistance mechanisms: traditional and alternative antifungal agents. Biomed Res Int 2013:204237.
75. Austin N, Cleminson J, Darlow B A, McGuire W. 2015. Prophylactic oral/topical non-absorbed antifungal agents to prevent invasive fungal infection in very low birth weight infants. Cochrane Database Syst Rev doi:10.1002/14651858.CD003478.pub5:CD003478.
76. Hawkins D M, Smidt A C. 2014. Superficial fungal infections in children. Pediatr Clin North Am 61:443-455.
77. Horvath E E, Murray C K, Vaughan G M, Chung K K, Hospenthal D R, Wade C E, Holcomb J B, Wolf S E, Mason A D, Jr., Cancio L C. 2007. Fungal wound infection (not colonization) is independently associated with mortality in burn patients. Ann Surg 245:978-985.
78. Sarabahi S, Tiwari V K, Arora S, Capoor M R, Pandey A. 2012. Changing pattern of fungal infection in burn patients. Burns 38:520-528.
79. Wang F J, Zhang D, Liu Z H, Wu W X, Bai H H, Dong H Y. 2016. Species distribution and in vitro antifungal susceptibility of vulvovaginal Candida isolates in China. Chin Med J 129:1161-1165.
80. Goncalves B, Ferreira C, Alves C T, Henriques M, Azeredo J, Silva S. 2016. Vulvovaginal candidiasis: Epidemiology, microbiology and risk factors. Crit Rev Microbiol 42:905-927.
81. Sarbu I, Pelinescu D, Stoica I, Marutescu L, Vassu T. 2013. Phenotypic profiles of virulence in different Candida species isolated from vulvovaginal infections. Roum Arch Microbiol Immunol 72:225-233.
82. Bondaryk M, Kurzatkowski W, Staniszewska M. 2013. Antifungal agents commonly used in the superficial and mucosal candidiasis treatment: mode of action and resistance development. Postepy Dermatol Alergol 30:293-301.
83. Sobel J D, Vazquez J A. 1996. Symptomatic vulvovaginitis due to fluconazole-resistant Candida albicans in a female who was not infected with human immunodeficiency virus. Clin Infect Dis 22:726-727.
84. Perlin D S, Rautemaa-Richardson R, Alastruey-Izquierdo A. 2017. The global problem of antifungal resistance: prevalence, mechanisms, and management. Lancet Infect Dis 17:e383-e392.
85. Tobudic S, Kratzer C, Presterl E. 2012. Azole-resistant Candida spp.—emerging pathogens? Mycoses 55 (Suppl. 1):24-32.
86. Holbrook S Y L, Garzan A, Dennis E K, Shrestha S K, Garneau-Tsodikova S. 2017. Repurposing antipsychotic drugs into antifungal agents: Synergistic combinations of azoles and bromperidol derivatives in the treatment of various fungal infections. Eur J Med Chem 139:12-21.
87. Stylianou M, Kulesskiy E, Lopes J P, Granlund M, Wennerberg K, Urban C F. 2014. Antifungal application of nonantifungal drugs. Antimicrob Agents Chemother 58:1055-1062.
88. Hu Y, Sieck D E, Hsu W H. 2015. Why are second-generation H1-antihistamines minimally sedating? Eur J Pharmacol 765:100-106.
89. Hait W N, Gesmonde J F, Murren J R, Yang J M, Chen H X, Reiss M. 1993. Terfenadine (Seldane): a new drug for restoring sensitivity to multidrug resistant cancer cells. Biochem Pharmacol 45:401-406.
90. Chen C, Li G, Liao W, Wu J, Liu L, Ma D, Zhou J, Elbekai R H, Edin M L, Zeldin D C, Wang D W. 2009. Selective inhibitors of CYP2J2 related to terfenadine exhibit strong activity against human cancers in vitro and in vivo. J Pharmacol Exp Ther 329:908-918.
91. Perlmutter J I, Forbes L T, Krysan D J, Ebsworth-Mojica K, Colquhoun J M, Wang J L, Dunman P M, Flaherty D P. 2014. Repurposing the antihistamine terfenadine for antimicrobial activity against Staphylococcus aureus. J Med Chem 57:8540-8562.
92. Clinical & Laboratory Standards Institute. 2008. Reference method for broth dilution antifungal susceptibility testing of yeasts—Approved standard. CLSI document M27-A3. Wayne, Pa.
93. Garcia L S. 2016. Synergism testing: broth microdilution checkerboard and broth madrodilution methods, p 1-23, Clinical microbiology procedures handbook, 3rd edition doi:10.1128/9781555817435.ch5.12. ASM Press, Washington, D.C.
94. Meletiadis J, Mouton J W, Meis J F, Verweij P E. 2003. In vitro drug interaction modeling of combinations of azoles with terbinafine against clinical Scedosporium prolificans isolates. Antimicrob Agents Chemother 47:106-117
95. Klepser M E, Malone D, Lewis R E, Ernst E J, Pfaller M A. 2000. Evaluation of voriconazole pharmacodynamics using time-kill methodology. Antimicrob Agents Chemother 44:1917-1920.
96. Shrestha S K, Fosso M Y, Garneau-Tsodikova S. 2015. A combination approach to treating fungal infections. Sci Rep 5:17070.
97. Pierce C G, Uppuluri P, Tristan A R, Wormley F L, Jr., Mowat E, Ramage G, Lopez-Ribot J L. 2008. A simple and reproducible 96-well plate-based method for the formation of fungal biofilms and its application to antifungal susceptibility testing. Nat Protoc 3:1494-1500.
98. Garzan A, Willby M J, Ngo H X, Gajadeera C S, Green K D, Holbrook S Y, Hou C, Posey J E, Tsodikov O V, Garneau-Tsodikova S. 2017. Combating enhanced intracellular survival (Eis)-mediated kanamycin resistance of Mycobacterium tuberculosis by novel pyrrolo[1,5-a]pyrazine-based Eis inhibitors. ACS Infect Dis 3:302-309.
99. Quave C L, Estevez-Carmona M, Compadre C M, Hobby G, Hendrickson H, Beenken K E, Smeltzer M S. 2012. Ellagic acid derivatives from Rubus ulmifolius inhibit Staphylococcus aureus biofilm formation and improve response to antibiotics. PLoS One 7:e28737.
100. Hall B S, Bot C, Wilkinson S R. 2011. Nifurtimox activation by trypanosomal type I nitroreductases generates cytotoxic nitrile metabolites. J Biol Chem 286:13088-13095.
101. Xu W, Zhu X, Tan T, Li W, Shan A. 2014. Design of embedded-hybrid antimicrobial peptides with enhanced cell selectivity and anti-biofilm activity. PLoS One 9:e98935.
102. Shrestha S K, Fosso M Y, Green K D, Garneau-Tsodikova S. 2015. Amphiphilic tobramycin analogues as antibacterial and antifungal agents. Antimicrob Agents Chemother 59:4861-4869.
103. Fosso M Y, Shrestha S K, Green K D, Garneau-Tsodikova S. 2015. Synthesis and bioactivities of kanamycin B-derived cationic amphiphiles. J Med Chem 58:9124-9132.
104. Lewis R E. 2011. Current concepts in antifungal pharmacology. Mayo Clin Proc 86:805-817.
105. Sobel J D. 2016. Recurrent vulvovaginal candidiasis. Am J Obstet Gynecol 214:15-21.
106. Fleischer A B, Jr., Feldman S R. 1999. Prescription of high-potency corticosteroid agents and clotrimazole-betamethasone dipropionate by pediatricians. Clin Ther 21:1725-1731.
107. Crowley P D, Gallagher H C. 2014. Clotrimazole as a pharmaceutical: past, present and future. J Appl Microbiol 117:611-617.
108. Greenberg H L, Shwayder T A, Bieszk N, Fivenson D P. 2002. Clotrimazole/betamethasone diproprionate: a review of costs and complications in the treatment of common cutaneous fungal infections. Pediatr Dermatol 19:78-81.
109. Tang M M, Corti M A, Stirnimann R, Pelivani N, Yawalkar N, Borradori L, Simon D. 2013. Severe cutaneous allergic reactions following topical antifungal therapy. Contact Dermatitis 68:56-57.
110. Zahran K M, Agban M N, Ahmed S H, Hassan E A, Sabet M A. 2015. Patterns of Candida biofilm on intrauterine devices. J Med Microbiol 64:375-381.
111. Nobile C J, Johnson A D. 2015. Candida albicans biofilms and human disease. Ann Rev Microbiol 69:71-92.
112. Ramage G, Saville S P, Thomas D P, Lopez-Ribot J L. 2005. Candida biofilms: an update. Eukaryot Cell 4:633-638.
113. Desai J V, Mitchell A P, Andes D R. 2014. Fungal biofilms, drug resistance, and recurrent infection. Cold Spring Harb Perspect Med 4:a019729.
114. Hawser S P, Douglas L J. 1994. Biofilm formation by Candida species on the surface of catheter materials in vitro. Infect Immun 62:915-921.
115. Fonseca E, Silva S, Rodrigues C F, Alves C T, Azeredo J, Henriques M. 2014. Effects of fluconazole on Candida glabrata biofilms and its relationship with ABC transporter gene expression. Biofouling 30:447-457.
116. Llopis-Torregrosa V, Husekova B, Sychrova H. 2016. Potassium uptake mediated by Trk1 is Crucial for Candida glabrata growth and fitness. PLoS One 11:e0153374.
117. Liu J D, Wang Y J, Chen C H, Yu C F, Chen L C, Lin J K, Liang Y C, Lin S Y, Ho Y S. 2003. Molecular mechanisms of G0/G1 cell-cycle arrest and apoptosis induced by terfenadine in human cancer cells. Mol Carcinog 37:39-50.
118. Nicolau-Galmes F, Asumendi A, Alonso-Tejerina E, Perez-Yarza G, Jangi S M, Gardeazabal J, Arroyo-Berdugo Y, Careaga J M, Diaz-Ramon J L, Apraiz A, Boyano M D. 2011. Terfenadine induces apoptosis and autophagy in melanoma cells through ROS-dependent and -independent mechanisms. Apoptosis 16:1253-1267.
119. Wang W T, Chen Y H, Hsu J L, Leu W J, Yu C C, Chan S H, Ho Y F, Hsu L C, Guh J H. 2014. Terfenadine induces anti-proliferative and apoptotic activities in human hormone-refractory prostate cancer through histamine receptor-independent Mcl-1 cleavage and Bak up-regulation. Naunyn Schmiedebergs Arch Pharmacol 387:33-45.
120. Segura-Cabrera A, Tripathi R, Zhang X, Gui L, Chou T F, Komurov K. 2017. A structure- and chemical genomics-based approach for repositioning of drugs against VCP/p97 ATPase. Sci Rep 7:44912.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound selected from the group consisting of:

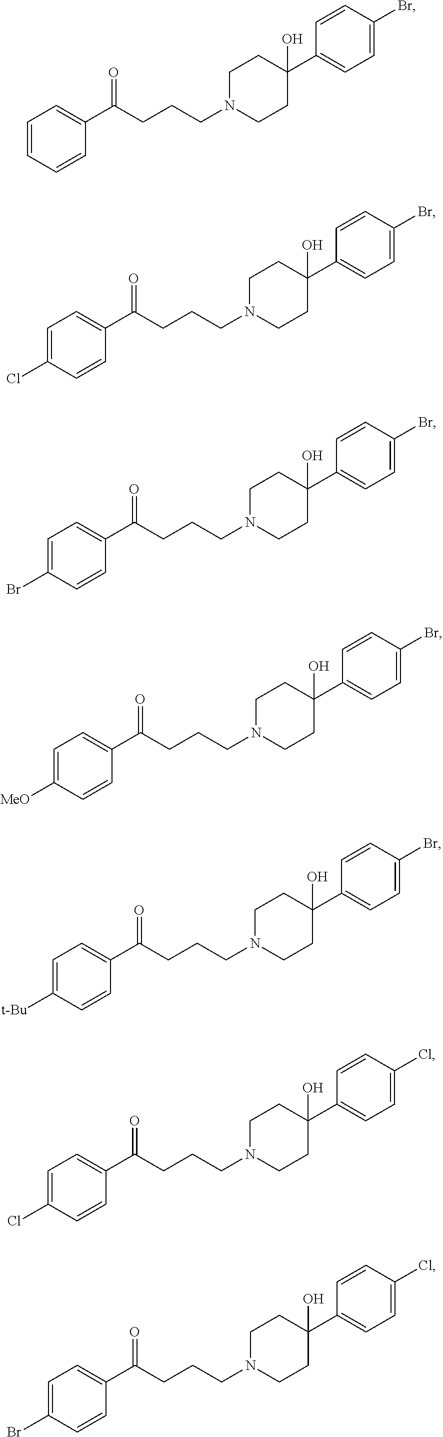

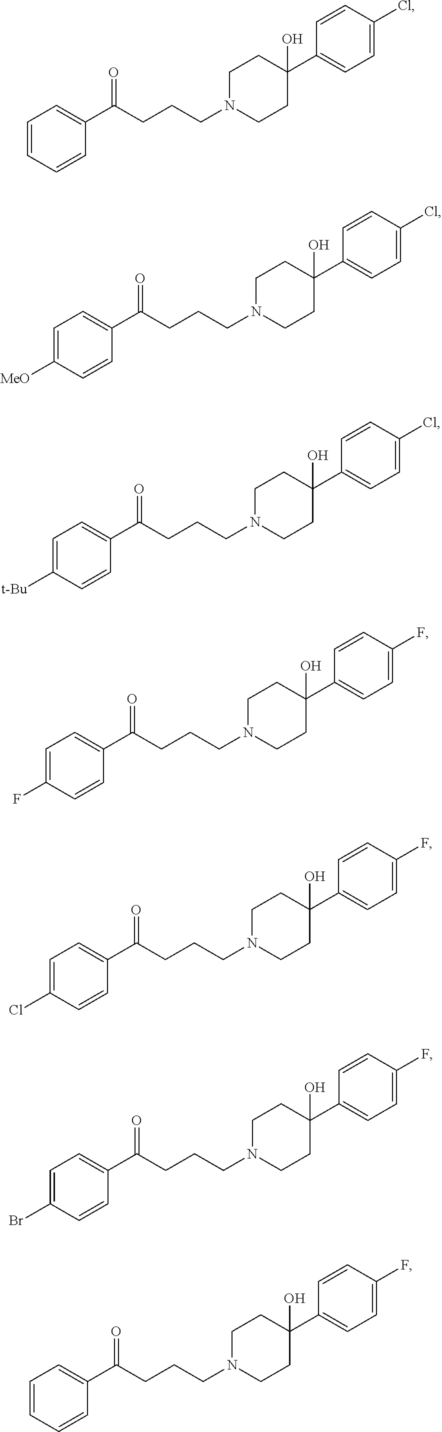

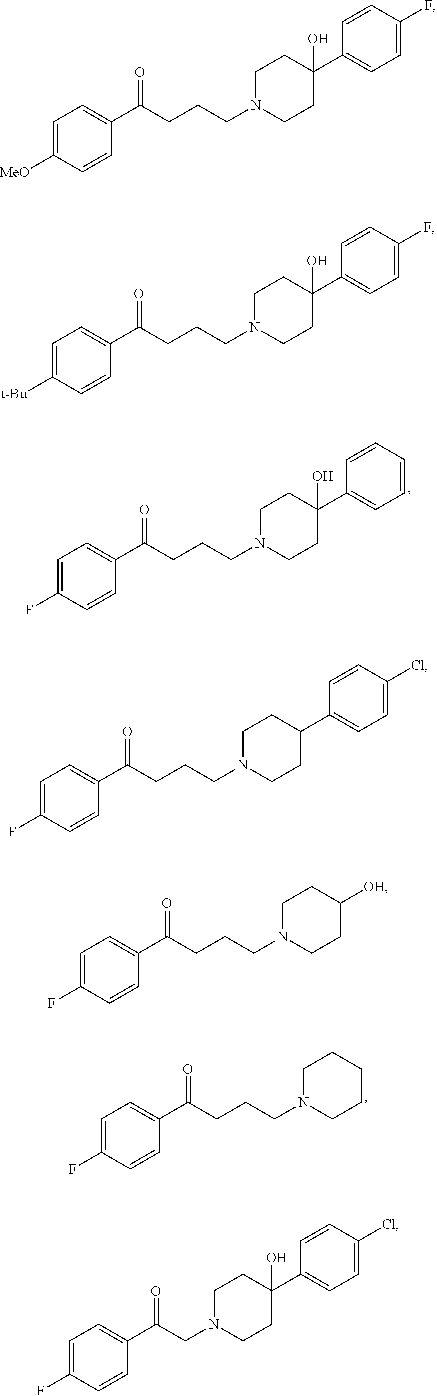

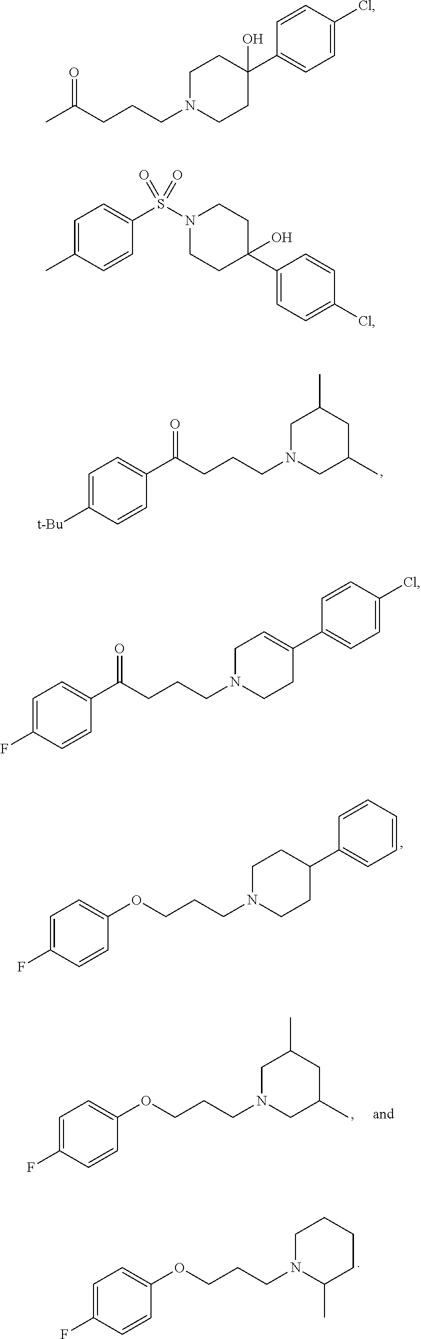

2. A composition, comprising:
an antifungal agent; and
a compound selected from the group consisting of: 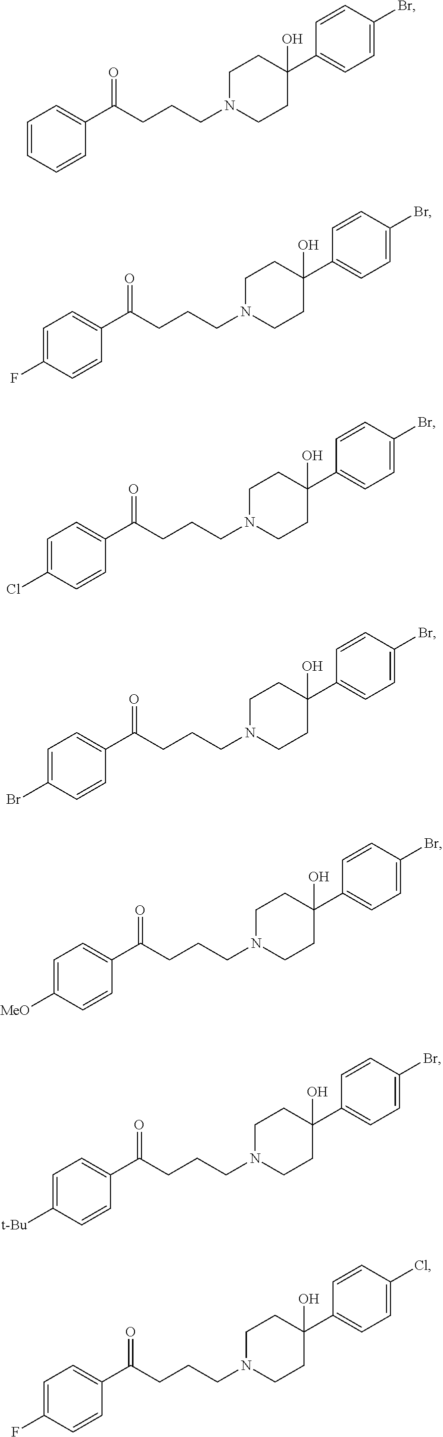 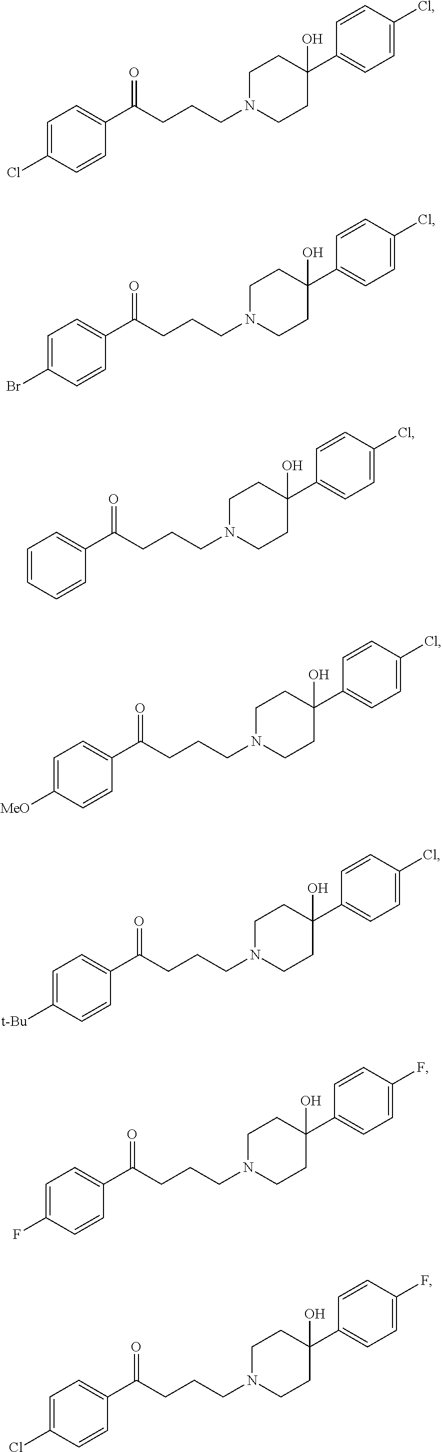 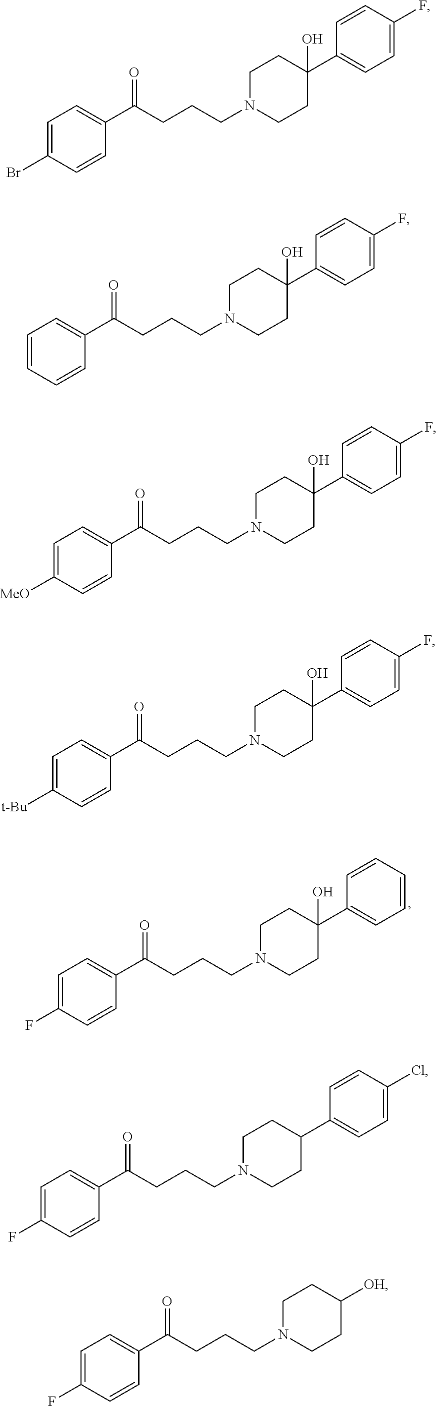 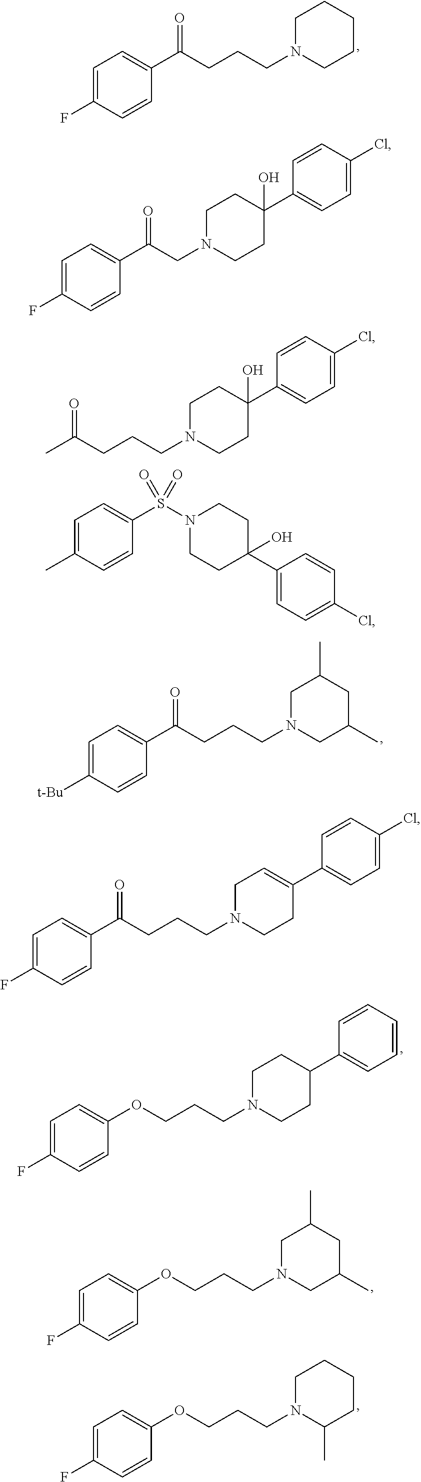
terfenadine (TERF), ebastine (EBA), and derivatives thereof.

3. The composition of claim 2, wherein the antifungal agent is an azole antifungal agent.

4. The composition of claim 2, wherein the antifungal agent is a non-azole antifungal agent.

5. The composition of claim 2, wherein the antifungal agent is selected from the group consisting of: fluconazole (FLC), itraconazole (ITC), ketoconazole (KTC), posaconazole (POS), and voriconazole (VOR), ketoconazolev(KTC), undecylic acid (undecanoic acid), nystatin (NYS), naftifine (NAF), tolnaftate, amorolfine, butenafine (BTF), miconazole (MCZ), econazole, ciclopirox, oxiconazole, sertaconazole, efinaconazole, clotrimazole (CLO), sulconazole, tioconazole, tavaborole, terbinafine (TER), mancozeb, tricyclazole, carbendazim, hexaconazole, propineb, metalaxyl, benomyl (BEN) (Methyl 1-(butylcarbamoyl)-2-benzimidazolecarbamate), difenoconazole, propiconazole (PCZ), kitazin, tebuconazole (TER), tridemorph (TDM), and metconazole (MET).

6. The composition of claim 2, wherein the composition has increased antifungal activity as compared to the antifungal agent alone.

7. The composition of claim 2, wherein the composition has synergistically increased antifungal activity as compared to the antifungal agent alone.

8. The composition of claim 2, wherein the ratio of antifungal agent to compound is between about 1:1 and 1:1100.

9. The composition of claim 2, wherein the ratio of antifungal agent to compound is between about 1:1 and 1:5.

10. The composition of claim 2, wherein the ratio of antifungal agent to compound is between about 1:1 and 1:10.

11. The composition of claim 2, wherein the ratio of antifungal agent to compound is between about 1:1 and 1:100.

12. The composition of claim 2, wherein the ratio of antifungal agent to compound is between about 1:1 and 1:300.

13. The composition of claim 2, wherein the ratio of antifungal agent to compound is between about 1:1 and 1:500.

14. The composition of claim 2, wherein the ratio of antifungal agent to compound is between about 1:10 and 1:500.

15. The composition of claim 2, wherein the effective amount of the antifungal agent in the composition is less than the effective amount of the antifungal agent when used alone.

16. A method of treating a fungal infection, comprising administering the composition of claim 2 to an infected plant.

17. The method of claim 16, wherein the infection is caused by a plant pathogen.

18. A method of treating a fungal infection, comprising administering the composition of claim 2 to a subject in need thereof.

19. The method of claim 18, wherein the infection is caused by an animal pathogen.

20. The method of claim 18, wherein the infection is caused by a human pathogen.
